(19) United States
(12) Patent Application Publication
(10) Pub. No.: US 2020/0131586 A1
(43) Pub. Date: Apr. 30, 2020

(54) METHODS AND COMPOSITIONS FOR DIAGNOSING OR DETECTING LUNG CANCERS

(71) Applicant: The Wistar Institute of Anatomy and Biology, Philadelphia, PA (US)

(72) Inventors: Louise C. Showe, Media, PA (US); Michael K. Showe, Media, PA (US); Andrei V. Kossenkov, Huntingdon Valley, PA (US)

(21) Appl. No.: 16/725,767

(22) Filed: Dec. 23, 2019

Related U.S. Application Data
(63) Continuation of application No. 15/574,737, filed on Nov. 16, 2017, now abandoned.
(60) Provisional application No. 62/163,766, filed on May 19, 2015.

(51) Int. Cl. C12Q 1/6886; G16H 50/70; G16B 40/00; G16B 25/00

(52) U.S. Cl. CPC C12Q 1/6886; G16H 50/70; G16B 40/00; G16B 25/00; C12Q 2600/158; C12Q 2600/112; C12Q 2600/106; C12Q 2600/178; C12Q 2600/118

(57) ABSTRACT

A multi-analyte composition for the diagnosis of lung cancer or lung disease comprises a ligand selected from a nucleic acid sequence, polynucleotide or oligonucleotide capable of specifically complexing with, hybridizing to, or identifying an mRNA gene transcript from a mammalian blood sample, and an additional ligand selected from a nucleic acid sequence, polynucleotide or oligonucleotide capable of specifically complexing with, hybridizing to, or identifying an miRNA of a gene from a mammalian blood sample. Each ligand and additional ligand binds to a different gene transcript or miRNA and the gene transcripts and miRNA identified form a characteristic profile of a stage of lung cancer or lung disease. Methods of using this composition for diagnosis and evaluation and methods for developing such compositions are described.

### CROSS-REFERENCE TO RELATED APPLICATIONS

This application is a continuation of U.S. patent application Ser. No. 15/574,737, filed Nov. 16, 2017, which is a national stage of International Patent Application No. PCT/US2016/033232, filed May 16, 2016, which claims the benefit of the priority of U.S. Provisional Patent Application No. 62/163,766, filed May 19, 2015 (expired), which applications are incorporated herein by reference.

### STATEMENT OF GOVERNMENT INTEREST

This invention was made with government support under Grant Nos. P30 CA010815 awarded by the National Institutes of Health. The government has certain rights in the invention.

### INCORPORATION BY REFERENCE OF MATERIAL SUBMITTED IN ELECTRONIC FORM

Applicant hereby incorporates by reference the Sequence Listing material filed in electronic form herewith. This file is labeled “WST155PCT_ST25.txt”, was created on May 19, 2016, and is 43 KB.

### BACKGROUND OF THE INVENTION

Lung cancer is the most common worldwide cause of cancer mortality, accounting for about 220,000 newly diagnosed cases each year or about 13% of all cancer diagnoses. Over 27% of all cancer deaths are due to lung cancer, about 150,000 deaths each year. Current rates of diagnosis are late stage, i.e., greater than >70% of diagnoses are stage III and above and only 15% of such lung cancers are diagnosed at an earlier, treatable stage, i.e., Stage I or IIA. Survival rates for lung cancer overall are about 18% five-year survival, contrasted with. >50% 5 year survival rates for diagnosis at an early stage of the disease.

Non-small cell lung cancer (NSCLC) is a highly lethal disease with cure only possible by early detection followed by surgery. Unfortunately, at the time of diagnosis only 15% of patients with lung cancer have localized disease. Field cancerization in which the lung epithelium becomes mutagenized following exposure to cigarette smoke makes it difficult to identify genetic changes that differentiate smokers from smokers with early lung cancer. One of the most important long-term goals in improving lung cancer survival is to achieve detection of malignant tumors in patients, primarily smokers and former smokers, who represent the majority of all lung cancer cases, at an early stage, while they are still surgically resectable. Currently, the only way to differentiate benign from malignant nodules is an invasive biopsy, surgery, or prolonged observation with repeated scanning. Approaches to early diagnosis involve processes, such as CT scan, bronchial brushing, and the analysis of sputum, plasma, and blood for biomarkers of disease.

One established and validated method to achieve the goal of genetic diagnosis has been the use of microarray signatures from tumor tissue. Peripheral blood mononuclear cells (PBMC) profiles can be used to diagnose and classify systemic diseases, including cancer, and to monitor therapeutic response. The validity of using PBMC gene expression profiles in patients with cancer has been previously reported in the use of microarrays to compare PBMC from patients with late stage renal cell carcinoma compared to normal controls. A 37 gene classifier has been developed for detecting early breast cancer from peripheral blood samples with 82% accuracy. Another study identified gene expression profiles in the PBMC of colorectal cancer patients that could be correlated with response to therapy. The inventors also determined a 29 gene classifier for disease in patient PBMC (see, e.g., U.S. Pat. No. 8,476,420, incorporated by reference herein).

MicroRNAs (miRNAs) are a large group of non-coding ribonucleic acid sequences, isolated and identified from insects, microorganisms, humans, animals and plants, which are reported in databases including that of The Wellcome Trust Sanger Institute (http://miRNA.sanger.ac.uk/sequences/). These miRNAs are about 22 nucleotides in length and arise from longer precursors, which are transcribed from non-protein-encoding genes. The precursors form structures that fold back on themselves in self-complementary regions. Relatively little is known about the functional role of miRNAs and even less on their targets. It is believed that miRNA molecules interrupt or suppress gene translation through precise or imprecise base-pairing with their targets (US Published Patent Application No. 2004/0175732). Bioinformatics analyses suggest that any given miRNA may bind to and alter the expression of up to several hundred different genes; and a single gene may be regulated by several miRNAs. The complicated interactive regulatory networks among miRNAs and target genes have been noted to make it difficult to accurately predict which genes will actually be improperly regulated in response to a given miRNA. Expression levels of certain miRNAs have been associated with various cancers (Esquela-Kerscher and Slack, 2006 Nat. Rev. Cancer, 6(4):259-269; McManus 2003 Seminars in Cancer Biology, 13:253-258; Karube Y et al 2005 Cancer Sci, 96(2):111-5; Yanaihara N. et al 2006 Cancer Cell, 9(3):189-98).

The inventors have previously disclosed in International Patent Application Publication No. WO2010/054233, filed Nov. 6, 2009, a diagnostic reagent or kit comprising a ligand capable of specifically complexing with, hybridizing to, or identifying miRNAs and particularly an miRNA profile that includes various combinations of hsa-miR-148a, hsa-miR-142-5p, hsa-miR-221, hsa-miR-let-7d, hsa-miR-let-7a, hsa-miR-328, hsa-miR-let-7c, hsa-miR-34a, hsa-miR-202, hsa-miR-769-5p, hsa-miR-642. These reagents and kits are useful in methods of diagnosing or detecting lung cancer in a mammalian subject by identifying the miRNA expression levels or profiles of these miRNA in a subject's whole blood or peripheral blood mononuclear cells.

There remains a need in the art for new and effective tools to facilitate early diagnoses of various lung cancers and other lung diseases.

### SUMMARY OF THE INVENTION

In one aspect, a multi-analyte composition is provided for the diagnosis or evaluation of a mammalian subject suspected of having lung cancer or a lung disease. This composition is a reagent or kit and involves ligands that permit the identification of changes in the expression of certain mRNA (gene transcripts) and non-coding miRNA in a mammalian biological sample. The combined changes in these selected coding and non-coding sequences permit the identification of a profile or classification of sequences that change in response to the presence, stage or progression of a lung cancer or lung disease.

In one embodiment, the ligands are probes that bind to certain mRNA and miRNA provided in Table 1 below.

In another aspect, methods are provided for using a multi-analyte composition to diagnose the presence, stage or progression of a lung cancer or lung disease.

In yet a further aspect, methods for developing characteristic lung cancer classifications or combined mRNA and miRNA profiles that enable diagnosis of lung cancer, lung disease, or a stage or subtype thereof are provided.

In another aspect, a method for increasing the sensitivity and specificity of an assay for discriminating between subjects with lung cancer and subjects with benign nodules is provided.

In another aspect, a multi-analyte composition is provided for the diagnosis or evaluation of a mammalian subject suspected of having lung cancer or a lung disease, which is a reagent or kit and involves ligands that permit the identification of changes in the expression of certain mRNA targets (gene transcripts) in a mammalian biological sample. The mRNA targets are multiple targets selected from Tables 1, 2 and 3 herein.

Other aspects and advantages of these compositions and methods are described further in the following detailed description of the preferred embodiments thereof.

### BRIEF DESCRIPTION OF THE DRAWINGS

FIG. 1 is a graph showing the estimation of error rate for training sets of increasing size. The power function curve was fit by selecting different training sets sizes from the overall data. MAD: median absolute deviation across 50 resamplings. The Power curve was developed on our preliminary studies of samples described in methods. The power function was fit by selecting different training set sizes from the overall data and plotting it against the corresponding error rate of the classification for that data. The relationship between the numbers of samples used for training and the error rate shows that, by increasing the training set size, we can achieve higher accuracies in the classification of NSCLC versus controls with and without nodules. 90% classification accuracy can be achieved by using a training set containing approximately 550 samples. The results for the 242 samples used for the training in the examples are indicated in green on the curve; the error rate of this analysis is 0.17 and is right on the target with our earlier prediction. MAD: median absolute deviation across 50 re-samplings.

FIG. 2 is a graph showing the ROC AUC for the combined classifier of Example 3. This data was obtained using 242 training samples and 103 test samples, e.g., Cancer vs. controls. Accuracies comparison showed mRNA only at 79%, miRNA only at 71%, but the combination of mRNA and miRNA at 83%. Sensitivity of the assay was 76%. Specificity of the assay was 88% and ROC AUC was 0.88. Cancer subjects (n=54); controls (n=49).

FIG. 3 is a Support Vector Machines (SVM) plot showing the individual scores for each sample from the independent testing set assigned by the classifier. Each sample received a score assigned by the SVM classifier. Positive scores indicate classification as cancer and negative scores as a control. Each column represents a patient and the height of the column can be interpreted as a measure of the strength or the reliability of the classification. The classification shown uses the classical 0 point cutoff for classification. The sensitivity maximizes at 92.6% with Specificity at 73.5%. The SVM analysis assigns a score to each sample which is a measure of how well each is classified.

FIG. 4 is a flow chart demonstrating the number and evaluation of biological samples employed in developing classifiers comprised of mRNA and miRNA targets for diagnosis of lung disease.

### DETAILED DESCRIPTION

The inventors developed an algorithm for a classification that was SVM with forward feature selection. mRNA and miRNA were analyzed separately to develop independent classifiers and to demonstrate a synergistic level of accuracy surpassing that of using just mRNA or just miRNA to make a diagnosis. A combined classifier was developed by combining coding and non-coding features, which permits a diagnosis with improved accuracy.

The combined mRNA and/or miRNA expression (combined classifier) is more accurate when compared to preliminary PBMC using miRNA results only. The multi-analyte classifier is more robust. More features are needed for classification; and these feature numbers may be reduced with larger training set, but number is compatible with potential development platforms, such as Nanostring (Nanostring Technologies, Inc., Seattle, Wash.) and PCR arrays.

The methods and compositions described herein apply combined detection of selected gene transcripts (mRNA) and detection of selected miRNA (non-coding) expression technology to screening of biological fluid for the detection, diagnosis, and monitoring of response to treatment of a condition, such as a lung disease. In certain embodiments, the lung disease is an NSCLC or COPD. In other embodiments the disease is the presence of benign nodes. Still other lung diseases are diagnosed using the compositions described herein. The compositions and methods described herein permit the diagnosis or detection of a condition or disease or its stage generally, and lung cancers and COPD particularly, by determining changes in combined characteristic gene transcripts (mRNA) and characteristic miRNA or miRNA expression profiles (non-coding) derived from a biological sample. The sample includes in various embodiments, whole blood, serum or plasma of a mammalian, preferably human, subject. The combined changes in expression of both mRNA targets and miRNA targets is established by comparing the profiles of numerous subjects of the same class (e.g., patients with a certain type and stage of lung cancer or COPD, or a mixture of types and stages) with numerous subjects of a class from which these individuals must be distinguished in order to provide a useful diagnosis.

These methods of lung disease screening employ compositions suitable for conducting a simple and cost-effective and non-invasive blood test using combined mRNA and miRNA expression profiling that could alert the patient and physician to obtain further studies, such as a chest radiograph or CT scan, in much the same way that the prostate specific antigen is used to help diagnose and follow the progress of prostate cancer. The mRNA and miRNA expression levels and profiles described herein provide the basis for a variety of classifications related to this diagnostic problem. The application of these comparative levels and profiles provides overlapping and confirmatory diagnoses of the type of lung disease, beginning with the initial test for malignant vs. non-malignant disease.

### Components of the Compositions and Methods

“Patient” or “subject” as used herein means a mammalian animal, including a human, a veterinary or farm animal, a domestic animal or pet, and animals normally used for clinical research. More specifically, the subject of these methods and compositions is a human.

“Ligand”, as used herein, refers to any nucleotide sequence, amino acid sequence, antibody, probes, primers, fragments thereof or any entity (small molecule or chemical or recombinant molecules), labeled or unlabeled, that is able to hybridize to, bind to, or otherwise associate with the target mRNA or miRNA, so as to permit detection and quantitation of the target mRNA or miRNA.

“Reference” level, standard or profile as used herein refers to the source of the reference mRNA and miRNA. In one embodiment, the reference mRNA and miRNA standards are obtained from biological samples selected from a reference human subject or population having a non-small cell lung cancer (NSCLC). For example, in one embodiment, the reference standard utilized is a standard or profile derived from biological samples of a reference human subject or population of human subjects with squamous cell carcinoma or an average of multiple subjects with squamous cell carcinoma. In certain embodiments, the reference standard utilized is a standard or profile derived from a reference human subject, or an average of multiple subjects, with early stage squamous cell carcinoma. In another embodiment, the reference standard is a standard or profile derived from a reference human subject, or an average of multiple subjects, with adenocarcinoma. In another embodiment, the reference standard is a standard or profile derived from the biological samples of a reference human subject, or an average of multiple subjects, with early stage adenocarcinoma.

In another embodiment, the reference mRNA and miRNA standards are obtained from biological samples selected from a reference human subject or population having COPD or some other pulmonary disease. For example, the reference standard is a standard or profile derived from the biological sample of a reference human subject, or an average of multiple subjects, with COPD. In one embodiment, the reference mRNA and miRNA standard is obtained from biological samples selected from a reference human subject or population who are healthy and have never smoked. For example, the reference standard is a standard or profile derived from the biological sample of a reference human subject, or an average of multiple subjects, who are healthy and have never smoked. In one embodiment, the reference mRNA and miRNA standards are obtained from biological samples selected from a reference human subject or population who are former smokers or current smokers with no disease. For example, the reference standard is a standard or profile derived from a reference human subject, or an average of multiple subjects, who are former smokers or current smokers with no disease.

In one embodiment, the reference mRNA and miRNA standard is obtained from biological samples selected from a reference human subject or population having benign lung nodules. For example, the reference standard is a standard or profile derived from the biological sample of a reference human subject, or an average of multiple subjects, who have benign lung nodules. In one embodiment, the reference mRNA and miRNA standard is obtained from biological samples selected from a reference human subject or population following surgical removal of an NSCLC tumor. In one embodiment, the reference mRNA and miRNA standard is obtained from biological samples selected from a reference human subjects or population prior to surgical removal of an NSCLC tumor. In one embodiment, the reference mRNA and miRNA standard is obtained from biological samples selected from the same subject who provided a temporally earlier biological sample. In another embodiment, the reference standard is a combination of two or more of the above reference standards.

The reference standard, in various embodiments, is a mean, an average, a numerical mean or range of numerical means, a numerical pattern, a graphical pattern or an miRNA or mRNA or gene expression profile derived from a reference subject or reference population. Selection of the particular class of reference standards, reference population, mRNA levels or profiles or miRNA levels or profiles depends upon the use to which the diagnostic/monitoring methods and compositions are to be put by the physician.

“Sample” or “Biological Sample” as used herein means any biological fluid or tissue that contains immune cells and/or cancer cells. In one embodiment, a suitable sample is whole blood. In another embodiment the sample may be venous blood. In another embodiment, the sample may be arterial blood. In another embodiment, a suitable sample for use in the methods described herein includes peripheral blood, more specifically peripheral blood mononuclear cells. Other useful biological samples include, without limitation, whole blood, plasma, or serum. In still other embodiment, the sample is saliva, urine, synovial fluid, bone marrow, cerebrospinal fluid, vaginal mucus, cervical mucus, nasal secretions, sputum, semen, amniotic fluid, bronchoalveolar lavage fluid, and other cellular exudates from a subject suspected of having a lung disease. Such samples may further be diluted with saline, buffer or a physiologically acceptable diluent. Alternatively, such samples are concentrated by conventional means. It should be understood that the use or reference throughout this specification to any one biological sample is exemplary only. For example, where in the specification the sample is referred to as whole blood, it is understood that other samples, e.g., serum, plasma, etc., may also be employed in the same manner.

In one embodiment, the biological sample is whole blood, and the method employs the PaxGene Blood RNA Workflow system (Qiagen). That system involves blood collection (e.g., single blood draws) and RNA stabilization, followed by transport and storage, followed by purification of Total RNA and Molecular RNA testing. This system provides immediate RNA stabilization and consistent blood draw volumes. The blood can be drawn at a physician's office or clinic, and the specimen transported and stored in the same tube. Short term RNA stability is 3 days at between 18-25° C. or 5 days at between 2-8° C. Long term RNA stability is 4 years at −20 to −70° C. This sample collection system enables the user to reliably obtain data on gene expression and miRNA expression in whole blood. In one embodiment, the biological sample is whole blood. While the PAXgene system has more noise than the use of PBMC as a biological sample source, the benefits of PAXgene sample collection outweighs the problems. Noise can be subtracted bioinformatically.

“Immune cells” as used herein means B-lymphocytes, T-lymphocytes, NK cells, macrophages, mast cells, monocytes and dendritic cells.

As used herein, the term “condition” refers to the absence (healthy condition) or presence of a disease including a lung disease, a lung cancer, the presence of benign nodules or benign tumor growths in the lung, chronic obstructive pulmonary disease (with or without associated cancer), the existence of a cancerous lung tumor prior to surgery, the post-surgical condition after removal of a cancerous lung tumor. Where specified, any of such conditions can be associated with smoking or not-smoking.

As used herein, the term “lung disease” refers to a lung cancer or chronic obstructive pulmonary disease, or the presence of lung nodules or lung lesions due to smoking or some other adverse even in the lung tissue.

As used herein the term “cancer” refers to or describes the physiological condition in mammals that is typically characterized by unregulated cell growth. More specifically, as used herein, the term “cancer” means any lung cancer. In one embodiment, the lung cancer is non-small cell lung cancer (NSCLC). In a more specific embodiment, the lung cancer type is lung adenocarcinoma (AC). In another embodiment, the lung cancer type is lung squamous cell carcinoma (SCC). In another embodiment, the lung cancer is an “early stage” (I or II) NSCLC. In still another embodiment, the lung cancer is a “late stage” (III or IV) NSCLC. In still another embodiment, the lung cancer is a mixture of early and late stages and types of NSCLC.

The term “tumor,” as used herein, refers to all neoplastic cell growth and proliferation, whether malignant or benign, and all pre-cancerous and cancerous cells and tissues.

By “diagnosis” or “evaluation” refers to a diagnosis of a lung cancer, a diagnosis of a stage of lung cancer, a diagnosis of a type or classification of a lung cancer, a diagnosis or detection of a recurrence of a lung cancer, a diagnosis or detection of a regression of a lung cancer, a prognosis of a lung cancer, an evaluation of the response of a lung cancer to a surgical or non-surgical therapy, or a diagnosis of benign lung nodules.

By “change in expression” is meant an upregulation of one or more selected gene transcripts (RNA) or miRNAs in comparison to the reference or control; a downregulation of one or more selected genes or miRNAs in comparison to the reference or control; or a combination of certain upregulated genes or miRNAs and down regulated genes or miRNAs.

By “therapeutic reagent” or “regimen” is meant any type of treatment employed in the treatment of cancers with or without solid tumors, including, without limitation, chemotherapeutic pharmaceuticals, biological response modifiers, radiation, diet, vitamin therapy, hormone therapies, gene therapy, surgical resection, etc.

By “selected or specified” mRNAs or “selected or specified” miRNAs as used herein is meant those mRNA and miRNA sequences, the combined expression of which changes (either in an up-regulated or down-regulated manner) characteristically in the presence of a condition such as a lung disease or lung cancer. In one embodiment, the selected mRNAs and miRNAs are those reported in Tables 1-3. A statistically significant number of such informative mRNAs and miRNAs form a suitable combined mRNA and miRNA expression profile for use in the methods and compositions. The statistically significant number is determined based upon the ability of same to discriminate between two or more of the tested reference populations.

The term “statistically significant number of mRNAs and miRNAs” in the context of this invention differs depending on the degree of change in combined mRNA and miRNA expression observed. The degree of change in mRNA and miRNA expression varies with the condition, such as type of lung disease or cancer and with the size or spread of the cancer or solid tumor. The degree of change also varies with the immune response of the individual and is subject to variation with each individual. The degree of change in expression of the specified mRNA and miRNAs varies with the type of disease diagnosed, e.g., COPD or NSCLC, and with the size or spread of the cancer or solid tumor. The degree of change also varies with the immune response of the individual and is subject to variation with each individual. For example, in one embodiment of this invention, a change at or greater than a 1.2 fold increase or decrease in expression of a combined mRNA miRNA or more than two such mRNA and miRNA, or even 3 to about 119 or 145 or 200 or more characteristic combined mRNA and miRNA, is statistically significant. In another embodiment, a larger change, e.g., at or greater than a 1.5 fold, greater than 1.7 fold or greater than 2.0 fold increase or decrease in expression of a combined mRNA and miRNA or more than two such mRNA or miRNA, or even 3 to about 119 or more characteristic combined mRNA and miRNA, is statistically significant. This is particularly true for cancers without solid tumors. Still alternatively, if a single combination of an mRNA and an miRNA is profiled as up-regulated or expressed significantly in cells which normally do not express the mRNA or miRNA, such up-regulation of a single mRNA and/or miRNA may alone be statistically significant. Conversely, if a single combination of mRNA and miRNA is profiled as down-regulated or not expressed significantly in cells which normally do express the combination of the mRNA and miRNA, such down-regulation of a single combined set may alone be statistically significant.

Thus, the methods and compositions described herein contemplate examination of the expression level or profile of from 1 to about 200 combined mRNA and miRNA in a single profile (see Tables 1 and 2). In another embodiment, the methods and compositions described herein contemplate examination of the expression level or profile of from 1 to about 119 (by ranking in Table 1) of the combined mRNA and miRNA in a single profile. In another embodiment, the methods and compositions described herein contemplate examination of the expression level or profile of from 1 to about 145 (by ranking in Table 1) of the combined mRNA and miRNA in a single profile. In another embodiment, the methods and compositions described herein contemplate examination of the expression level or profile of from 1 to about 147 (by ranking in Table 2) of the combined mRNA and miRNA in a single profile. In another embodiment, the methods and compositions described herein contemplate examination of the expression level or profile of from 1 to about 200 combined mRNA and miRNA in a single profile, having the mRNA and miRNA identified in Table 3. In still another embodiment, combinations of only some mRNAs from Tables 1-3 or some miRNAs from Tables 1-3 are useful as profiles for use in diagnosing patients with a lung cancer or lung.

In one embodiment, a significant change in the expression level of one of the identified combinations of mRNA and/or miRNA can be diagnostic of a condition, e.g., lung disease. In another embodiment, a significant change in the expression level of two of the identified mRNA and/or miRNAs can indicate a condition, e.g., a lung disease. In another embodiment, a significant change in the expression level of a combination of three of the identified mRNA and/or miRNAs can be diagnostic of a lung disease or indicate another condition. The combinations of mRNA and/or miRNA need not be equal in number in an expression profile. For example, as in the set of the first ranked 119 components of Table 1, the mRNAs can outnumber the miRNAs in a combination. In another embodiment, a significant change in the expression level of four or more of the identified mRNAs and/or miRNAs can be diagnostic of a lung disease or indicate another condition. In another embodiment, a significant change in the expression level of at least 10, at least 50, at least 100, at least about 119 or at least about 145 (or any integer between any of these endpoints) of the identified combination of mRNAs and miRNAs of Table 1 is diagnostic of a lung disease or indicate another condition.

In another embodiment, a significant change in the expression level of four or more of the identified mRNAs and/or miRNAs can be diagnostic of a lung disease or indicate another condition. In another embodiment, a significant change in the expression level of at least 10, at least 50, at least 100, at least 120 or at least about 147 (or any integer between any of these endpoints) of the identified combination of mRNAs and miRNAs of Table 2 is diagnostic of a lung disease or indicate another condition.

In another embodiment, a significant change in the expression level of at least 10, at least 15, at least 20 (or any integer between any of these endpoints) of the identified combination of mRNAs and miRNAs of Table 3 is diagnostic of a lung disease or indicate another condition.

In another embodiment, a significant change in the expression level of about 15 of the selected combinations of mRNA and miRNAs can be diagnostic of a lung disease or indicate another condition. In another embodiment, a significant change in the expression level of about 20 to 40 of the identified combinations of mRNAs and miRNAs can be diagnostic of a lung disease or indicate another condition. Still other numbers of mRNAs combined with miRNA changes can be used in diagnosis of lung disease or indicate another lung condition as taught herein. In still a further embodiment, a profile of mRNAs diagnostic of a lung disease or another condition includes five or more of the mRNAs ranked as 2, 5, 7, 10, 12, 15, 17, 24, 26, 27, 31, 36, 40, 41, 46, 51, 57, 58, 63, 69, 78, 80, 85, 94, 101, 105, 107, 117, 118, 125 127, 128, 134 and 139 in Table 1 below. Still other groups of the mRNAs and/or miRNAs may be selected from within Table 1, Table 2 or Table 3.

The term “microarray” refers to an ordered arrangement of hybridizable array elements. In one embodiment, a microarray comprises polynucleotide probes that hybridize to the specified combination of mRNA and miRNA, on a substrate. In another embodiment, a microarray comprises multiple primers or antibodies, optionally immobilized on a substrate.

A change in expression of an combination of a mRNA and/or miRNA required for diagnosis or detection by the methods described herein refers to an mRNA or miRNA whose expression is activated to a higher or lower level in a subject having a condition or suffering from a disease, specifically lung cancer or NSCLC, relative to its expression in a reference subject or reference standard. mRNAs and miRNAs may also be expressed to a higher or lower level at different stages of the same disease or condition. Expression of specific combinations of mRNAs and miRNAs differ between normal subjects who never smoked or are current or former smokers, and subjects suffering from a disease, specifically COPD, benign lung nodules, or cancer, or between various stages of the same disease. Expression of specific mRNAs and miRNAs differ between pre-surgery and post-surgery patients with lung cancer. Such differences in miRNA expression include both quantitative, as well as qualitative, differences in the temporal or cellular expression patterns among, for example, normal and diseased cells, or among cells which have undergone different disease events or disease stages. For the purpose of this invention, a significant change in combined mRNA and miRNA expression when compared to a reference standard is considered to be present when there is a statistically significant (p<0.05) difference in combined mRNA and miRNA expression between the subject and reference standard or profile.

Thus, in one embodiment, a method for increasing the sensitivity and specificity of an assay for discriminating between subjects with lung cancer and subjects with benign nodules is provided. The method comprises obtaining a biological fluid or tissue sample from a subject; detecting whether one or more mRNA target (e.g., an mRNA target of Table 1, 2 or 3 below) is present in the sample by contacting the sample with at least one ligand selected from a nucleic acid sequence, polynucleotide or oligonucleotide capable of specifically complexing with, hybridizing to, or identifying one or more mRNA gene transcript target of Table 1, 2 or 3 from a mammalian biological sample. Another step of this method involves detecting whether one or more miRNA target (e.g., an miRNA target of Table 1, 2 or 3) is present in the sample by contacting the sample with at least one ligand selected from a nucleic acid sequence, polynucleotide or oligonucleotide capable of specifically complexing with, hybridizing to, or identifying one or more miRNA target of Table 1, 2 or 3 from the same mammalian biological sample. Each ligand used in the method binds to a different mRNA target or miRNA target. In certain embodiments, the combination of detection of both mRNA targets with miRNA targets permits greater sensitivity or specificity or both of diagnosis. In one embodiment, the method permits increased accuracy of identifying whether a subject has a lung cancer or a benign nodule. In another embodiment, the methods increases accuracy of discriminating between a subject with lung cancer and subject who is a smoker without nodules. The smoker may have other symptoms characteristic of a non-cancer disorder. See the examples below.

Table 1 identifies a list of 145 mRNA and miRNAs useful in forming combined mRNA and/or miRNA profiles for use in diagnosing patients with a lung cancer or lung disease from a reference standard, particularly healthy or non-healthy subjects, including subjects with pulmonary disease. This set of 145 mixed sequences is referenced in the comparison of lung cancer vs. patients with nodules (NOD) and smokers without nodules (SC) referenced in Table 5 in the examples below. Table 1 is a list of ranked features (mRNA and miRNA) selected by FFS procedure in Cancer vs Control SVM classifier training. miRNAs are indicated by asterisk. The mRNAs are identified by NCBI accession numbers; the miRNAs are identified by ABI OpenArray identifier numbers (OA #). These sequences are publically available. The SEQ ID Nos for the target sequences correspond with the rank number and are SEQ NO. 1 to 145, respectively. As shown in column 1 of Table 1 (Rank & SEQ ID NO), the rank and SEQ ID NO: are the same number. It should be understood the other target sequences from the mRNAs can be used similarly.

TABLE 1

| Rank & | | | | | |
|---|---|---|---|---|---|
| SEQ ID | | | | | |
| NO: | ID | Type | Accession # | Symbol | Target Sequence |
| 1* | OA_ | miRNA | hsa-miR- | miR-186 | CAAAGAAUUCUCCUUUUGGGCU |
| | 002285 | | 186 | | |
| 2 | ILMN_ | mRNA | NM_ | CBLL1 | GATTGCAGGGTCCGCCTTCTCAAAC |
| | 1705433 | | 024814.1 | | CCCACTTCCTGGACCACATCATCCA |
| 3* | OA_ | miRNA | hsa-miR- | miR-106b | UAAAGUGCUGACAGUGCAGAU |
| | 000442 | | 106b | | |
| 4* | OA_ | miRNA | hsa-miR- | miR-130a | CAGUGCAAUGUUAAAAGGGCAU |
| | 000454 | | 130a | | |
| 5 | ILMN_ | mRNA | NM_ | UBTF | GTCCCAAAGAGTTTGATGAGGCCCT |
| | 1806946 | | 001076683.1 | | CCACACCTGCGGCCCAATCCAAGGT |
| 6* | OA_ | miRNA | hsa-miR- | miR- | UACCACAGGGUAGAACCACGG |
| | 002234 | | 140-3p | 140-3p | |
| 7 | ILMN_ | mRNA | NM_ | CREB1 | TCAACGCCAGGAATCATGAAGAGA |
| | 2382758 | | 134442.2 | | CTTCTGCTTTTCAACCCCCACCCTCC |
| 8 | ILMN_ | mRNA | NM_ | PAMR1 | TCTGGAGGCTGGGAAGTCCAAGAT |
| | 1788410 | | 015430.2 | | CAAGGCGTCAGAAGATTCATTGTCT |
| | | | | | G |
| 9* | OA_ | miRNA | hsa-miR- | miR-21 | UAGCUUAUCAGACUGAUGUUGA |
| | 000397 | | 21 | | |
| 10 | ILMN_ | mRNA | NM_ | SFRS15 | GCCTGAGGTGACAGACAGGGCAGG |
| | 1659874 | | 020706.1 | | TGGTAACAAAACCGTTGAACCTCCC |
| | | | | | A |
| 11 | ILMN_ | mRNA | NM_ | TMEM67 | AAGCTGTTGAAGGTGAGGGTGGTG |
| | 1705049 | | 153704.3 | | TACGAAGTGCCACTGTTCCTGTAAG |
| | | | | | C |
| 12 | ILMN_ | mRNA | NM_ | NCOA5 | AGAAGGAGGGTTTCTGGCTGTGGT |
| | 1770035 | | 020967.2 | | TCTAAATGGAGCCCCAGGAAGCTGC |
| | | | | | C |
| 13 | ILMN_ | mRNA | NM_ | RNF4 | CACTGTCGTCCTTCCTCAGAGGGCC |
| | 1687941 | | 300298.2 | | TCACGCCAAACAAACGGCCTTTTCG |
| 14 | ILMN_ | mRNA | NM_ | WBP11 | GCTAACATCCATTCCCTTTCATACCA |
| | 1766435 | | 016312.2 | | CCATTTTCACCCTGTTTCTTCCCC |
| 15 | ILMN_ | mRNA | NM_ | GOLGA3 | GGTCCAGGTGAATCTCGTCATAAGT |
| | 1733511 | | 900585.3 | | GATCTCAGGCTCTCACAGGATCCGG |
| 16 | ILMN_ | mRNA | XM_ | ZNF807 | AAACTCAAGGACTGCGTGACCGAC |
| | 3299330 | | 944377.3 | | ACAATGACCCCCGAGGAGACAGAG |
| | | | | | GC |
| 17 | ILMN_ | mRNA | NM_ | SIN3A | CCTTGCTGCCTACCCTTTTCTCTCCTC |
| | 1805996 | | 015477.1 | | TGGTTCTCAACCTCAACGAGTTC |
| 18 | ILMN_ | mRNA | NM_ | BTG2 | CCAAACACTCTCCCTACCCATTCCTG |
| | 1770085 | | 006763.2 | | CCAGCTCTGCCTCCTTTTCAACTC |
| 19 | ILMN_ | mRNA | NM_ | EXD2 | CAGGTTGCAATATGAGGACTTCTCT |
| | 1771689 | | 018199.2 | | GTCTCCTCTGAAGCCTGGGACACTG |
| 20 | ILMN_ | mRNA | NM_ | ZNF226 | GAAAGAAATGAGCAGCTTTGGATA |
| | 1692133 | | 001032372.1 | | ATGACGACAGCAACCCGAAGACAG |
| | | | | | GG |
| 21 | ILMN_ | mRNA | NM_ | ADAT2 | GCTCGTGTGCTACAATGGCAGAGTT |
| | 3245693 | | 182503.2 | | GAGCAGTGGTGACAAACCATGCGA |
| | | | | | C |
| 22 | ILMN_ | mRNA | NM_ | FAM171A1 | GCCAAGTGCCATTTGGGGTCAGCAT |
| | 1749868 | | 001010924.1 | | CCTCGTTTCAACACAGTGTGCTCTC |
| 23 | ILMN_ | mRNA | NM_ | ALKBH1 | GTCAGTCCAAGGAGGTATGTTCTTC |
| | 1758038 | | 006020.2 | | CACAACAGCCTTCTCAGCCTCTGCT |
| 24 | ILMN_ | mRNA | NM_ | HNRNPK | CAGAAGAGGGAGACCTGGAGACCG |
| | 3179371 | | 031263.2 | | TTACGACGGCATGGTTGGTTTCAGT |
| | | | | | G |
| 25 | ILMN_ | mRNA | XM_ | LOC642197 | GCTTGCTGCTTTCTGGCTAATGAAA |
| | 1815303 | | 936354.2 | | GCCAAGGACTATCCAGCACACACAG |
| 26 | ILMN_ | mRNA | NM_ | RHOB | GCAGGTCATGCACACAGTTTTGATA |
| | 1802205 | | 004040.2 | | AAGGGCAGTAACAAGTATTGGGGC |
| | | | | | C |
| 27 | ILMN_ | mRNA | NM_ | GSTO1 | GACTGGCAAGGTTTCCTAGAGCTCT |
| | 2227573 | | 004832.1 | | ACTTACAGAACAGCCCTGAGGCCTG |
| 28 | ILMN_ | mRNA | NM_ | HNRPM | GCCTGCCGGATGATGAATGGCATG |
| | 1745385 | | 005968.2 | | AAGCTGAGTGGCCGAGAGATTGAC |
| | | | | | GT |
| 29* | OA_ | miRNA | hsa-miR- | miR-221 | AGCUACAUUGUCUGCUGGGUUUC |
| | 000524 | | 221 | | |
| 30 | ILMN_ | mRNA | XM_ | MARCH14 | TGTCTGTCATTGTGGCCCGTTTCACA |
| | 1671854 | | 944593.1 | | CTGTCTCTATATCTGTTTCCCCTG |
| 31 | ILMN_ | mRNA | NM_ | XPC | AGTCTTCATCTGTCCGACAAGTTCA |
| | 1790807 | | 004628.3 | | CTCGCCTCGGTTGCGGACCTAGGAC |
| 32 | ILMN_ | mRNA | AA789270 | AA789270 | GCCGCCTCGCAAGCTCTTGTTTTCTA |
| | 1890877 | | | | ACCCCACCTTCTGGGAGCCGTGTT |
| 33 | ILMN_ | mRNA | NM_ | NMB | GTCATGATCTGCTCGGAATCCTCCT |
| | 2347592 | | 021077.3 | | GCTAAAGAAGGCTCTGGGCGTGAG |
| | | | | | C |
| 34* | OA_ | miRNA | hsa-miR- | miR-652 | AAUGGCGCCACUAGGGUUGUG |
| | 002352 | | 652 | | |
| 35 | ILMN_ | mRNA | NM_ | IL18BP | GGAGTATGGGAGAGAGGGACTGCC |
| | 1653575 | | 173044.1 | | ACACAGAAGCTGAAGACAACACCT |
| | | | | | GC |
| 36 | ILMN_ | mRNA | NM_ | SAP130 | CCACCCCATTCGGTTCTTCTGCCTGA |
| | 1700044 | | 024545.2 | | CCTTCAAATGCCCATGTTGGCCTT |
| 37* | OA_ | miRNA | hsa-miR- | miR- | UCCGGUUCUCAGGGCUCCACC |
| | 002322 | | 671-3p | 671-3p | |
| 38* | OA_ | miRNA | hsa-miR- | miR- | AAAAGUGCUUACAGUGCAGGUAG |
| | 002169 | | 106a | 106a | |
| 39 | ILMN_ | mRNA | NM_ | FAM102A | AGACTCCTCCAGACCAGGAACCCCA |
| | 1745112 | | 001035254.1 | | GAAGGAGACAGAGCCTGCCACATC |
| | | | | | C |
| 40 | ILMN_ | mRNA | NM_ | GPR65 | CCGGAAAGTCTACCAAGCTGTGCG |
| | 1734740 | | 003608.2 | | GCACAATAAAGCCACGGAAAACAA |
| | | | | | GG |
| 41 | ILMN_ | mRNA | XR_ | LOC648927 | CACCTGTGGGCAGTGGGCAGTGTC |
| | 3274914 | | 038906.1 | | TTGGTGAAAGGGAGCGGATACTAC |
| | | | | | TT |
| 42 | ILMN_ | mRNA | NM_ | ANKRD27 | GAGGCCAGGCTGAAATGTCATATCT |
| | 1794063 | | 032139.2 | | GAAGGAAGAAAGCAGCAGCTGGAC |
| | | | | | A |
| 43 | ILMN_ | mRNA | NM_ | C10orf119 | CAGCGTTAATCCTGTATGGCCAGGA |
| | 1761411 | | 024834.2 | | AACTGAGTAGACTCCTGTGTAACCC |
| 44 | ILMN_ | mRNA | NM_ | OR10X1 | CTGATCTCAGTGTCTGGTTTGCTGG |
| | 1799327 | | 001004477.1 | | GTACCCTTCTGCTCATCATCCTGAC |
| 45* | OA_ | miRNA | hsa-MIR- | MIR-720 | UCUCGCUGGGGCCUCCA |
| | 002895 | | 720 | | |
| 46 | ILMN_ | mRNA | NM_ | PRPF8 | ATCGGGAGGACCTGTATGCCTGACC |
| | 1738677 | | 006445.3 | | GTTTCCCTGCCTCCTGCTTCAGCCT |
| 47 | ILMN_ | mRNA | NM_ | HNRNPD | GGTGACCAGCAGAGTGGTTATGGG |
| | 1751368 | | 002138.3 | | AAGGTATCCAGGCGAGGTGGTCAT |
| | | | | | CA |
| 48 | ILMN_ | mRNA | NM_ | HHLA2 | TAAGATTGCTAGGGAAAAGGGCCC |
| | 1737076 | | 007072.2 | | TATGTGTCAGGCCTCTGAGCCCAAG |
| | | | | | C |
| 49 | ILMN_ | mRNA | NM_ | SPTAN1 | AGCTGCCCTCATTCCGACTTCAGAA |
| | 2095133 | | 003127.1 | | AATCGAAGCAGCTGGCGCCTCCCCT |
| 50* | OA_ | miRNA | hsa-miR- | miR- | GGAUAUCAUCAUAUACUGUAAG |
| | 002148 | | 144_A | 144_A | |
| 51 | ILMN_ | mRNA | NM_ | TRIM32 | CCTCTCGCCTGGAGGATCTGTGCCA |
| | 1654737 | | 012210.3 | | TCTTGGATTGAGAATTGCAGATGTG |
| 52 | ILMN_ | mRNA | XR_ | RNF5P1 | TTCACCATCGTCTTCAATGCCCATGA |
| | 1759948 | | 000528.1 | | GCCTTTCCGCCGGGGTACAGGTGT |
| 53 | ILMN_ | mRNA | BM728869 | BM728869 | GAATCCGATGGTCCTCGAAACATGG |
| | 1857523 | | | | AAAGTCTGCTGTCACGCTGCACGCC |
| 54 | ILMN_ | mRNA | XM_ | LOC651100 | TGCCGGAAGTCACTACCAAGGATCG |
| | 1683920 | | 940229.1 | | ATACACATTTAGGAAAGCCAGCACT |
| 55* | OA_ | miRNA | hsa-miR- | miR-20b | CAAAGUGCUCAUAGUGCAGGUAG |
| | 001014 | | 20b | | |
| 56 | ILMN_ | mRNA | NM_ | HRB | GGAGAGGGTGACCTGGCTGCTGGT |
| | 2196734 | | 004504.3 | | TTACCACTGTACCAACATCTCTGGA |
| | | | | | G |
| 57 | ILMN_ | mRNA | NM_ | EIF4ENI | GGGCTTTTACTTTGGAGCACTCTGT |
| | 1794967 | | 019843.2 | F1 | GTGAAGCTGTTTGGTGGAACCCATG |
| 58 | ILMN_ | mRNA | NM_ | CCR6 | GAGGAGCTGCAGATTAGCTAGGGG |
| | 1690907 | | 031409.3 | | ACAGCTGGAATTATGCTGGCTTCTG |
| | | | | | A |
| 59 | ILMN_ | mRNA | NM_ | CCR4 | CCTGAACTGATGGGTTTCTCCAGAG |
| | 2086143 | | 005508.4 | | GGAATTGCAGAGTACTGGCTGATG |
| | | | | | G |
| 60* | OA_ | miRNA | hsa-miR- | miR-345 | GCUGACUCCUAGUCCAGGGCUC |
| | 002186 | | 345 | | |
| 61 | ILMN_ | mRNA | NM_ | GEMIN4 | GCTTCTTACCTGTGCGGGAGCGAAA |
| | 1770206 | | 015721.2 | | AAGCTGGGCTTCAACATGGCAGGTC |
| 62* | OA_ | miRNA | hsa-let-7e | let-7e | UGAGGUAGGAGGUUGUAUAGUU |
| | 002406 | | | | |
| 63 | ILMN_ | mRNA | NM_ | LPCAT4 | CACTCTATGGGAAACTCTTCAGCAC |
| | 1674759 | | 153613.2 | | CTACCTGCGCCCCCCACACACCTCT |
| 64 | ILMN_ | mRNA | NM_ | C11orf58 | CCCAGCCCTAGATGTATCCAAGCCC |
| | 3250798 | | 001142705.1 | | TCCTACCCTCACCAGTTATTTCTGG |
| 65 | ILMN_ | mRNA | XM_ | LOC100132782 | CTCCAAATGTCAAAGGCAAGCTGG |
| | 3243562 | | 001715620.1 | | GCATCATGATCTGGCATAAAGAACC |
| | | | | | C |
| 66 | ILMN_ | mRNA | NM_ | RAH | GCCCAGGGCCGCCCTAGCAACTTCC |
| | 2060770 | | 030665.3 | | TGTACATATGACTGTAAAATGGTAA |
| 67* | OA_ | miRNA | hsa-miR- | miR-20a | UAAAGUGCUUAUAGUGCAGGUAG |
| | 000580 | | 20a | | |
| 68 | ILMN_ | mRNA | NM_ | C19orf29 | CCCCGAGTTTTGCCCATATCAGGAC |
| | 2262462 | | 001080543.1 | | AGTGGCTCCTTCTCACTCCCCTTTC |
| 69 | ILMN_ | mRNA | NM_ | RBM14 | GCGGCACAGTCCCACTTCCCCATCT |
| | 1700604 | | 006328.2 | | CCCCAAGTAGGTGGTGTTAGAAAAC |
| 70 | ILMN_ | mRNA | XM_ | LOC100132032 | GAAAGCGGCCTCATGAAGGGGAAG |
| | 3290340 | | 001726273.1 | | CCAAGGGTGCCGAGACCACAAAGC |
| | | | | | GC |
| 71 | ILMN_ | mRNA | XM_ | LOC647806 | AGTCGTCCTTCCCTGGTGCGCAGCC |
| | 1812482 | | 943033.1 | | CAGGCCTGTGGGTCCAGCCTCACCC |
| 72 | ILMN_ | mRNA | NM_ | SF3B2 | ATGGCCATGACCCAGAAGTATGAG |
| | 1775939 | | 006842.2 | | GAGCATGTGCGGGAGCAGCAGGCT |
| | | | | | CA |
| 73 | ILMN_ | mRNA | XR_ | LOC100131507 | GCCTGAGGGACCGCAGACTCGTCG |
| | 3288731 | | 038156.1 | | GGCTGCTTTCTGATGAGAGGATTAA |
| | | | | | C |
| 74 | ILMN_ | mRNA | XM_ | LOC642197 | GGAAAGTGAAGATGCAGAGTTACT |
| | 1783469 | | 936354.2 | | GTGGCGTTTGGCACGGGCATCACG |
| | | | | | TG |
| 75 | ILMN_ | mRNA | XM_ | LOC286297 | ACCGATCTTTCTCTGTCTCACCAACC |
| | 1682126 | | 372109.3 | | TGACAAAAAAGGTGTGCCAAGGGA |
| 76 | ILMN_ | mRNA | DQ286431 | DQ286431 | ACGATGCCAGACTCATGTTTGGAGA |
| | 1902146 | | | | TGGAACTCAGCTGGTGGTGAAGCC |
| | | | | | C |
| 77 | ILMN_ | mRNA | XM_ | LOC100130522 | CCTCAAGGAGATGCCTCTGGTCCAG |
| | 3230723 | | 001714664.1 | | GCTTTGTAAACTTGGGCCTTCCAGC |
| 78 | ILMN_ | mRNA | NM_ | FAM43A | GTAGCACTGTTCTGGTTCTGTTTGC |
| | 1706015 | | 153690.4 | | ACGCCAGTGGGGAGAGAATAAAGA |
| | | | | | G |
| 79 | ILMN_ | mRNA | XR_ | LOC100133213 | GGGCAGTACAGGGCCAGATCCACG |
| | 3295894 | | 037788.1 | | GCAGGCACAGGGCAAAGCCAGGCC |
| | | | | | CA |
| 80 | ILMN_ | mRNA | NM_ | TBC1D12 | CCAAGGAATGCACTAAGCCTTCAGT |
| | 1743324 | | 015188.1 | | CTTTTTAGACTGACAGTACTGGCAG |
| 81 | ILMN_ | mRNA | NM_ | KRT75 | CTATACCCATTCCCAGGCCTAAGCC |
| | 1721247 | | 004693.2 | | AGCCTCTCCCTCCTGACAGTGCCCA |
| 82 | ILMN_ | mRNA | NM_ | TSPYL1 | GAGGCATGGGCCAGGTAAAAATTG |
| | 1779014 | | 003309.2 | | GGCCTAGAGTGAAGACTGTGCTGT |
| | | | | | CG |
| 83 | ILMN_ | mRNA | NM_ | B4GALNT1 | GGCTGGGGTGAGGGCTGGTGGTTG |
| | 1805725 | | 001478.3 | | GTGAAAGCCATTCTTAGTTGTGTCT |
| | | | | | C |
| 84* | OA_ | miRNA | hsa-miR- | miR-363 | AAUUGCACGGUAUCCAUCUGUA |
| | 001271 | | 363 | | |
| 85 | ILMN_ | mRNA | NM_ | TPR | GTCAGATCTCCCCTCCACCAGCCAG |
| | 1730999 | | 003292.2 | | GATCCTCCTTCTAGCTCATCTGTAG |
| 86 | ILMN_ | mRNA | NM_ | FLJ45256 | GTGAGCCAAAATGGCGCTACTGCAC |
| | 2149952 | | 207448.1 | | TCCAGACCGGGGACAGAGTGAGAC |
| | | | | | T |
| 87* | OA_ | miRNA | hsa-MIR- | MIR- | GUCCCUGUUCAGGCGCCA |
| | 002883 | | 1274A | 1274A | |
| 88 | ILMN_ | mRNA | NM_ | LRRTM4 | AGGAGAGAGGTTTGAGTTCTGGGT |
| | 1685472 | | 024993.3 | | ATCCTCCCTTTCTGTAACAGCCTCAA |
| 89* | OA_ | miRNA | hsa-miR- | miR- | CAUUAUUACUUUUGGUACGCG |
| | 000451 | | 126_A | 126_A | |
| 90 | ILMN_ | mRNA | NM_ | MED12 | CTTTGGTCCGGCAACTTCAACAACA |
| | 1793386 | | 005120.1 | | GCTCTCTAATACCCAGCCACAGCCC |
| 91 | ILMN_ | mRNA | XM_ | LOC642441 | CCAGCCATCCCATTACTGGGTAGGT |
| | 3248595 | | 930678.3 | | ACCCAAATCATGCTGCTATAAAGAC |
| 92 | ILMN_ | mRNA | | PAF1 | CCCAGGGCATTCAGGGCTGGTTCA |
| | 1669508 | | NM_0190 | | GACACCATTATTGTGAGCAGCAAAG |
| | | | | | C |
| 93 | ILMN_ | mRNA | NM_ | C6orf126 | CCGCCGGTGCCATATGATTTAGAGG |
| | 2054121 | | 207409.1 | | AAGATGCAGGCTGGTCACTGCTCCC |
| 94 | ILMN_ | mRNA | NM_ | SERPINB10 | TCAAGTCAACCCTGAGCAGTATGGG |
| | 2147424 | | 005024.1 | | GATGAGTGATGCCTTCAGCCAAAGC |
| 95 | ILMN_ | mRNA | XM_ | LOC642132 | CATACCACCCTTTGGTGGGAGGAAA |
| | 1791084 | | 936279.2 | | CTAAAAATATAGCAAATGCAGAACC |
| 96* | OA_ | miRNA | hsa-miR- | miR- | CCUGUUCUCCAUUACUUGGCUC |
| | 002444 | | 26b_A | 26b_A | |
| 97 | ILMN_ | mRNA | NM_ | PREI3 | CTAGACGCTGGCACTATGGTCATGG |
| | 1813594 | | 015387.2 | | CGGAGGGGACGGCAGTGCTGAGG |
| | | | | | CG |
| 98 | ILMN_ | mRNA | XM_ | LOC642782 | CTTTTCGCAGATGCTGGGAACGCAG |
| | 1690689 | | 931704.1 | | CTCTGCTGCCGGCGGGGTGGACAG |
| | | | | | A |
| 99 | ILMN_ | mRNA | NM_ | MYL6 | TCGTCCGCATGGTGCTGAATGGCTG |
| | 1809013 | | 021019.3 | | AGGACCTTCCCAGTCTCCCCAGAGT |
| 100 | ILMN_ | mRNA | NM_ | NMNAT2 | GGATCCACATGGTCTTGAGGGTTG |
| | 1803818 | | 015039.2 | | GCATGAGGAGGGGGAAGCTTTTTT |
| | | | | | GA |
| 101 | ILMN_ | mRNA | NM_ | HSP90AB1 | AATGCTGCAGTTCCTGATGAGATCC |
| | 1673711 | | 500735.2 | | CCCCTCTCGAGGGCGATGAGGATG |
| | | | | | C |
| 102 | ILMN_ | mRNA | NM_ | LOC401152 | GTGGTAGATCACTTGAGGTCAAGA |
| | 2051684 | | 001001701.1 | | GTTGTGACACCAGCCTGGCCAACCT |
| | | | | | G |
| 103 | ILMN_ | mRNA | XM_ | LOC650518 | CAAATATCATGGAGGTCCCTGGATT |
| | 1675852 | | 937285.1 | | GAAAAAAGAGCCTCTCCCACTCCTC |
| 104 | ILMN_ | mRNA | NM_ | MRPL49 | CCCTGCCCCCAAACTGGCTAAGACA |
| | 1681324 | | 004927.2 | | GCTTTCAGTTCCTGACTCCCCAACT |
| 105 | ILMN_ | mRNA | NM_ | PUM1 | CTGAGACGGGCAAGTGGTTGCTCC |
| | 2401155 | | 001020658.1 | | AGGATTACTCCCTCCTCCAAAAAAG |
| | | | | | G |
| 106 | ILMN_ | mRNA | NM_ | Cl7orf28 | CTCTGGCCTCTGGGTCCCACCACCC |
| | 1654013 | | 030630.1 | | AGCCCCCCGTGTCAGAACAATCTTT |
| 107 | ILMN_ | mRNA | NM_ | ZNF239 | TCCTCGCTAACTGACATTAGCCCATT |
| | 1748427 | | 001099283.1 | | CAGGTCTTCACAGCGCTCATACTG |
| 108 | ILMN_ | mRNA | NM_ | ID3 | CCCCAACTTCGCCCTGCCCACTTGAC |
| | 1732296 | | 002167.2 | | TTCACCAAATCCCTTCCTGGAGAC |
| 109 | ILMN_ | mRNA | NM_ | ARHGEF2 | TGGGGGATTTTTCAGTGGAACCCTT |
| | 1703477 | | 004723.2 | | GCCCCCAAATGTCGACCAGCCCCCA |
| 110 | ILMN_ | mRNA | NM_ | IL13RA2 | GTAACCGGTCTGCTTTTGCGTAAGC |
| | 1688722 | | 000640.2 | | CAAACACCTACCCAAAAATGATTCC |
| 111 | ILMN_ | mRNA | NM_ | SFT2D2 | GGCCAGTTTTATGAAGCTTTGGAAG |
| | 3307659 | | 199344.2 | | GCACTATGGACAGAAGCTGGTGGA |
| | | | | | C |
| 112 | ILMN_ | mRNA | NM_ | P2RY8 | CTATGGAGAGCAGCCGACACCCCCT |
| | 1768284 | | 178129.3 | | CTTACAGCCGTGGATGTTTCCTGGA |
| 113 | ILMN_ | mRNA | NM_ | PCDHA12 | GGCCACGGTGCTGGTGTCGCTGGT |
| | 2338687 | | 031864.1 | | GGAGAACGGCCAGGCCCCAAAGAC |
| | | | | | GT |
| 114 | ILMN_ | mRNA | NM_ | FMNL2 | AGTGTACCTATTTACAGAAAGATTA |
| | 1730491 | | 052905.3 | | AACTGCCACCTGCGGGCACATTCCC |
| 115 | ILMN_ | mRNA | NM_ | KRT32 | TACTGAAGTCCCTTTGTGCCAGTGG |
| | 1807249 | | 002278.3 | | ATCCTGGAGGGCCTGGGGCTGGGC |
| | | | | | A |
| 116 | ILMN_ | mRNA | NM_ | RPS21 | CGCCGATATCTCTGCCGGGTGACTA |
| | 1800573 | | 001024.3 | | GCTGCTTCCTTTCTCTCTCGCGCGC |
| 117 | ILMN_ | mRNA | NM_ | RNF34 | CGACTGCCAGGGCCTTAGACTCCAC |
| | 1786039 | | 025126.2 | | ATGTCCATTTTTGTTCAGGTATAGC |
| 118 | ILMN_ | mRNA | NM_ | FAIM3 | CTCGGGCATCCTTCCCAGGGTTGGG |
| | 1775542 | | 005449.3 | | TCTTACACAAATAGAAGGCTCTTGC |
| 119* | OA_ | miRNA | hsa-miR- | miR-340 | UUAUAAAGCAAUGAGACUGAUU |
| | 002258 | | 340 | | |
| 120 | ILMN_ | mRNA | XM_ | FLJ43950 | CCACAGCCTGTTTCTCCCTTGGATTC |
| | 1718657 | | 001127087.1 | | CAAGTTCCCCATAGACCATTCCCT |
| 121 | ILMN_ | mRNA | BG201089 | BG201089 | CCCTCAACTGCCTTTCCACCACCTAT |
| | 1852756 | | | | GATGTTGGGGTTTCAGAAAAGGTG |
| 122 | ILMN_ | mRNA | NM_ | GTF3C2 | CCACAGACACCCTACCGATAGAACA |
| | 1746457 | | 001521.2 | | GTGGCTCAGATCTTACTTGCTCCTG |
| 123 | ILMN_ | mRNA | NM_ | IP6K2 | TACGAGACCCTCCCTGCTGAGATGC |
| | 1683328 | | 001005910.1 | | GCAAATTCACTCCCCAGTACAAAGG |
| 124 | ILMN_ | mRNA | NM_ | NAT10 | GTGCTGTTCCACTCTTGGCTCCAGC |
| | 1705594 | | 024662.1 | | AGACCCACTGTCCCAGAAAAGCCTG |
| 125 | ILMN_ | mRNA | NM_ | IFI27L2 | CCCAGCTGAACCCGAGGCTAAAGA |
| | 1740319 | | 032036.2 | | AGATGAGGCAAGAGAAAATGTACC |
| | | | | | CC |
| 126 | ILMN_ | mRNA | NM_ | EIF4A3 | CAGCAGATCAGTGGGATGAGGGAG |
| | 1667043 | | 014740.2 | | ACTGTTCACCTGCTGTGTACTCCTGT |
| 127 | ILMN_ | mRNA | NM_ | ARHGEF18 | CGTGGGATCTGCACACGTCTTTGTC |
| | 1664016 | | 101538.2 | | AGTTGTGGTCATGATCTTAGTCACC |
| 128 | ILMN_ | mRNA | NM_ | SFMBT1 | GGAGTGTGGCAGACGTTGTGCGGT |
| | 2391750 | | 001005158.1 | | TCATCAGATCCACTGACTGTGCTCC |
| | | | | | A |
| 129 | ILMN_ | mRNA | NM_ | C11orf44 | TCTGCTGGACTGATGTCTTCTGCAG |
| | 1803015 | | 173580.1 | | GTTGCAGATCCTGACCATGGGCTGC |
| 130 | ILMN_ | mRNA | NM_ | DYNLT1 | CGTCAGTGCCTTCGGACTGTCTATTT |
| | 1678766 | | 006519.1 | | GACCTGCAGTCCAGCCTATGGCCT |
| 131 | ILMN_ | mRNA | AW026064 | AW026064 | ACTTGTCCACGGTCCTCTCGGTGAC |
| | 1908133 | | | | CCTGTTGGGCAGGGCCAAGGGACA |
| | | | | | A |
| 132 | ILMN_ | mRNA | NM_ | CASP14 | CGCCTACCGACATGATCAGAAAGGC |
| | 1739513 | | 101214.1 | | TCATGCTTTATCCAGACCCTGGTGG |
| 133 | ILMN_ | mRNA | NM_ | CKLF | ACATCGCCCCTTCTGCTTCAGTGTG |
| | 1712389 | | 001040138.1 | | AAAGGCCACGTGAAGATGCTGCGG |
| | | | | | C |
| 134 | ILMN_ | mRNA | NM_ | MARCKSL1 | CCTGAGCCAGAAGTGGGGTGCTTA |
| | 1714433 | | 023009.4 | | TACTCCCAAACCTTGAGTGTCCAGC |
| | | | | | C |
| 135 | ILMN_ | mRNA | XM_ | LOC644477 | AAATTGAACACAAATGTGGTGGAG |
| | 1707954 | | 942968.1 | | ACGGGACAGGGCAGGTGGAAATTC |
| | | | | | AC |
| 136 | ILMN_ | mRNA | NM_ | TCF7 | GGCAGAGAAGGAGGCCAAGAAGC |
| | 2367141 | | 201632.1 | | CAACCATCAAGAAGCCCCTCAATGC |
| | | | | | CT |
| 137 | ILMN_ | mRNA | NM_ | TMEM14E | CCCAGGCTGGTCTTACAGCCTCAGG |
| | 3236468 | | 001123228.1 | | CAATCCTCTGGTCTTGACGTCCCAA |
| 138 | ILMN_ | mRNA | XM_ | LOC100131801 | AGGCCGAGTGGTTTGAGGACGATG |
| | 3209832 | | 001726504.1 | | TCATACAGCGCAAGAGGGAGCTGT |
| | | | | | GG |
| 139 | ILMN_ | mRNA | NM_ | S100A8 | TAACTTCCAGGAGTTCCTCATTCTG |
| | 1729801 | | 002964.3 | | GTGATAAAGATGGGCGTGGCAGCC |
| | | | | | C |
| 140 | ILMN_ | mRNA | NM_ | MEOX2 | CTTCCTGATTGACAACAGTGTTAGA |
| | 1777263 | | 005924.4 | | CAAGGTGCAAAGCGAAACTGGTTG |
| | | | | | C |
| 141 | ILMN_ | mRNA | NM_ | SF3A1 | AGTGCTCCTGTTGCAGGACTGCTGG |
| | 1697286 | | 001005409.1 | | GAAAACAGGTGGTGTGGGACTTAA |
| | | | | | G |
| 142 | ILMN_ | mRNA | NM_ | MAGED1 | CAGCCAGTGCCAACTTCGCTGCCAA |
| | 1775522 | | 001005332.1 | | CTTTGGTGCCATTGGTTTCTTCTGG |
| 143 | ILMN_ | mRNA | NM_ | TRIM28 | GAAGTTGTCACCTCCCTACAGCTCC |
| | 1736575 | | 005762.2 | | CCACAGGAGTTTGCCCAGGATGTG |
| | | | | | G |
| 144 | ILMN_ | mRNA | NM_ | DDX47 | ACAGCTTTGCTACTGCGAAATCTTG |
| | 1747162 | | 501635.3 | | GCTTCACTGCCATCCCCCTCCATGG |
| 145 | ILMN_ | mRNA | XM_ | LOC648615 | CCCCACCCCCGCGTTCCGACCGCTG |
| | 1811346 | | 937684.1 | | AAGCTCCAAATTCAGGCCTTAAATA |

Table 2 identifies a list of about 147 mRNA and miRNAs useful in forming combined mRNA and/or miRNA profiles for use in diagnosing patients with a lung cancer or lung disease from a reference standard, particularly healthy or non-healthy subjects, including subjects with pulmonary disease. This set of 147 mixed sequences is referenced in the comparison of lung cancer vs. patients with nodules (NOD) referenced in Table 5 in the examples below. Table 2 is a list of ranked features (mRNA and miRNA) selected by FFS procedure in Cancer vs Control SVM classifier training. The mRNAs are identified by NCBI accession numbers; the miRNAs are identified by ABI OpenArray identifier numbers (OA #). The target sequences used in the examples below are provided in the Table below. However other portions of the sequences identified by the accession numbers can also be used in a similar manner. These sequences are publically available. The SEQ ID Nos for the target sequences 1-147 in Table 2 are SEQ NO. 146 to 292, respectively and are identified in column Rank/SEQ ID No. These sequences are publically available.

TABLE 2

| Rank/Seq | | | Accession | | |
|---|---|---|---|---|---|
| ID No. | ID | Type | # | Symbol | Target Sequence |
| 1/146 | OA_ | miRNA | hsa-let-7d | let-7d | AGAGGUAGUAGGUUGCAUAGU |
| | 002283 | | | | U |
| 2/147 | OA_ | miRNA | hsa-miR- | miR-186 | CAAAGAAUUCUCCUUUUGGGCU |
| | 002285 | | 186 | | |
| 3/148 | ILMN_ | mRNA | NM_ | DNAJB1 | CATTTCTGTAAGGCAATCTTGGCA |
| | 1775304 | | 006145.1 | | CACGTGGGGCTTACCAGTGGCCC |
| | | | | | AGG |
| 4/149 | ILMN_ | mRNA | NM_ | TP53BP1 | CCTGTGCCTTGCCAGTGGGATTCC |
| | 1664440 | | 005657.1 | | TTGTGTGTCTCATGTCTGGGTCCA |
| | | | | | TG |
| 5/150 | ILMN_ | mRNA | NM_ | GSTO1 | GAAGCATACCCAGGGAAGAAGCT |
| | 1808196 | | 004832.1 | | GTTGCCGGATGACCCCTATGAGA |
| | | | | | AAGC |
| 6/151 | OA_ | miRNA | hsa-miR- | miR- | UAAAGUGCUGACAGUGCAGAU |
| | 000442 | | 106b | 106b | |
| 7/152 | ILMN_ | mRNA | NM_ | HERC1 | CGACACTGACTACTGACCGTGCG |
| | 1786211 | | 003922.3 | | GGTGCTCTCACCCTCCCTTCTCTCC |
| | | | | | CT |
| 8/153 | ILMN_ | mRNA | XM_ | LOC652615 | TCTGTGCCCTTTATCCGCACTTCCC |
| | 1773797 | | 942150.1 | | AGCTCACAGCACTGACAACCGGT |
| | | | | | GA |
| 9/154 | ILMN_ | mRNA | NM_ | EIF4H | GCACCCAGCGGAATGTGCTTAGT |
| | 2304624 | | 022170.1 | | ATTTGGTCACCAGCCGTCATCCTG |
| | | | | | GGC |
| 10/155 | ILMN_ | mRNA | NM_ | HNRNPK | CAGAAGAGGGAGACCTGGAGAC |
| | 3179371 | | 031263.2 | | CGTTACGACGGCATGGTTGGTTT |
| | | | | | CAGTG |
| 11/156 | ILMN_ | mRNA | NM_ | MLL5 | GCATCTCCAGTGCCTGGACAGATT |
| | 1783606 | | 018682.3 | | CCAATTCACAGAGCACAGGTGCC |
| | | | | | ACC |
| 12/157 | ILMN_ | mRNA | NM_ | GSTO1 | GACTGGCAAGGTTTCCTAGAGCT |
| | 2227573 | | 004832.1 | | CTACTTACAGAACAGCCCTGAGG |
| | | | | | CCTG |
| 13/158 | OA_ | miRNA | hsa-miR- | miR-18a | UAAGGUGCAUCUAGUGCAGAUA |
| | 002422 | | 18a | | G |
| 14/159 | ILMN_ | mRNA | NM_ | CREB1 | TCAACGCCAGGAATCATGAAGAG |
| | 2382758 | | 134442.2 | | ACTTCTGCTTTTCAACCCCCACCCT |
| | CC | | | | |
| 15/160 | ILMN_ | mRNA | NM_ | NDUFV2 | GCTCAAGGCTGGCAAAATCCCAA |
| | 2086417 | | 021074.1 | | AACCAGGGCCAAGGAGTGGACG |
| | | | | | CTTCT |
| 16/161 | ILMN_ | mRNA | NM_ | CABC1 | GGCTGGAGCTGGGAGAGGTGCT |
| | 1731064 | | 020247.4 | | GAGCTAACAGTGCCAACAAGTGC |
| | | | | | TCCTT |
| 17/162 | ILMN_ | mRNA | NM_ | SIN3A | CCTTGCTGCCTACCCTTTTCTCTCC |
| | 1805996 | | 015477.1 | | TCTGGTTCTCAACCTCAACGAGTT |
| | | | | | C |
| 18/163 | ILMN_ | mRNA | XR_ | LOC729852 | GGCAGTACAGGGCACCATCACTG |
| | 3222425 | | 040870.1 | | ACCTTCCCGACCACTTACTCTCCTA |
| | | | | | TG |
| 19/164 | ILMN_ | mRNA | NM_ | MBD1 | GGATGGCCTGGAACCCATGTCAG |
| | 2352580 | | 015844.1 | | TCTCTCACCACCTCCAGCTTCGAT |
| | | | | | GAT |
| 20/165 | ILMN_ | mRNA | NM_ | KLF13 | TTGCTTGTGTGCATGTGTTGGGTG |
| | 1679929 | | 015995.2 | | CATGCTTCCGGGTCTCAGCTGCCC |
| | | | | | CA |
| 21/166 | OA_ | miRNA | hsa-miR- | miR- | UGAGCGCCUCGACGACAGAGCC |
| | 002184 | | 339-3p | 339-3p | G |
| 22/167 | ILMN_ | mRNA | NM_ | PCDHGB5 | GGGCCTTATTTCCACTTTGTAATT |
| | 1811103 | | 018925.2 | | CCAGCGAGTCGACTTCCCATCCTG |
| | | | | | AG |
| 23/168 | ILMN_ | mRNA | XM_ | LOC652554 | ACTTAAAAAATACTTCGTTTATCA |
| | 1772147 | | 942053.2 | | CATCTCAGGAACTAAACTGGGTT |
| | | | | | AAG |
| 24/169 | ILMN_ | mRNA | NM_ | RCSD1 | TGCAAGGGACAGGGGGCCTGACT |
| | 1749006 | | 052862.2 | | ACCCAGTCTTTGACTTGTATCCTC |
| | | | | | TCC |
| 25/170 | ILMN_ | mRNA | NM_ | STOM | TCACTTGGGAGGGACGCATAGAA |
| | 1766657 | | 004099.4 | | GGAGCTCTAGGAACACAGTGCCA |
| | | | | | GTGC |
| 26/171 | ILMN_ | mRNA | NM_ | RBM16 | GTGCCTCAGGTTAATGGTGAAAA |
| | 1681675 | | 014892.3 | | TACAGAGAGACATGCTCAGCCAC |
| | | | | | CACC |
| 27/172 | ILMN_ | mRNA | NM_ | SETD2 | GACCTGACTCCACTCTTAAACCTG |
| | 1769473 | | 014159.4 | | GGTCTTCTCCTTGGCGGTGCTGTC |
| | | | | | AG |
| 28/173 | ILMN_ | mRNA | NM_ | ATP5E | TCTGATCTTCCTGCGGCTGAACCG |
| | 3261197 | | 001001977.1 | | CCCGGCTGAGCCGACATTGCCGG |
| | | | | | CGT |
| 29/174 | ILMN_ | mRNA | NM_ | PIK3R5 | TGAGGCTCTGGTGCTCAGGGGGA |
| | 1681067 | | 014308.2 | | TGGCTTGGGCCTTTTCTCTCAACC |
| | | | | | TTG |
| 30/175 | ILMN_ | mRNA | NM_ | CHERP | ATCCAGAGCATGGAGCCCGACCC |
| | 1798083 | | 006387.5 | | CAGCCAGCGCCTTCCACTCCATCA |
| | | | | | TTT |
| 31/176 | ILMN_ | mRNA | NM_ | TCF20 | GAGGGACTGTCGCTGTGATCAGA |
| | 2368068 | | 181492.1 | | GTGGGTTAAGCTGACCAGGAACA |
| | | | | | CCCA |
| 32/177 | ILMN_ | mRNA | NM_ | DNAJB6 | CCGAGGGACGGGGTCGTTTTTCT |
| | 2402416 | | 005494.2 | | CTGCGTTCAGTGGATTTCCGTCTT |
| | | | | | TTG |
| 33/178 | ILMN_ | mRNA | NM_ | MBD1 | AGGATGGCCTGGAACCCATGTCA |
| | 1683595 | | 015845.2 | | GTCTCTCACCACCTCCAGCTTCGA |
| | | | | | TGA |
| 34/179 | OA_ | miRNA | hsa-let-7a | let-7a | UGAGGUAGUAGGUUGUAUAGU |
| | 000377 | | | | U |
| 35/180 | ILMN_ | mRNA | N_1389 | SON | GCTAAGGCTGGTGTCCCTTTACCA |
| | 1703427 | | 27.1 | | CCAAACCTAAAGCCTGCACCTCCA |
| | | | | | CC |
| 36/181 | ILMN_ | mRNA | NM_ | SEC24C | CTCTCCTGCTGGGACACCGCTTGG |
| | 1676600 | | 198597.1 | | GCTTTGGTATTGACTGAGTGGCT |
| | | | | | GAC |
| 37/182 | ILMN_ | mRNA | NM_ | PHF3 | GTGCTCTGTACCAGTGCTCATCAT |
| | 1798164 | | 015153.1 | | CCCTTCTTCATACCAACGGTCCCT |
| | | | | | AG |
| 38/183 | ILMN_ | mRNA | NM_ | ATP5J2 | CTTGGCCCGAGCCCCTCCGTGAG |
| | 2307883 | | 001003714.1 | | GAACACAATCTCAATCGTTGCTGA |
| | | | | | ATC |
| 39/184 | ILMN_ | mRNA | NM_ | RNF214 | CCTGCTCCACTGGCCCAAATCAGT |
| | 1800420 | | 207343.2 | | ACCCCAATGTTCTTGCCTTCTGCC |
| | | | | | CA |
| 40/185 | ILMN_ | mRNA | NM_ | PLAC8 | TAAGGCCCTGCACTGAAAATGCA |
| | 1653026 | | 016619.1 | | AGCTCAGGCGCCGGTGGTCGTTG |
| | | | | | TGAC |
| 41/186 | ILMN_ | mRNA | NM_ | UNC119B | CCAGTGTCACTATGATGTCAGTGA |
| | 3245351 | | 001080533.1 | | GGTCTGGGGATGAGGACAGTGT |
| | | | | | GTCC |
| 42/187 | ILMN_ | mRNA | NM_ | NUP153 | CACTGATTTGACATAGTCTGGCTG |
| | 1705907 | | 005124.2 | | TACCCAGGAATGGAGCCTGCACG |
| | | | | | GTG |
| 43/188 | ILMN_ | mRNA | NM_ | TPR | GTCAGATCTCCCCTCCACCAGCCA |
| | 1730999 | | 003292.2 | | GGATCCTCCTTCTAGCTCATCTGT |
| | | | | | AG |
| 44/189 | ILMN_ | mRNA | XR_ | LOC92755 | ATCGAGTCCTACAATGCTACCCTC |
| | 3304898 | | 016140.2 | | TCCGTCCATCAGTTGGTAGAGAA |
| | | | | | CAC |
| 45/190 | ILMN_ | mRNA | NM_ | RHAG | GCTGGAACCTGAAGTCTAAACAC |
| | 1811410 | | 000324.1 | | CATTCCTGCTCTCCAGCTTCCTTTC |
| | | | | | CC |
| 46/191 | ILMN_ | mRNA | NM_ | STK38 | CTGCAGCTGGGAGCCTGCTTTCT |
| | 2152581 | | 007271.2 | | GCCAGTCTTGAGGTTCTGAAGAT |
| | | | | | CAGC |
| 47/192 | ILMN_ | mRNA | NM_ | LRRC47 | CTGTACAGTCATGTGCCACGTAAC |
| | 1668484 | | 020710.1 | | AGCGTCTGGGTCAGTGACGGACA |
| | | | | | CTT |
| 48/193 | ILMN_ | mRNA | NM_ | M54A4A | TCCCTGGAACTCAATAACTCATTT |
| | 2370336 | | 148975.1 | | CACTGGCTCTTTATCGAGAGTACT |
| | | | | | AG |
| 49/194 | ILMN_ | mRNA | NM_ | RNF114 | GTCTGGAGGGAAATCTGGCGAAA |
| | 1792078 | | 018683.3 | | CCTTCGTTTGAGGGACTGATGTG |
| | | | | | AGTG |
| 50/195 | ILMN_ | mRNA | XR_ | LOC648927 | CACCTGTGGGCAGTGGGCAGTGT |
| | 3274914 | | 038906.1 | | CTTGGTGAAAGGGAGCGGATACT |
| | | | | | ACTT |
| 51/196 | ILMN_ | mRNA | NM_ | SGK1 | CGGACGCTGTTCTAAAAAAGGTC |
| | 3229324 | | 005627.3 | | TCCTGCAGATCTGTCTGGGCTGTG |
| | | | | | ATG |
| 52/197 | ILMN_ | mRNA | NM_ | JAK1 | ATTGCCTCTGACGTCTGGTCTTTT |
| | 1793384 | | 002227.2 | | GGAGTCACTCTGCATGAGCTGCT |
| | | | | | GAC |
| 53/198 | ILMN_ | mRNA | NM_ | NDRG2 | GCTGAGGGGTAAGAGGTTGTTGT |
| | 2361603 | | 201539.1 | | AGTTGTCCTGGTGCCTCCATCAGA |
| | | | | | CTC |
| 54/199 | ILMN_ | mRNA | NM_ | RNASE2 | GGAAGCCAGGTGCCTTTAATCCA |
| | 1730628 | | 002934.2 | | CTGTAACCTCACAACTCCAAGTCC |
| | | | | | ACA |
| 55/200 | ILMN_ | mRNA | NM_ | MYH9 | CTAGGACTGGGCCCGAGGGTGGT |
| | 1722872 | | 002473.3 | | TTACCTGCACCGTTGACTCAGTAT |
| | | | | | AGT |
| 56/201 | ILMN_ | mRNA | NM_ | GNB1 | TTCCGTCCAACAACTCTGTAGAGC |
| | 1760320 | | 002074.2 | | TCTCTGCACCCTTACCCCTTTCCAC |
| | | | | | C |
| 57/202 | ILMN_ | mRNA | NM_ | WDR1 | CATACCGGCTGGCCACGGGAAGC |
| | 1675844 | | 017491.3 | | GATGATAACTGCGCGGCATTCTTT |
| | | | | | GAG |
| 58/203 | ILMN_ | mRNA | NM_ | RASSF5 | GCTCCTGCTGCAACCGCTGTGAAT |
| | 2362902 | | 182664.2 | | GCTGCTGAGAACCTCCCTCTATGG |
| | | | | | GG |
| 59/204 | ILMN_ | mRNA | NM_ | SMARCC1 | CCCCTGGAGTCCGAGAAGGAAAA |
| | 1694603 | | 003074.2 | | TGGAATTCTGGTTCATACTGTGGT |
| | | | | | CCC |
| 60/205 | ILMN_ | mRNA | NM_ | C6orf126 | CCGCCGGTGCCATATGATTTAGA |
| | 2054121 | | 207409.1 | | GGAAGATGCAGGCTGGTCACTGC |
| | | | | | TCCC |
| 61/206 | ILMN_ | mRNA | NM_ | SAP130 | CCACCCCATTCGGTTCTTCTGCCT |
| | 1700044 | | 024545.2 | | GACCTTCAAATGCCCATGTTGGCC |
| | | | | | TT |
| 62/207 | ILMN_ | mRNA | NM_ | C19orf61 | CCGGGGCTTCCACCTGACTTCCTG |
| | 1737005 | | 019108.2 | | GACTCTGAGGTCAACTTATTCCTG |
| | | | | | GT |
| 63/208 | ILMN_ | mRNA | NM_ | SGK | AGAAAGGGTTTTTATGGACCAAT |
| | 1702487 | | 005627.2 | | GCCCCAGTTGTCAGTCAGAGCCG |
| | | | | | TTGG |
| 64/209 | ILMN_ | mRNA | NM_ | AHSG | TCCTCACAGGACAGAAGCAGAGT |
| | 1730625 | | 001622.1 | | GGGTGGTGGTTATGTTTGACAGA |
| | | | | | AGGC |
| 65/210 | OA_ | miRNA | hsa-miR- | miR-21 | UAGCUUAUCAGACUGAUGUUG |
| | 000397 | | 21 | | A |
| 66/211 | ILMN_ | mRNA | NM_ | MED12 | CTTTGGTCCGGCAACTTCAACAAC |
| | 1793386 | | 005120.1 | | AGCTCTCTAATACCCAGCCACAGC |
| | | | | | CC |
| 67/212 | ILMN_ | mRNA | NM_ | CD79A | CATATACGTGTGCCGGGTCCAGG |
| | 1659227 | | 001783.3 | | AGGGCAACGAGTCATACCAGCAG |
| | | | | | TCCT |
| 68/213 | ILMN_ | mRNA | NM_ | CERK | GCTCTGATTTCCGGGGCAGCCTTT |
| | 1767475 | | 022766.4 | | CAGATGCGGCAGACATACAACAC |
| | | | | | CTG |
| 69/214 | ILMN_ | mRNA | NM_ | MRPL49 | CCCTGCCCCCAAACTGGCTAAGAC |
| | 1681324 | | 004927.2 | | AGCTTTCAGTTCCTGACTCCCCAA |
| | | | | | CT |
| 70/215 | ILMN_ | mRNA | XM_ | LOC644763 | CACTGCCGTCCCCCAAGGTCCAG |
| | 1667402 | | 927860.1 | | AATGTCAGCTCGCCTCACAAGTCA |
| | | | | | GAA |
| 71/216 | OA_ | miRNA | hsa-miR- | miR-28- | CACUAGAUUGUGAGCUCCUGGA |
| | 002446 | | 28-3p | 3p | |
| 72/217 | ILMN_ | mRNA | NM_ | ABLIM1 | GCATCCTCCTGTGTATGGAAGAG |
| | 2396672 | | 001003407.1 | | ACAGGTGACCGCTCCAGGTTGGG |
| | | | | | TGCT |
| 73/218 | ILMN_ | mRNA | NM_ | WDR37 | GAGCCGGGGCACCTTGCTGTTCG |
| | 1796464 | | 014023.3 | | CTGCTGTGTCGTCTTCTAATGTGA |
| | | | | | GCT |
| 74/219 | ILMN_ | mRNA | NM_ | SPTBN1 | AGATAGGCCAGAGCGTGGACGA |
| | 1690708 | | 003128.2 | | GGTGGAGAAGCTCATCAAGCGCC |
| | | | | | ACGAG |
| 75/220 | ILMN_ | mRNA | NM_ | ZNF274 | TCACACTGGCGCTAAGCCCTACAA |
| | 2352574 | | 016324.2 | | GTGTCAGGACTGTGGAAAAGCCT |
| | | | | | TCC |
| 76/221 | ILMN_ | mRNA | NM_ | UVRAG | CCCCTGTGGGGGCCAAAGTTTTT |
| | 1761069 | | 003369.3 | | ATGTGGGCAGATGCTGTGGTCAG |
| | | | | | GAAC |
| 77/222 | ILMN_ | mRNA | NM_ | RNF214 | CAATGGCGTGTACCCATGTATTGC |
| | 2357777 | | 2073432 | | ACAAGGAGTGTATCAAATTCTGG |
| | | | | | GCC |
| 78/223 | ILMN_ | mRNA | NM_ | YEATS2 | GCAAGTACAGAAGGAATCTATTC |
| | 1676899 | | 0180233 | | TCAGCAGGGCATAGGGCACGCAC |
| | | | | | TGGC |
| 79/224 | ILMN_ | mRNA | NM_ | PHRF1 | TCGGGTTCCTGCGCTGACACCTG |
| | 3245476 | | 020901.1 | | GTCTGTGCACCTGTGTTGCTCACA |
| | | | | | GTT |
| 80/225 | ILMN_ | mRNA | NM_ | PLAC8 | ATGCTGTCTGTGTGGAACAAGCG |
| | 2093343 | | 016619.1 | | TCGCAATGAGGACTCTCTACAGG |
| | | | | | ACCC |
| 81/226 | ILMN_ | mRNA | NM_ | TMEM66 | GAGCTCTGAAGCTTTGAATCATTC |
| | 1780141 | | 016127.4 | | AGTGGTGGAGATGGCCTTCTGGT |
| | | | | | AAC |
| 82/227 | ILMN_ | mRNA | NM_ | CETP | TGGCTCCCAACTCCTCCCTATCCT |
| | 2098013 | | 000078.1 | | AAAGGCCCACTGGCATTAAAGTG |
| | | | | | CTG |
| 83/228 | ILMN_ | mRNA | NM_ | SFRS2IP | CTGCTCCGACAGCAGCCCCAGGA |
| | 2069593 | | 0047192 | | AATACGGGAATGGTTCAGGGACC |
| | | | | | AAGT |
| 84/229 | ILMN_ | mRNA | NM_ | CTNNAL1 | CTCCTGGAAATAAACAAGCTAATT |
| | 2136446 | | 003798.1 | | CCTCTATGCCACCAGCTCCAGACA |
| | | | | | GT |
| 85/230 | ILMN_ | mRNA | NM_ | HNRNPD | GGTGACCAGCAGAGTGGTTATGG |
| | 1751368 | | 0021383 | | GAAGGTATCCAGGCGAGGTGGTC |
| | | | | | ATCA |
| 86/231 | ILMN_ | mRNA | NM_ | FAM193A | TGGGCGGGGCAGGCCTCCTTTGT |
| | 1651504 | | 0037043 | | TCTCCACAATCTACTGTCTCCGAG |
| | | | | | TGT |
| 87/232 | ILMN_ | mRNA | XM_ | FLJ36032 | GAGCTCTAACCTCTCCCCGACCCC |
| | 1691194 | | 939535.1 | | TGCAGTATCTCCCTTTGTTCAGTC |
| | | | | | TT |
| 88/233 | ILMN_ | mRNA | NM_ | MAPK7 | AGGCTTTAGCCCTGGACCCAGCA |
| | 1709623 | | 139032.1 | | GGTGAGGCTCGGCTTGGATTATT |
| | | | | | CTGC |
| 89/234 | ILMN_ | mRNA | NM_ | TCEB2 | GATGACACCTTTGAGGCCCTGTG |
| | 1733927 | | 0071082 | | CATCGAGCCGTTTTCCAGCCCGCC |
| | | | | | AGA |
| 90/235 | ILMN_ | mRNA | XR_ | LOC643332 | TGTACTGTAACCTCACAACTCCAA |
| | 3202002 | | 016287.1 | | GTCCACAGAATATTTCAAACTGCA |
| | | | | | GG |
| 91/236 | ILMN_ | mRNA | NM_ | PAFAH1B1 | GGGAGGGCAAGCTGGATTTACAG |
| | 1722276 | | 0004302 | | GTCACGGCTGGACTGAATGGGCC |
| | | | | | TTTT |
| 92/237 | ILMN_ | mRNA | NM_ | STK4 | TGAGGTCAGCAGTTTGTATGAGA |
| | 1711383 | | 006282.2 | | CATAGCTTCCTCCATTGCCCCCAC |
| | | | | | TCC |
| 93/238 | ILMN_ | mRNA | NM_ | IFI27L2 | AACATCCTCCTGGCCTCTGTTGGG |
| | 3238560 | | 032036.2 | | TCAGTGTTGGGGGCCTGCTTGGG |
| | | | | | GAA |
| 94/239 | ILMN_ | mRNA | NM_ | YWHAZ | GGCACCCTGCTTCCTTTGCTTGCA |
| | 1801928 | | 003406.2 | | TCCCACAGACTATTTCCCTCATCCT |
| | | | | | A |
| 95/240 | ILMN_ | mRNA | NM_ | UBTF | GTCCCAAAGAGTTTGATGAGGCC |
| | 1806946 | | 001076683.1 | | CTCCACACCTGCGGCCCAATCCAA |
| | | | | | GGT |
| 96/241 | ILMN_ | mRNA | NM_ | UBE2F | CCCCTGGATTGCCCCAGTCCTGTG |
| | 2164242 | | 080678.1 | | ACCATGTTGCCCTGAAGAAGACC |
| | | | | | ATC |
| 97/242 | ILMN_ | mRNA | NM_ | ASXL1 | GCTCCTGCCTCTCTCCCAACATGT |
| | 1726025 | | 015338.4 | | TTCCAGCAAGTAGATGCCCCTGTG |
| | | | | | TG |
| 98/243 | ILMN_ | mRNA | NM_ | UBFD1 | TGGCCCAGGAGACTGACCCAAAG |
| | 1700811 | | 019116.2 | | TGAAGGACATTGCCGGGAGAGG |
| | | | | | CCTGC |
| 99/244 | ILMN_ | mRNA | NM_ | SH3KBP1 | CTTTTGCTTCAGGCTAAGAGCTGC |
| | 1808501 | | 031892.1 | | CTCGCTCTTTGTCCCCCCATTAGG |
| | | | | | AT |
| 100/245 | ILMN_ | mRNA | NM_ | ZZEF1 | AGGAGGCGAAGCCCGCAGAGCA |
| | 1786396 | | 015113.3 | | AAGGTGGAAACACGTGCCTACGC |
| | | | | | TGTAA |
| 101/246 | OA_ | miRNA | hsa-miR- | miR-103 | AGCAGCAUUGUACAGGGCUAUG |
| | 000439 | | 103 | | A |
| 102/247 | ILMN_ | mRNA | NM_ | NCOA5 | AGAAGGAGGGTTTCTGGCTGTGG |
| | 1770035 | | 020967.2 | | TTCTAAATGGAGCCCCAGGAAGC |
| | | | | | TGCC |
| 103/248 | ILMN_ | mRNA | NM_ | PDE4B | GCAGTGGTGTCGTTCACCGTGAG |
| | 1782922 | | 002600.3 | | AGTCTGCATAGAACTCAGCAGTG |
| | | | | | TGCC |
| 104/249 | ILMN_ | mRNA | NM_ | RBL2 | CCCCATTCGGTGTGGTGCAGTGT |
| | 1756999 | | 005611.2 | | GAAAAGTCCTTGATTGTTCGGGT |
| | | | | | GTGC |
| 105/250 | ILMN_ | mRNA | NM_ | PHF1 | TGCCTCTGCCCAGCTCCCCATTCA |
| | 1746968 | | 024165.1 | | CACACACCGGCACTTTCATACCCT |
| | | | | | GA |
| 106/251 | ILMN_ | mRNA | NM_0011 | ACTB | CGGCTACAGCTTCACCACCACGG |
| | 1777296 | | 01.2 | | CCGAGCGGGAAATCGTGCGTGAC |
| | | | | | ATTA |
| 107/252 | ILMN_ | mRNA | NM_ | IDO2 | GCCAAGCCTTTCCCTCCCTACCTG |
| | 3237462 | | 194294.2 | | ATCACTGCTTAACGGCATGTATAA |
| | | | | | TG |
| 108/253 | OA_ | miRNA | hsa-miR- | miR-363 | AAUUGCACGGUAUCCAUCUGUA |
| | 001271 | | 363 | | |
| 109/254 | OA_00 | miRNA | hsa-miR- | miR- | UACCACAGGGUAGAACCACGG |
| | 2234 | | 140-3p | 140-3p | |
| 110/255 | ILMN_ | mRNA | NM_ | IKBKB | GTGCTGGGCCGGGGAGTCCCTGT |
| | 1727142 | | 001556.1 | | CTCTCACAGCATCTAGCAGTATTA |
| | | | | | TTA |
| 111/256 | OA_ | miRNA | hsa-miR- | miR- | GGGAGCCAGGAAGUAUUGAUG |
| | 002087 | | 505_A | 505_A | U |
| 112/257 | ILMN_ | mRNA | XM_ | LOC729273 | CATGATGGGATATCCCTGCCTAG |
| | 3240871 | | 001720501.1 | | ATCTTTCAGTGAGTCTCTACCTCA |
| | | | | | GCT |
| 113/258 | ILMN_ | mRNA | NM_ | POFUT2 | GAGAGAGGACAGTTAGGAGGGA |
| | 2376667 | | 133635.4 | | CAGACAGCTCTTCCTTTCGGAGCC |
| | | | | | TGGC |
| 114/259 | ILMN_ | mRNA | NM_ | TNFAIP1 | CAGTGTCTCAGTCTTTTTTGCCGA |
| | 1655429 | | 021137.3 | | GAAAGCACAGTAGTCTGGGACTG |
| | | | | | GGC |
| 115/260 | ILMN_ | mRNA | NM_ | CYP4F3 | CAGCTCGGAGGAAGGTCTCCTAT |
| | 2089484 | | 000896.2 | | ACACACAAAGCCTGGCATGCACC |
| | | | | | TTCG |
| 116/261 | ILMN_ | mRNA | NM_ | IL13RA2 | GTAACCGGTCTGCTTTTGCGTAAG |
| | 1688722 | | 000640.2 | | CCAAACACCTACCCAAAAATGATT |
| | | | | | CC |
| 117/262 | ILMN_ | mRNA | NM_ | DYRK1A | TGACTGGTCTCCTAACCAAGGTGC |
| | 1660663 | | 130437.2 | | ACTGAGAAGCAATCAACGGGTCG |
| | | | | | GTC |
| 118/263 | ILMN_ | mRNA | NM_ | ARCN1 | GCTGGTTGAAAAGTACCACTCCC |
| | 1699703 | | 001655.3 | | ACTCTGAACATCTGGCCGTCCCTG |
| | | | | | CAA |
| 119/264 | ILMN_ | mRNA | NM_ | RERE | GCCCTGACCTTCATGGTGTCTTTG |
| | 1802380 | | 001042682.1 | | AAGCCCAACCACTCGGTTTCCTTC |
| | | | | | GG |
| 120/265 | ILMN_ | mRNA | NM_ | LRRTM4 | AGGAGAGAGGTTTGAGTTCTGGG |
| | 1685472 | | 024993.3 | | TATCCTCCCTTTCTGTAACAGCCTC |
| | | | | | AA |
| 121/266 | ILMN_ | mRNA | NM_ | IFI27L2 | CCCAGCTGAACCCGAGGCTAAAG |
| | 1740319 | | 032036.2 | | AAGATGAGGCAAGAGAAAATGT |
| | | | | | ACCCC |
| 122/267 | ILMN_ | mRNA | NM_ | OR8D1 | CACCTTGGTGCCCACCCTAGCTGT |
| | 1742753 | | 001002917.1 | | TGCTGTCTCCTATGCCTTCATCCTC |
| | | | | | T |
| 123/268 | ILMN_ | mRNA | XR_ | LOC728533 | CTATACTCCTTTGGCCCATAGCTA |
| | 3226045 | | 015610.2 | | AGGTCATCCTTCCCCACAGGGGT |
| | | | | | GGC |
| 124/269 | ILMN_ | mRNA | NM_ | CCDC90A | GAGAACAGAAATAGTGGCATTGC |
| | 1761961 | | 001031713.2 | | ATGCCCAGCAAGATCGGGCCCTT |
| | | | | | ACCC |
| 125/270 | ILMN_ | mRNA | NM_ | WBP11 | GCTAACATCCATTCCCTTTCATACC |
| | 1766435 | | 016312.2 | | ACCATTTTCACCCTGTTTCTTCCCC |
| 126/271 | ILMN_ | mRNA | NM_ | KIAA1920 | CATCTGGACCCCTCCCCCTCTATC |
| | 1792432 | | 052919.1 | | CCTAACCCTGTCTAAACTAATGGC |
| | | | | | GC |
| 127/272 | ILMN_ | mRNA | NM_ | MAEA | TCCGCCCATGATGCTGCCCAACG |
| | 2327090 | | 005882.3 | | GCTACGTCTACGGCTACAATTCTC |
| | | | | | TGC |
| 128/273 | ILMN_ | mRNA | NM_ | EIF4G2 | CTCTTATCCCAGCTGCAAGGACAG |
| | 2279635 | | 001418.3 | | TCGAAGGATATGCCACCTCGGTTT |
| | | | | | TC |
| 129/274 | ILMN_ | mRNA | NM_ | PDE7A | TGGAAGGGACTGCAGAGAGAAC |
| | 2278819 | | 002603.1 | | AGTCGAGCAGTGAGGACACTGAT |
| | | | | | GCTGC |
| 130/275 | ILMN_ | mRNA | NM_ | KBTBD4 | GCCTGTTCTCTGCCATTCCCTAGT |
| | 1687092 | | 018095.3 | | CATCCTGTGCCTCACCACAGCTTG |
| | | | | | CT |
| 131/276 | ILMN_ | mRNA | NM_ | PRDX4 | CTGCCCTGCTGGCTGGAAACCTG |
| | 2222234 | | 006406.1 | | GTAGTGAAACAATAATCCCAGAT |
| | | | | | CCAG |
| 132/277 | ILMN_ | mRNA | NM_ | ARHGAP25 | GACCACGTCCAGTGAAGACATTT |
| | 1777998 | | 014882.2 | | GAGGCAGCACATCTCAGGACCCA |
| | | | | | GGCA |
| 133/278 | ILMN_ | mRNA | NM_ | ZBED4 | GCATCTCCACGCTCTGAAGCTGTC |
| | 1782129 | | 014838.2 | | TTTCAAAATGTGTGCACTGACCCC |
| | | | | | CT |
| 134/279 | ILMN_ | mRNA | NM_ | XPC | AGTCTTCATCTGTCCGACAAGTTC |
| | 1790807 | | 004628.3 | | ACTCGCCTCGGTTGCGGACCTAG |
| | | | | | GAC |
| 135/280 | ILMN_ | mRNA | NM_ | POMP | GATCCATCACAAAGCGAAGTCAT |
| | 1693287 | | 301592.3 | | GGGAGAGCCACACTTGATGGTGG |
| | | | | | AATA |
| 136/281 | ILMN_ | mRNA | NM_ | PLOD2 | ACAAAGTTGTTGAGCCTTGCTTCT |
| | 1771599 | | 000935.2 | | TCCGTTTTGCCCTTTGTCTCGCTCC |
| | | | | | T |
| 137/282 | ILMN_ | mRNA | NM_ | RBM15B | CTGCCCCAGCTACAGAGACGGCC |
| | 1673024 | | 013286.3 | | GAAATGCTTTCACTCCTTAGCTTT |
| | | | | | GCC |
| 138/283 | ILMN_ | mRNA | NM_ | MAT2B | GGAGAAAGAGCTCTCTATACACT |
| | 1680246 | | 013283.3 | | TTGTTCCCGGGAGCTGTCGGCTG |
| | | | | | GTGG |
| 139/284 | ILMN_ | mRNA | NM_ | PIK3CD | AGCTCTGTTCTGATTCACCAGGG |
| | 1766275 | | 005026.2 | | GTCCGTCAGTAGTCATTGCCACCC |
| | | | | | GCG |
| 140/285 | ILMN_ | mRNA | NM_ | STX8 | GCCAGAGGAGACCAGAGGCTTG |
| | 1752895 | | 004853.1 | | GGTTTTGATGAAATCCGGCAACA |
| | | | | | GCAGC |
| 141/286 | ILMN_ | mRNA | NM_ | SFRS4 | TGGCCTTTCCTACAGGGAGCTCA |
| | 2175075 | | 005626.3 | | GTAACCTGGACGGCTCTAAGGCT |
| | | | | | GGAA |
| 142/287 | ILMN_ | mRNA | NM_ | CORO1A | GATGCTGGGCCCCTCCTCATCTCC |
| | 1713749 | | 007074.2 | | CTCAAGGATGGCTACGTACCCCC |
| | | | | | AAA |
| 143/288 | ILMN_ | mRNA | NM_ | FKSG30 | CCTGGGCATGGAATCCTGTGGCA |
| | 1814998 | | 001017421.1 | | TCCACAAAACTACCTTCAACTCCA |
| | | | | | TAG |
| 144/289 | ILMN_ | mRNA | NM_ | DDX24 | AAGAAGCCGAAGGAGCCACAGC |
| | 1700628 | | 020414.3 | | CGGAACAGCCACAGCCAAGTACA |
| | | | | | AGTGC |
| 145/290 | ILMN_ | mRNA | NM_ | AHSA1 | CCACCATCACCTTGACCTTCATCG |
| | 1703617 | | 012111.1 | | ACAAGAACGGAGAGACTGAGCT |
| | | | | | GTGC |
| 146/291 | ILMN_ | mRNA | NM_ | PIK3C2B | CCATAACTGGAGAAAGAAGCTCC |
| | 2117323 | | 002646.2 | | ATTGACCGAAGCCACAGGGCAGC |
| | | | | | ATGG |
| 147/292 | ILMN_ | mRNA | NM_ | ZNF134 | ACCTGAGGCCCTTAACCTTTCTCT |
| | 1768809 | | 003435.2 | | CAGTGCTCGCCTTCCCCCAGAATC |
| | | | | | CC |

Table 3 identifies the 18 genes and 5 miRNAs that overlap between the mRNA and miRNA sets of Tables 1 and 2.

TABLE 3

| ID | TYPE | ACCESSION | SYMBOL |
|---|---|---|---|
| OA_002285 | miRNA | hsa-miR-186 | miR-186 |
| OA_000442 | miRNA | hsa-miR-106b | miR-106b |
| ILMN_2382758 | mRNA | NM_134442.2 | CREB1 |
| ILMN_1805996 | mRNA | NM_015477.1 | SIN3A |
| ILMN_3179371 | mRNA | NM_031263.2 | HNRNPK |
| ILMN_2227573 | mRNA | NM_004832.1 | GSTO1 |
| OA_000397 | miRNA | hsa-miR-21 | miR-21 |
| ILMN_3274914 | mRNA | XR_038906.1 | LOC648927 |
| ILMN_1700044 | mRNA | NM_024545.2 | SAP130 |
| ILMN_1806946 | mRNA | NM_001076683.1 | UBTF |
| ILMN_1770035 | mRNA | NM_020967.2 | NCOA5 |
| OA_002234 | miRNA | hsa-miR-140-3p | miR-140-3p |
| ILMN_1730999 | mRNA | NM_003292.2 | TPR |
| ILMN_1751368 | mRNA | NM_002138.3 | HNRNPD |
| ILMN_1766435 | mRNA | NM_016312.2 | WBP11 |
| ILMN_2054121 | mRNA | NM_207409.1 | C6orf126 |
| ILMN_1793386 | mRNA | NM_005120.1 | MED12 |
| ILMN_1790807 | mRNA | NM_004628.3 | XPC |
| ILMN_1681324 | mRNA | NM_004927.2 | MRPL49 |
| OA_001271 | miRNA | hsa-miR-363 | miR-363 |
| ILMN_1685472 | mRNA | NM_024993.3 | LRRTM4 |
| ILMN_1688722 | mRNA | NM_000640.2 | IL13RA2 |
| ILMN_1740319 | mRNA | NM_032036.2 | IFI27L2 |

The genes and miRNA identified in Tables 1-3 are publically available. One skilled in the art may readily reproduce these compositions or probe and primer sequences that hybridize thereto by use of the sequences of the mRNA and miRNA. All such sequences are publically available from conventional sources, such as Illumina, ABI OpenArray, GenBank or NCBI databases. The website identified as www.mirbase.org is also another public source for such sequences.

In the context of the compositions and methods described herein, reference to “at least two,” “at least five,” etc. of the combined mRNA and miRNAs listed in any particular combined set means any and all combinations of the mRNAs and miRNAs identified. Specific mRNA and miRNAs for the disease profile do not have to be in rank order as in Tables 1 and 2 and may be any combination of mRNA and miRNA identified herein, and/or in Table 3.

The term “polynucleotide,” when used in singular or plural form, generally refers to any polyribonucleotide or polydeoxribonucleotide, which may be unmodified RNA or DNA or modified RNA or DNA. Thus, for instance, polynucleotides as defined herein include, without limitation, single- and double-stranded DNA, DNA including single- and double-stranded regions, single- and double-stranded RNA, and RNA including single- and double-stranded regions, hybrid molecules comprising DNA and RNA that may be single-stranded or, more typically, double-stranded or include single- and double-stranded regions. In addition, the term “polynucleotide” as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide. The term “polynucleotide” specifically includes cDNAs. The term includes DNAs (including cDNAs) and RNAs that contain one or more modified bases. Thus, DNAs or RNAs with backbones modified for stability or for other reasons are “polynucleotides” as that term is intended herein. Moreover, DNAs or RNAs comprising unusual bases, such as inosine, or modified bases, such as tritiated bases, are included within the term “polynucleotides” as defined herein. In general, the term “polynucleotide” embraces all chemically, enzymatically and/or metabolically modified forms of unmodified polynucleotides, as well as the chemical forms of DNA and RNA characteristic of viruses and cells, including simple and complex cells.

The term “oligonucleotide” refers to a relatively short polynucleotide, including, without limitation, single-stranded deoxyribonucleotides, single- or double-stranded ribonucleotides, RNA:DNA hybrids and double-stranded DNAs. Oligonucleotides, such as single-stranded DNA probe oligonucleotides, are often synthesized by chemical methods, for example using automated oligonucleotide synthesizers that are commercially available. However, oligonucleotides can be made by a variety of other methods, including in vitro recombinant DNA-mediated techniques and by expression of DNAs in cells and organisms.

As used herein, the term “antibody” refers to an intact immunoglobulin having two light and two heavy chains or any fragments thereof. Thus a single isolated antibody or fragment may be a polyclonal antibody, a high affinity polyclonal antibody, a monoclonal antibody, a synthetic antibody, a recombinant antibody, a chimeric antibody, a humanized antibody, or a human antibody. The term “antibody fragment” refers to less than an intact antibody structure, including, without limitation, an isolated single antibody chain, a single chain Fv construct, a Fab construct, a light chain variable or complementarity determining region (CDR) sequence, etc.

The terms “differentially expressed gene transcript or mRNA” or “differentially expressed miRNA”, “differential expression” and their synonyms, which are used interchangeably, refer to a gene or miRNA sequence whose expression is activated to a higher or lower level in a subject suffering from a disease, specifically cancer, such as lung cancer, relative to its expression in a control subject. The terms also include genes or miRNA whose expression is activated to a higher or lower level at different stages of the same disease. It is also understood that a differentially expressed gene or miRNA may be either activated or inhibited at the nucleic acid level or protein level, or may be subject to alternative splicing to result in a different polypeptide product. Such differences may be evidenced by a change in mRNA levels, surface expression, secretion or other partitioning of a polypeptide, for example. Differential gene expression may include a comparison of expression between two or more genes or their gene products, or a comparison of the ratios of the expression between two or more genes or their gene products, or even a comparison of two differently processed products of the same gene, which differ between normal subjects, non-health controls and subjects suffering from a disease, specifically cancer, or between various stages of the same disease. Differential expression includes both quantitative, as well as qualitative, differences in the temporal or cellular expression pattern in a gene or its expression products among, for example, normal and diseased cells, or among cells which have undergone different disease events or disease stages. For the purpose of this invention, “differential gene expression” is considered to be present when there is a statistically significant (p<0.05) difference in gene expression between the subject and control samples.

The term “over-expression” with regard to an RNA transcript is used to refer to the level of the transcript determined by normalization to the level of reference mRNAs, which might be all measured transcripts in the specimen or a particular reference set of mRNAs.

The phrase “amplification” refers to a process by which multiple copies of a gene or gene fragment or miRNA are formed in a particular cell or cell line. The duplicated region (a stretch of amplified DNA) is often referred to as “amplicon.” Usually, the amount of the messenger RNA (mRNA) produced, i.e., the level of gene expression, also increases in the proportion of the number of copies made of the particular gene expressed.

The term “prognosis” is used herein to refer to the prediction of the likelihood of cancer-attributable death or progression, including recurrence, metastatic spread, and drug resistance, of a neoplastic disease, such as lung cancer. The term “prediction” is used herein to refer to the likelihood that a patient will respond either favorably or unfavorably to a drug or set of drugs, and also the extent of those responses, or that a patient will survive, following surgical removal of the primary tumor and/or chemotherapy for a certain period of time without cancer recurrence. The predictive methods of the present invention can be used clinically to make treatment decisions by choosing the most appropriate treatment modalities for any particular patient. The predictive methods described herein are valuable tools in predicting if a patient is likely to respond favorably to a treatment regimen, such as surgical intervention, chemotherapy with a given drug or drug combination, and/or radiation therapy, or whether long-term survival of the patient, following surgery and/or termination of chemotherapy or other treatment modalities is likely.

The term “long-term” survival is used herein to refer to survival for at least 1 year, more preferably for at least 3 years, most preferably for at least 7 years following surgery or other treatment.

Stringency” of hybridization reactions is readily determinable by one of ordinary skill in the art, and generally is an empirical calculation dependent upon probe length, washing temperature, and salt concentration. In general, longer probes require higher temperatures for proper annealing, while shorter probes need lower temperatures. Hybridization generally depends on the ability of denatured DNA to re-anneal when complementary strands are present in an environment below their melting temperature. The higher the degree of desired homology between the probe and hybridizable sequence, the higher is the relative temperature which can be used. As a result, it follows that higher relative temperatures would tend to make the reaction conditions more stringent, while lower temperatures less so. Various published texts provide additional details and explanation of stringency of hybridization reactions.

In the context of the compositions and methods described herein, reference to “three or more,” “at least five,” etc. of the mRNA and miRNA listed in any particular gene set (e.g., Table 1, 2 or 3) means any one or any and all combinations of the mRNA and miRNA listed. For example, suitable combined mRNA and miRNA expression profiles include profiles containing any number between at least 3 through 145 mRNA and miRNA from Table 1, 2 and/or 3. In one embodiment, expression profiles formed by mRNA and miRNA selected from the table are preferably used in rank order, e.g., genes ranked in the top of the list demonstrated more significant discriminatory results in the tests, and thus may be more significant in a profile than lower ranked genes. However, in other embodiments the genes forming a useful gene profile do not have to be in rank order and may be any gene from the respective table.

It should be understood that while various embodiments in the specification are presented using “comprising” language, under various circumstances, a related embodiment is also be described using “consisting of” or “consisting essentially of” language. It is to be noted that the term “a” or “an”, refers to one or more, for example, “an miRNA,” is understood to represent one or more miRNAs. As such, the terms “a” (or “an”), “one or more,” and “at least one” are used interchangeably herein.

Unless defined otherwise in this specification, technical and scientific terms used herein have the same meaning as commonly understood by one of ordinary skill in the art to which this invention belongs and by reference to published texts, which provide one skilled in the art with a general guide to many of the terms used in the present application.

The mRNA and miRNA lung cancer and lung disease signatures or gene and miRNA expression profiles identified herein and through use of the gene collections of Table 1, 2 and/or 3 may be further optimized to reduce or increase the numbers of genes and miRNA and thereby increase accuracy of diagnosis.

Gene (mBNA) Expression Profiling Methods

Methods of gene (mRNA) expression profiling that were used in generating the profiles useful in the compositions and methods described herein or in performing the diagnostic steps using the compositions described herein are known and well summarized in U.S. Pat. No. 7,081,340 and in International Patent Application Publication No. WO2010/054233, incorporated by reference herein. Such methods of gene expression profiling include methods based on hybridization analysis of polynucleotides, methods based on sequencing of polynucleotides, and proteomics-based methods. The most commonly used methods known in the art for the quantification of mRNA expression in a sample include northern blotting and in situ hybridization; RNAse protection assays; and PCR-based methods, such as RT-PCR. Alternatively, antibodies may be employed that can recognize specific duplexes, including DNA duplexes, RNA duplexes, and DNA-RNA hybrid duplexes or DNA-protein duplexes. Representative methods for sequencing-based gene expression analysis include Serial Analysis of Gene Expression (SAGE), and gene expression analysis by massively parallel signature sequencing (MPSS).

Briefly described, the most sensitive and most flexible quantitative method is RT-PCR, which can be used to compare mRNA levels in different sample populations, in normal and tumor tissues, with or without drug treatment, to characterize patterns of gene expression, to discriminate between closely related mRNAs, and to analyze RNA structure. The first step is the isolation of mRNA from a target sample (e.g., typically total RNA isolated from human PBMC in this case). mRNA can be extracted, for example, from frozen or archived paraffin-embedded and fixed (e.g. formalin-fixed) tissue samples. RNA isolation can be performed using purification kit, buffer set and protease from commercial manufacturers, according to the manufacturer's instructions. Exemplary commercial products include TRI-REAGENT, Qiagen RNeasy mini-columns, MASTERPURE Complete DNA and RNA Purification Kit (EPICENTRE®, Madison, Wis.), Paraffin Block RNA Isolation Kit (Ambion, Inc.) and RNA Stat-60 (Tel-Test). Conventional techniques such as cesium chloride density gradient centrifugation may also be employed.

The first step in gene expression profiling by RT-PCR is the reverse transcription of the RNA template into cDNA, followed by its exponential amplification in a PCR reaction. The reverse transcription step is typically primed using specific primers, random hexamers, or oligo-dT primers, depending on the circumstances and the goal of expression profiling. See, e.g., manufacturer's instructions accompanying the product GENEAMP RNA PCR kit (Perkin Elmer, Calif., USA). The derived cDNA can then be used as a template in the subsequent RT-PCR reaction.

The PCR step generally uses a thermostable DNA-dependent DNA polymerase, such as the Taq DNA polymerase, which has a 5′-3′ nuclease activity but lacks a 3′-5′ proofreading endonuclease activity, e.g., TAQMAN® PCR. The selected polymerase hydrolyzes a hybridization probe bound to its target amplicon and two oligonucleotide primers generate an amplicon. The third oligonucleotide, or probe, preferably labeled, is designed to detect nucleotide sequence located between the two PCR primers. TaqMan® RT-PCR can be performed using commercially available equipment.

Real time PCR is comparable both with quantitative competitive PCR, where internal competitor for each target sequence is used for normalization, and with quantitative comparative PCR using a normalization gene contained within the sample, or a housekeeping gene for RT-PCR. Another PCR method is the MassARRAY-based gene expression profiling method (Sequenom, Inc., San Diego, Calif.). Still other embodiments of PCR-based techniques which are known to the art and may be used for gene expression profiling include, e.g., differential display, amplified fragment length polymorphism (iAFLP), and BeadArray™ technology (Illumina, San Diego, Calif.) using the commercially available Luminex100 LabMAP system and multiple color-coded microspheres (Luminex Corp., Austin, Tex.) in a rapid assay for gene expression; and high coverage expression profiling (HiCEP) analysis.

RNA expression profiles are obtained from the blood of subjects by centrifugation using a CPT tube, a Ficoll gradient or equivalent density separation to remove red cells and granulocytes and subsequent extraction of the RNA using TRIZOL tri-reagent, RNALATER reagent or a similar reagent to obtain RNA of high integrity. The amount of individual messenger RNA species was determined using microarrays and/or Quantitative polymerase chain reaction.

Among the other procedures employed in obtaining the RNA expression levels for profiles are RT-PCR with analytic use of machine-learning algorithms, such as SVM with Recursive Feature Elimination (SVM-RFE) or other classification algorithm such as Penalized Discriminant Analysis (PDA) (see International Patent Application Publication No WO 2004/105573, published Dec. 9, 2004) to obtain a mathematical function whose coefficients act on the input RNA gene express values and output a “SCORE” whose value determines the class of the individual and the confidence of the prediction. Having determined this function by analysis of numerous subjects known to be of the classes whose members are to be subsequently distinguished, it is used to classify subjects for their disease states.

Differential gene expression can also be identified, or confirmed using the microarray technique, also described in detail in International Patent Application Publication No. WO2010/054233. Thus, the expression profile of lung cancer/lung disease-associated genes can be measured in either fresh or paraffin-embedded tissue, using microarray technology. In this method, polynucleotide sequences of interest (including cDNAs and oligonucleotides) are plated, or arrayed, on a microchip or glass substrate. The arrayed sequences are then hybridized with specific DNA probes from cells or tissues of interest. The microarrayed genes, immobilized on the microchip are suitable for hybridization under stringent conditions. Labeled cDNA probes applied to the chip hybridize with specificity to each spot of DNA on the array. After stringent washing to remove non-specifically bound probes, the chip is scanned by confocal laser microscopy or by another detection method, such as a CCD camera. Quantitation of hybridization of each arrayed element allows for assessment of corresponding mRNA abundance. Microarray analysis can be performed by commercially available equipment, following manufacturer's protocols.

Other useful methods summarized by U.S. Pat. No. 7,081,340, and incorporated by reference herein include Serial Analysis of Gene Expression (SAGE) and Massively Parallel Signature Sequencing (MPSS).

Immunohistochemistry methods and proteomic methods are also suitable for detecting the expression levels of the gene expression products of the genes described for use in the methods and compositions herein and are valuable supplements to other methods of gene expression profiling, and can be used, alone or in combination with other methods, to detect the gene expression products of the combined gene and miRNA profiles described herein. Antibodies or antisera, preferably polyclonal antisera, and most preferably monoclonal antibodies, or other protein-binding ligands specific for each marker are used to detect expression. The antibodies can be detected by direct labeling of the antibodies themselves, for example, with radioactive labels, fluorescent labels, hapten labels such as, biotin, or an enzyme such as horse radish peroxidase or alkaline phosphatase. Alternatively, unlabeled primary antibody is used in conjunction with a labeled secondary antibody, comprising antisera, polyclonal antisera or a monoclonal antibody specific for the primary antibody. Protocols and kits for immunohistochemical analyses are well known in the art and are commercially available.

In performing assays and methods of this invention, these same techniques can be used to obtain the mRNA express level components for the combined mRNA and miRNA profiles, and the patient's profile compared with the appropriate reference profile, and diagnosis or treatment recommendation selected based on this information.

Methods of Detecting/Quantifying miRNA

Methods that may be employed in obtaining, detecting and quantifying miRNA expression are known and may be used to accomplish the diagnostic goals of the present invention. See, for example, the techniques described in the examples below, as well as in e.g., International Patent Application Publication No. WO2008/073923; US Published Patent Application No. 2006/0134639, U.S. Pat. Nos. 6,040,138 and 8,476,420, among others.

For example, the biological samples may be collected using the proprietary PaxGene Blood RNA System (PreAnalytiX, a Qiagen, BD company). The PAXgene Blood RNA System comprises two integrated components: PAXgene Blood RNA Tube and the PAXgene Blood RNA Kit. Blood samples are drawn directly into PAXgene Blood RNA Tubes via standard phlebotomy technique. These tubes contain a proprietary reagent that immediately stabilizes intracellular RNA, minimizing the ex-vivo degradation or up-regulation of RNA transcripts. The ability to eliminate freezing, batch samples, and to minimize the urgency to process samples following collection, greatly enhances lab efficiency and reduces costs.

Thereafter, the miRNA are detected and/or measured using a variety of assays. The most sensitive and most flexible quantitative method is real-time polymerase chain reaction (RT-PCR), which can be used to compare miRNA levels in different sample populations, in normal and tumor tissues, with or without drug treatment, to characterize patterns of miRNA expression, to discriminate between closely related miRNAs, and to analyze RNA structure. This method can be employed by using conventional RT-PCR assay kits according to manufacturers' instructions, such as TaqMan® RT-PCR (Applied Biosystems).

The first step is the isolation of RNA from a target sample (e.g., typically total RNA isolated from human whole blood in this case). General methods for mRNA extraction are well known in the art, e.g., in standard textbooks of molecular biology. RNA isolation can be performed using a purification kit, buffer set and protease from commercial manufacturers, according to the manufacturer's instructions. Exemplary commercial products include TRI-REAGENT, Siegen RNeasy mini-columns, MASTERPURE Complete DNA and RNA Purification Kit (EPICENTRE®, Madison, Wis.) and others. Conventional techniques such as cesium chloride density gradient centrifugation may also be employed.

In the reverse transcription step, cDNA is reverse transcribed from mRNA samples using primers specific for the miRNAs to be detected. Methods for reverse transcription are well known in the art, e.g., in standard textbooks of molecular biology. Briefly, RNA is first incubated with a primer at 70° C. to denature RNA secondary structure and then quickly chilled on ice to let the primer anneal to the RNA. Other components are added to the reaction including dNTPs, RNase inhibitor, reverse transcriptase and reverse transcription buffer. The reverse transcription reaction is extended at 42° C. for 1 hr. The reaction is then heated at 70° C. to inactivate the enzyme.

In the RT-PCR step, PCR products are amplified from the cDNA samples. PCR product accumulation is measured through a dual-labeled fluorigenic probe (i.e., TAQMAN® probe). Real time PCR is compatible both with quantitative competitive PCR, where an internal competitor for each target sequence is used for normalization, and with quantitative comparative PCR using a normalization miRNA contained within the sample, or a housekeeping miRNA for RT-PCR. For further details see, e.g. Held et al., Genome Research 6:986 994 (1996). TaqMan® RT-PCR can be performed using commercially available equipment. To minimize errors and the effect of sample-to-sample variation, RT-PCR is usually performed using an internal standard. The ideal internal standard is expressed as a constant level among different tissues, and is unaffected by the experimental treatment. RNAs most frequently used to normalize patterns of miRNA expression are mRNAs for the housekeeping miRNAs glyceraldehydes-3phospate-dehydrogenase (GAPDH) and (3-actin.

The steps of a representative protocol from profiling miRNA expression using fixed, paraffin-embedded tissues as the RNA source, including mRNA isolation, purification, primer extension and amplification are known to those of skill in the art. Briefly, a representative process starts with cutting about 10 μm thick sections of paraffin-embedded tumor tissue samples. The RNA is then extracted, and protein and DNA are removed. After analysis of the RNA concentration, RNA repair and/or amplification steps may be included, if necessary, and RNA is reverse transcribed using miRNA specific promoters followed by RT-PCR.

The specific techniques identified in the examples below demonstrate the state of the art. However, other conventional methods of miRNA isolation, detection and quantification can be employed in these methods. Still other methods of detecting and/or measuring miRNA may be employed, using antibodies or fragments thereof. A recombinant molecule bearing a sequence that binds to the miRNA may also be used in these methods. It should be understood that any antibody, antibody fragment, or mixture thereof that binds a specified miRNA as defined herein may be employed in the methods to obtain the miRNA expression levels for the combined mRNA and miRNA profile, regardless of how the antibody or mixture of antibodies was generated.

Similarly, methods using genomic or other hybridization probes to identify the miRNA sequences are useful herein. In another embodiment, a suitable assay detection assay is an immunohistochemical assay, a hybridization assay, a counter immuno-electrophoresis, a radioimmunoassay, radioimmunoprecipitation assay, a dot blot assay, an inhibition of competition assay, or a sandwich assay.

Any of the methods described above or otherwise herein may be performed by a computer processor or computer-programmed instrument that generates numerical or graphical data useful in the diagnosis or detection of the condition or differentiation between two conditions.

### Compositions

The methods for diagnosing lung cancer and lung disease utilizing defined combined gene (mRNA) and miRNA expression profiles permits the development of simplified diagnostic tools for diagnosing lung cancer, e.g., NSCLC or diagnosing a specific stage (early, stage I, stage II or late) of lung cancer, diagnosing a specific type of lung cancer (e.g., AC vs. LSCC), diagnosing a type of lung disease, e.g., COPD or benign lung nodules, or monitoring the effect of therapeutic or surgical intervention for determination of further treatment or evaluation of the likelihood of recurrence of the cancer or disease.

Thus, a composition for such diagnosis or evaluation in a mammalian subject as described herein can be a kit or a reagent. For example, one embodiment of a composition includes a substrate upon which the ligands used to detect and quantitate mRNA and miRNA are immobilized. The reagent, in one embodiment, is an amplification nucleic acid primer (such as an RNA primer) or primer pair that amplifies and detects a nucleic acid sequence of the mRNA or miRNA. In another embodiment, the reagent is a polynucleotide probe that hybridizes to the target sequence. In another embodiment, the reagent is an antibody or fragment of an antibody. The reagent can include multiple said primers, probes or antibodies, each specific for at least one mRNA and miRNA of Table 1, 2 or 3. Optionally, the reagent can be associated with a conventional detectable label. As used herein, “labels” or “reporter molecules” are chemical or biochemical moieties useful for labeling a nucleic acid (including a single nucleotide), polynucleotide, oligonucleotide, or protein ligand, e.g., amino acid or antibody. “Labels” and “reporter molecules” include fluorescent agents, chemiluminescent agents, chromogenic agents, quenching agents, radionucleotides, enzymes, substrates, cofactors, inhibitors, magnetic particles, and other moieties known in the art. “Labels” or “reporter molecules” are capable of generating a measurable signal and may be covalently or noncovalently joined to an oligonucleotide or nucleotide (e.g., a non-natural nucleotide) or ligand.

In another embodiment, the composition is a kit containing the relevant multiple polynucleotides or oligonucleotide probes or ligands, optional detectable labels for same, immobilization substrates, optional substrates for enzymatic labels, as well as other laboratory items. In still another embodiment, at least one polynucleotide or oligonucleotide or ligand is associated with a detectable label. In certain embodiments, the reagent is immobilized on a substrate. Exemplary substrates include a microarray, chip, microfluidics card, or chamber.

Such a composition contains in one embodiment more than one polynucleotide or oligonucleotide, wherein each polynucleotide or oligonucleotide hybridizes to a different gene or a different miRNA from a mammalian biological sample, e.g., blood, serum, or plasma. The mRNA and miRNA, in one embodiment, are selected from those listed in Table 1, 2 and/or 3. Table 1 contains one embodiment of the approximately top 145 genes and miRNA identified by the inventors as representative of a profile or signature indicative of the presence of a lung cancer. This collection of genes and miRNA is those for which the mRNA and miRNA expression is altered (i.e., increased or decreased) versus the same mRNA and miRNA expression in the biological sample of a reference control. Table 2 contains one embodiment of the approximately top 147 genes and miRNA identified by the inventors as representative of another profile or signature indicative of the presence of a lung cancer. This collection of genes and miRNA is those for which the mRNA and miRNA expression is altered (i.e., increased or decreased) versus the same mRNA and miRNA expression in the biological sample of a reference control. Table 3 contains those mRNA and miRNA that overlap between Tables 1 and 2.

In one embodiment, the targeted mRNA and miRNA are selected from those ranked 1 to 119 in Table 1. In another embodiment, ligands to mRNA and miRNA in addition to those targets ranked in Table 1 are included in a composition of this invention. In one embodiment, the composition contains ligands targeting a single mRNA of Table 1 and ligands targeting a single miRNA of Table 1. In another embodiment, the composition contains more than one ligand that targets the same mRNA or the same miRNA.

In one embodiment, the targeted mRNA and miRNA are selected from all targets identified in Table 1. In another embodiment, the targeted mRNA and miRNA are selected from some or all targets identified in Table 2. In another embodiment, ligands to mRNA and miRNA in addition to those targets ranked in Table 1 and 2 are included in a composition of this invention. In one embodiment, the composition contains ligands targeting a single mRNA of Table 1 or 2 and ligands targeting a single miRNA of Table 1 or 2. In another embodiment, the composition contains more than one ligand that targets the same mRNA or the same miRNA, i.e., at least 5, 10, 20, 50, 75, 100, 130, 140 or more of the combinations of those Tables.

In another embodiment, a composition for diagnosing lung cancer in a mammalian subject includes three or more PCR primer-probe sets. Each primer-probe set amplifies a different polynucleotide sequence from two or more mRNA found in the biological sample of the subject coupled with a primer or probe or set amplifying a different polynucleotide sequence from one or more miRNA found in the biological sample of the subject. In another embodiment, a composition for diagnosing lung cancer in a mammalian subject includes three or more PCR primer-probe sets. Each primer-probe set amplifies a different polynucleotide sequence from one or more mRNA found in the biological sample of the subject coupled with a primer or probe or set amplifying a different polynucleotide sequence from two or more miRNA found in the biological sample of the subject.

Still other embodiments include PCR primers, probes or sets sufficient to amplify all of the ranked mRNA and miRNA of 1-119 or all mRNA and miRNA targets of Table 1, 119 or all mRNA and miRNA targets of Table 2, and/or all mRNA and miRNA targets of Table 3. Thus, in another embodiment, ligands are generated to at least mRNA and miRNA from Table 1, 2 or 3 for use in the composition. In still another embodiment, PCR primers and probes are generated to at least 25 mRNA and miRNA from Table 1, 2 and/or 3 for use in the composition. In still another embodiment, PCR primers and probes are generated to at least 50 mRNA and miRNA from Table 1, 2 and/or 3 for use in the composition. In still another embodiment, PCR primers and probes are generated to at least 75 mRNA and miRNA from Table 1, 2 and/or 3 for use in the composition. In still another embodiment, PCR primers and probes are generated to at least 100 mRNA and miRNA from Table 1 or Table 2 for use in the composition. In still another embodiment, PCR primers and probes are generated to at least 125 mRNA and miRNA from Table 1 or 2 for use in the composition. One of skill in the art will recognize that all integers occurring between the numbers specified above are included in this disclosure, even if not specifically recited herein. The selected genes and miRNA from Table 1, 2 or 3 need not be in rank order; rather any combination that clearly shows a difference in expression between the reference control to the diseased patient is useful in such a composition.

Still other embodiments include PCR primers, probes or sets sufficient to amplify smaller subsets of the ranked mRNA and miRNA targets of Table 1. Still other embodiments include PCR primers, probes or sets sufficient to amplify smaller subsets of the ranked mRNA and miRNA targets of Table 1 with PCR primers, probes or sets sufficient to amplify other mRNA and miRNA targets found to be changed characteristically in a lung disease or cancer.

These selected genes and miRNA form a combined gene/miRNA expression profile or signature which is distinguishable between a subject having lung cancer or another lung disease and a selected reference control. In one embodiment, significant changes in the combined mRNA and miRNA expression in the patient's biological sample, e.g., blood, from that of the reference correlate with a diagnosis of lung cancer, e.g., non-small cell lung cancer (NSCLC). In one embodiment, significant changes in the combined mRNA and miRNA expression in the patient's biological sample, e.g., blood, from that of the reference correlate with a diagnosis of a stage of such cancer. In one embodiment, significant changes in the combined mRNA and miRNA expression in the patient's biological sample, e.g., blood, from that of the reference correlate with a diagnosis of a type of lung cancer. In one embodiment, significant changes in the combined mRNA and miRNA expression in the patient's biological sample, e.g., blood, from that of the reference correlate with a diagnosis of a non-cancerous condition, such as COPD, benign lung lesions or nodules. In one embodiment, significant changes in the combined mRNA and miRNA expression in the patient's biological sample, e.g., blood, from that of the reference correlate with a diagnosis of another disease. Further these compositions are useful to provide a supplemental or original diagnosis in a subject having lung nodules of unknown etiology.

In one embodiment of the compositions described above, the reference control is a non-healthy control (NHC). In other embodiments, the reference control may be any class of controls as described above. A composition containing polynucleotides or oligonucleotides that hybridize to the members of the selected combined gene and miRNA expression profile is desirable not only for diagnosis, but for monitoring the effects of surgical or non-surgical therapeutic treatment to determine if the positive effects of resection/chemotherapy are maintained for a long period after initial treatment. These profiles also permit a determination of recurrence or the likelihood of recurrence of a lung cancer, e.g., NSCLC, if the results demonstrate a return to the pre-surgery/pre-chemotherapy profiles. It is further likely that these compositions may also be employed for use in monitoring the efficacy of non-surgical therapies for lung cancer.

The compositions based on the genes and miRNA selected from Table 1, 2 and/or 3, optionally associated with detectable labels, can be presented in the format of a microfluidics card, a chip or chamber, or a kit adapted for use with the PCR, RT-PCR or Q PCR techniques described above. In one aspect, such a format is a diagnostic assay using TAQMAN® Quantitative PCR low density arrays. Preliminary results suggest the number of genes and miRNA required is compatible with these platforms. When a biological sample from a selected subject is contacted with the primers and probes in the composition, PCR amplification of targeted informative genes and miRNA in the expression profile from the subject permits detection of changes in expression in the genes and miRNA from that of a reference gene expression profile. Significant changes in the combined expression of the selected mRNA and miRNA in the patient's sample from that of the reference profile can correlate with a diagnosis of lung cancer. Similarly, when a biological sample from a post-surgical patent subject is contacted with the primers and probes in the composition, PCR amplification of targeted informative genes and miRNA selected from those of Table 1, 2 and/or 3 in the profile can be compared from that of the patient (or a similar patient) prior to surgery. Significant changes in the expression of the selected mRNA and miRNA in the patient's sample from that of the reference expression profile correlate with a positive effect of surgery, and/or maintenance of the positive effect.

The design of the primer and probe sequences is within the skill of the art once the particular mRNA and miRNA targets are selected. The particular methods selected for the primer and probe design and the particular primer and probe sequences are not limiting features of these compositions. A ready explanation of primer and probe design techniques available to those of skill in the art is summarized in U.S. Pat. No. 7,081,340, with reference to publically available tools such as DNA BLAST software, the Repeat Masker program (Baylor College of Medicine), Primer Express (Applied Biosystems); MGB assay-by-design (Applied Biosystems); Primer3 (Steve Rozen and Helen J. Skaletsky (2000) Primer3 on the WWW for general users and for biologist programmers and other publications. In general, optimal PCR primers and probes used in the compositions described herein are generally between 12 and 30, e.g., between 17 and 22 bases in length, and contain about 20-80%, such as, for example, about 50-60% G+C bases. Melting temperatures of between 50 and 80° C., e.g. about 50 to 70° C., are typically preferred.

The composition, which can be presented in the format of a microfluidics card, a microarray, a chip or chamber, employs the polynucleotide hybridization techniques described herein. When a biological sample from a selected patent subject is contacted with the hybridization probes in the composition, PCR amplification of targeted informative genes and miRNA in the expression profile from the patient permits detection and quantification of changes in expression in the genes and miRNA in the expression profile from that of a reference combined expression profile, e.g., a healthy control or a control with pulmonary disease, but no cancer, etc.

These compositions may be used to diagnose lung cancers, such as stage I or stage II NSCLC. Further these compositions are useful to provide a supplemental or original diagnosis in a subject having lung nodules of unknown etiology. The combined mRNA and miRNA expression profiles formed by targets selected from Table 1, 2 and/or 3 or subsets thereof are distinguishable from an inflammatory gene expression profile.

Classes of the reference subjects can include a smoker with malignant disease, a smoker with non-malignant disease, a former smoker with non-malignant disease, a healthy non-smoker with no disease, a non-smoker who has chronic obstructive pulmonary disease (COPD), a former smoker with COPD, a subject with a solid lung tumor prior to surgery for removal or same; a subject with a solid lung tumor following surgical removal of the tumor; a subject with a solid lung tumor prior to therapy for same; and a subject with a solid lung tumor during or following therapy for same. Selection of the appropriate class depends upon the use of the composition, i.e., for original diagnosis, for prognosis following therapy or surgery or for specific diagnosis of disease type, e.g., AC vs. LSCC.

### Diagnostic Methods

All of the above-described compositions provide a variety of diagnostic tools which permit a blood-based, non-invasive assessment of disease status in a subject. Use of these compositions in diagnostic tests, which may be coupled with other screening tests, such as a chest X-ray or CT scan, increase diagnostic accuracy and/or direct additional testing. In other aspects, the diagnostic compositions and tools described herein permit the prognosis of disease, monitoring response to specific therapies, and regular assessment of the risk of recurrence. The methods and use of the compositions described herein also permit the evaluation of changes in diagnostic combined mRNA and miRNA levels or profiles pre-therapy, pre-surgery and/or at various periods during therapy and post therapy samples and identifies a combined expression profile or signature that may be used to assess the probability of recurrence.

In one embodiment, a method of diagnosing or detecting or assessing a condition in a mammalian subject comprises detecting in a biological sample of the subject, or from a combined mRNA and miRNA expression profile generated from the sample, the expression level of the target mRNA and miRNA nucleic acid sequences identified in Table 1, 2 and/or 3; and comparing the combined mRNA and miRNA expression levels or profile in the subject's sample to a reference standard. A change in expression of the subject's sample profile from that of the reference standard indicates a diagnosis or prognosis of a condition mentioned above, depending upon the selection of the reference standard. In certain embodiments, the condition is a lung cancer, chronic obstructive pulmonary disease (COPD), or benign lung nodules. These methods may be employed using the biological samples discussed above. In certain embodiments, the biological sample is whole blood, peripheral blood mononuclear cells, plasma and serum.

As discussed above, this method involves in certain embodiments, measuring the expression level of a combination of one or more specified mRNA and one or more specified miRNA in the subject's sample. In other embodiments, the detecting, measuring or comparing steps of the method are repeated multiple times. For example, in certain embodiments, the mRNA and miRNA levels are detected or measured in a series of samples of said subject taken at different times. This permits identification of a pattern of altered expression of said combined mRNA and miRNA from a selected reference standard.

In still other embodiments, the detecting or measuring step involves contacting a biological sample from the subject with a diagnostic reagent, such as those described above that identifies or measures the target mRNA and miRNA expression levels in the sample. In certain embodiments, the contacting step involves or comprises forming a direct or indirect complex in said biological samples between a diagnostic reagent for said mRNA or miRNA and the mRNA or miRNA in the sample. Thereafter, the method measures a level of the complex in a suitable assay, such as described herein.

In certain embodiments of these methods, the mRNA and miRNA targets forming the combined profile are differentially expressed in two or more of the conditions selected from no lung disease with no history of smoking, no lung disease with a history of smoking, lung cancer, chronic obstructive pulmonary disease (COPD), benign lung nodules, lung cancer prior to tumor resection, and lung cancer following tumor resection. Depending on the conditions being assessed by the methods, the reference standard is obtained from a reference subject or reference population such as (a) a reference human subject or population having a non-small cell lung cancer (NSCLC); (b) a reference human subject or population having COPD, (c) a reference human subject or population who are healthy and have never smoked, (d) a reference human subject or population who are former smokers or current smokers with no disease; (e) a reference human subject or population having benign lung nodules; (f) a reference human subject or population following surgical removal of an NSCLC tumor; (g) a reference human subjects or population prior to surgical removal of an NSCLC tumor; and (h) the same subject who provided a temporally earlier biological sample.

The diagnostic compositions and methods described herein provide a variety of advantages over current diagnostic methods. Among such advantages are the following. As exemplified herein, subjects with adenocarcinoma or squamous cell carcinoma of the lung, the two most common types of lung cancer, are distinguished from subjects with non-malignant lung diseases including chronic obstructive lung disease (COPD) or granuloma or other benign tumors. These methods and compositions provide a solution to the practical diagnostic problem of whether a patient who presents at a lung clinic with a small nodule has malignant disease. Patients with an intermediate-risk nodule would clearly benefit from a non-invasive test that would move the patient into either a very low-likelihood or a very high-likelihood category of disease risk. An accurate estimate of malignancy based on a genomic profile (i.e. estimating a given patient has a 90% probability of having cancer versus estimating the patient has only a 5% chance of having cancer) would result in fewer surgeries for benign disease, more early stage tumors removed at a curable stage, fewer follow-up CT scans, and reduction of the significant psychological costs of worrying about a nodule. The economic impact would also likely be significant, such as reducing the current estimated cost of additional health care associated with CT screening for lung cancer, i.e., $116,000 per quality adjusted life-year gained. A non-invasive test that has a sufficient sensitivity and specificity would significantly alter the post-test probability of malignancy and thus, the subsequent clinical care.

A desirable advantage of these methods over existing methods is that they are able to characterize the disease state from a minimally-invasive procedure, i.e., by taking a blood sample. In contrast current practice for classification of cancer tumors from gene expression profiles depends on a tissue sample, usually a sample from a tumor. In the case of very small tumors a biopsy is problematic and clearly if no tumor is known or visible, a sample from it is impossible. No purification of tumor is required, as is the case when tumor samples are analyzed. A recently published method depends on brushing epithelial cells from the lung during bronchoscopy, a method which is also considerably more invasive than taking a blood sample, and applicable only to lung cancers, while the methods described herein are generalizable to any cancer. Blood samples have an additional advantage, which is that the material is easily prepared and stabilized for later analysis, which is important when mRNA or miRNA is to be analyzed.

### EMBODIMENTS

In one embodiment, a multi-analyte composition for the diagnosis of lung cancer comprises (a) a ligand selected from a nucleic acid sequence, polynucleotide or oligonucleotide capable of specifically complexing with, hybridizing to, or identifying an mRNA gene transcript from a mammalian biological sample; and (b) an additional ligand selected from a nucleic acid sequence, polynucleotide or oligonucleotide capable of specifically complexing with, hybridizing to, or identifying an miRNA from a mammalian biological sample. Each ligand and additional ligand binds to a different gene transcript or miRNA and the combined expression levels of the gene transcripts and miRNA identified form a characteristic profile of a lung cancer or stage of lung cancer.

In another embodiment, the gene transcripts and miRNA of the above composition are selected from Table 1. In another embodiment, the gene transcripts and miRNA of the composition are selected from rankings 1 to 119 of Table 1. In another embodiment, the gene transcripts and miRNA of the above composition are selected from all targets of Table 1. In another embodiment, the gene transcripts and miRNA of the above composition are selected from some or all targets of Table 2. In another embodiment, the gene transcripts and miRNA of the composition are selected from some or all targets of Table 3.

In still another embodiment, each said ligand of the composition is an amplification nucleic acid primer or primer pair that amplifies and detects a nucleic acid sequence of said gene transcript or miRNA. In another embodiment, the ligand is a polynucleotide probe that hybridizes to the gene's mRNA or miRNA nucleic acid sequence. In another embodiment, the composition contains an antibody or fragment of an antibody, each ligand being specific for at least one mRNA or one miRNA of Table 1, 2 or 3.

In another embodiment, the composition further comprises a substrate upon which said ligands are immobilized. In another embodiment, the composition comprises a microarray, a microfluidics card, a chip, a chamber or a complex of multiple probes. In another embodiment, the composition comprises a kit comprising multiple probe sequences, each said probe sequence capable of hybridizing to one mRNA and one miRNA of the mRNA and miRNA ranked from 1 to 119 of Table 1, or all targets of Table 1, or some or all targets of Table 2 and/or some or all targets of Table 3. In another embodiment, the kit comprises additional ligands that are capable of hybridizing to the same mRNA or miRNA. In still another embodiment, the kit comprises multiple said ligands, which each comprise a polynucleotide or oligonucleotide primer-probe set. In another embodiment, the kit comprises both primer and probe, wherein each said primer-probe set amplifies a different gene transcript or miRNA.

In another embodiment, the composition contains one or more polynucleotide or oligonucleotide or ligand associated with a detectable label.

In another embodiment, the composition enables detection of changes in expression, expression level or activity of the same selected genes and miRNA in the whole blood of a subject from that of a reference or control, wherein said changes correlate with an initial diagnosis of a lung cancer, a stage of lung cancer, a type or classification of a lung cancer, a recurrence of a lung cancer, a regression of a lung cancer, a prognosis of a lung cancer, or the response of a lung cancer to surgical or non-surgical therapy. In another embodiment, the lung cancer is a non-small cell lung cancer.

In another embodiment, the composition enables detection of changes in expression in the same selected genes in the blood of a subject from that of a reference or control, wherein said changes correlate with a diagnosis or evaluation of a lung cancer.

In another embodiment, the diagnosis or evaluation comprise one or more of a diagnosis of a lung cancer, a diagnosis of a stage of lung cancer, a diagnosis of a type or classification of a lung cancer, a diagnosis or detection of a recurrence of a lung cancer, a diagnosis or detection of a regression of a lung cancer, a prognosis of a lung cancer, or an evaluation of the response of a lung cancer to a surgical or non-surgical therapy. In one embodiment of the composition, the ligand is an RNA primer.

In another embodiment, the composition is a kit or microarray comprising at least two ligands, at least one ligand identifying an mRNA transcript of a selected gene which has a modification in expression when the subject has lung cancer and at least a second ligand identifying an miRNA that has a change in expression level when the subject has lung cancer.

Still another embodiment of the invention is a method for diagnosing the existence or evaluating a lung cancer in a mammalian subject comprising identifying in the biological fluid of a mammalian subject changes in the expression of gene transcripts and miRNA selected from rankings 1 to 119 of Table 1, all targets of Table 1, some or all targets of Table 2, and/or some or all targets of Table 3, and comparing said subject's mRNA and miRNA expression levels with the levels of the same mRNA and miRNA in the same biological sample from a reference or control, wherein changes in expression of the subject's mRNA and miRNA genes from those of the reference correlates with a diagnosis or evaluation of a lung disease or cancer.

In one embodiment, the method uses the multi-analyte composition described herein. In another embodiment, the method permits a diagnosis or evaluation to comprise one or more of a diagnosis of a lung cancer, a benign lung nodule, a diagnosis of a stage of lung cancer, a diagnosis of a type or classification of a lung cancer, a diagnosis or detection of a recurrence of a lung cancer, a diagnosis or detection of a regression of a lung cancer, a prognosis of a lung cancer, or an evaluation of the response of a lung cancer to a surgical or non-surgical therapy.

In another embodiment, the diagnosis or evaluation of the method comprises the diagnosis of an early stage of lung cancer.

In another embodiment the method permits detection of changes that comprise a combination of an upregulation or down-regulation of one or more selected gene transcripts in comparison to said reference or control and an upregulation or a downregulation of one or more selected miRNA in comparison to said reference or control. In another embodiment, the gene transcripts and miRNA used in the method are selected from among those listed in Table 1, 2 and/or 3. In another embodiment, the lung cancer is stage I or II non-small cell lung cancer.

In still further embodiments, the subject has undergone surgery for solid tumor resection or chemotherapy; and wherein said reference or control comprises the same selected gene transcripts and miRNA from the same subject pre-surgery or pre-therapy; and wherein changes in expression of said selected gene transcripts and miRNA correlate with cancer recurrence or regression. In still other embodiments, the reference or control comprises at least one reference subject, said reference subject selected from the group consisting of: (a) a smoker with malignant disease, (b) a smoker with non-malignant disease, (c) a former smoker with non-malignant disease, (d) a healthy non-smoker with no disease, (e) a non-smoker who has chronic obstructive pulmonary disease (COPD), (f) a former smoker with COPD, (g) a subject with a solid lung tumor prior to surgery for removal of same; (h) a subject with a solid lung tumor following surgical removal of said tumor; (i) a subject with a solid lung tumor prior to therapy for same; and (j) a subject with a solid lung tumor during or following therapy for same; wherein said reference or control subject (a)-(j) is the same test subject at a temporally earlier timepoint. In other embodiments, the reference mRNA or miRNA standard is a mean, an average, a numerical mean or range of numerical means, a numerical pattern, a graphical pattern or an combined mRNA and miRNA expression profile derived from a reference subject or reference population.

In other embodiments, the biological sample used in the method is whole blood, serum or plasma.

In yet a further embodiment, the method comprises contacting the biological sample from the subject with a diagnostic reagent that complexes with and measures the selected mRNA expression levels in the sample and contacting the biological sample from the subject with a diagnostic reagent that complexes with and measures the miRNA expression levels in the sample, wherein the combined changes in the expression levels is diagnostic of a cancer or stage thereof.

In still another embodiment, the selected miRNA and mRNA are differentially expressed in two or more of the conditions selected from no lung disease with no history of smoking, no lung disease with a history of smoking, lung cancer, chronic obstructive pulmonary disease (COPD), benign lung nodules, lung cancer prior to tumor resection, and lung cancer following tumor resection.

In another embodiment, a method of generating a diagnostic reagent comprising forming a disease classification profile comprising detecting combined changes in expression of selected mRNA and miRNA sequences characteristic of the disease in a sample of a mammalian subject's biological fluid.

The following examples are provided for the purpose of illustration only and the invention should in no way be construed as being limited to these examples but rather should be construed to encompass any and all variations that become evident as a result of the teaching provided herein.

### Example 1: Sample Size Calculation

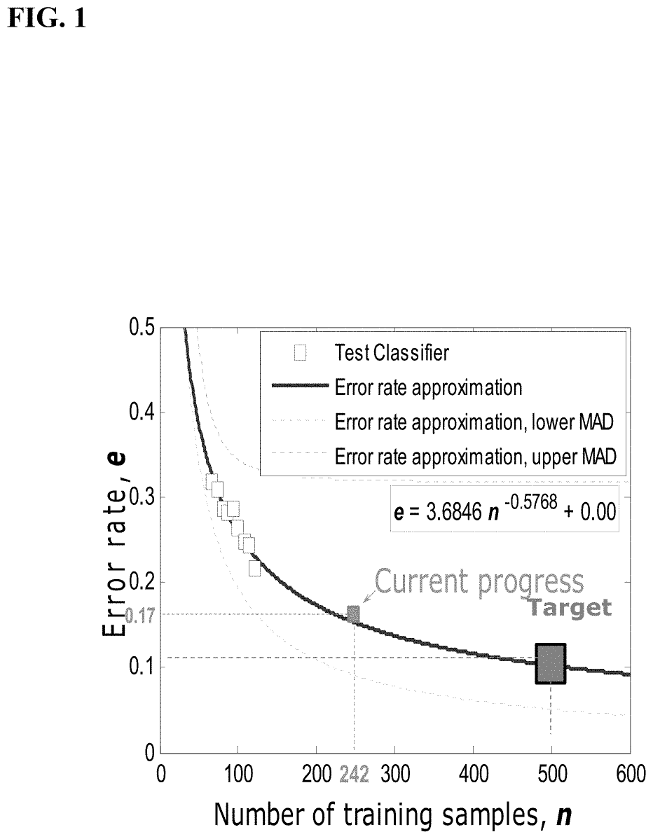

This calculation is based on the PAXgene data described in FIG. 1. We used the data from the current PAXgene dataset of 23 cancer patients and 25 controls to project the sample size that would be needed to reach the desired 90% accuracy on a test set. We randomly selected training sets of different sizes varying between 24 and 44 samples, corresponding to 50 to 90% of all the samples. The sample size was progressively increased by increments of two to allow the addition of one cancer and one control sample at each step. For every given sample size, 50 re-samplings were done.

A t-test was then performed on each training set to identify the top 100 genes ranked by p-values. The gene lists were further reduced by removing any low expressors (expression that did not exceed twice the average background level for all the samples in the cancer and non-cancer groups).

The remaining 58 genes were then used to cluster all the samples including those initially held out for testing purposes. We used standardized Euclidean distance and complete linkage as the metrics for hierarchical clustering. The tree was partitioned into two clusters by creating a single horizontal cut through the tree to identify two clusters (36), one with the majority cancers and the other the majority non-cancers. The hold-out samples were assigned to one of the two clusters where the cancer cluster is defined as the cluster that contains the majority of the cancer samples.

The number of held-out test samples that were misclassified was used to calculate the error rate (e=# misclassified/total). We then calculated the median error rate and the median absolute deviation for the 50 iterations at each specific training set size. Similar to the process described previously, a power function curve was fit into the data from the median error rate and we obtained the equation of the line in order to estimate the required number of samples needed for training to achieve the desired 90% accuracy on the held-out test samples as shown in FIG. 1. Our calculations indicate that 90% classification accuracy on a new test set can be achieved by using a training set containing approximately 500 samples split between patients and controls.

### Example 2—RNA Purification and Quality Assessment

RNA purification for gene and miRNA array processing are carried out using standardized procedures as a regular service by the Genomics Core. PAXgene RNA is prepared using a standard commercially available kit from Qiagen™ that allows simultaneous purification of mRNA and miRNA. The resulting RNA is used for mRNA or miRNA profiling.

The RNA quality is determined using a Bioanalyzer. Only samples with RNA Integrity numbers >7.5 were used. A constant amount (100 ng) of total RNA was amplified (aRNA) using the Illumina-approved RNA amplification kit (Epicenter). This procedure provides sufficient amplified material for multiple repeats of gene and miRNA expression. RNA amounts as low as 10 ng can be used if smaller samples are to be acquired at a later date with alternative collection systems.

### Example 3—Data Pre-Processing, Array Quality Control, Probe Filtering

Array data is processed by Illumina's Bead Studio and expression levels of signal and control probes are exported for analysis. To reduce experimental noise, data is filtered by removing non-informative probes (probes not detected in >95% of all samples) and probes that do not change at least 1.2-fold between any two samples. The expression levels are then quantile normalized. These procedures result in quantile-normalized data with non-informative probe data removed.

After each hybridization batch, we computed gene-wise global correlation as a median Spearman correlation across all microarrays using expression levels of all signal probes (>40,000) and calculate the median absolute deviation of the global correlation. For each microarray a median Spearman correlation is computed against all other arrays and arrays whose median correlation differs from global correlation by more than eight absolute deviations are marked as outliers and not used for further analysis, typically for <1% of PAXgene samples. The further identification of outliers is done through multivariate statistics such as general or robust principal components (PCA) plots and multi-dimensional scaling.

For miRNA Expression, we chose the OpenArray platform from ABI (Life Sciences) for this study. The OpenArray nanofluidic PCR platform allows scientists to conduct up to 3,072 independent PCR analyses simultaneously and is already being used for clinical applications and uses a robotic station that eliminates variability. Additional platforms considered for this process are the nCounter System from Nanostring Technologies, Inc. (Seattle, Wash.). Briefly, this system utilizes a digital color-coded barcode technology. A color-coded molecular “barcode” is attached to a single target-specific probe for the target gene. The barcode hybridizes directly to the target molecule and can be individually counted without the need for amplification. A single molecule imaging with sets of such barcoded probes and controls permits detection and counting of many unique transcripts in a single reaction. See, e.g., the description of the NanoString Technology contained in the website, www.nanostringtechnology.com. For miRNA Data pre-processing and OpenArray quality control, total RNA is processed according to the ABI protocol using the OpenArray reagents purchased from ABI. Data from OpenArray are pre-processed using MATLAB as follows: the average cycle threshold (Ct) of the small nuclear RNAs, RNU44 and RNU48 (RNU_{avg}) are used as endogenous controls (housekeeping genes) to normalize the expression levels of the samples and compute relative amounts for each miRNA (ΔCt). Ct values are restricted to 24 as suggested by the manufacturer (and our facility), and the maximum ΔCt value will be equal to ΔCt₂₄ (where ΔCt₂₄=24−RNU_{avg}). ΔCt values exceeding ΔCt₂₄ are considered unreliable and will be floored to the ΔCt₂₄ value for the comparative analyses. The ΔCt value will then be converted to absolute expression levels by calculating 2^{ΔCt24-ΔCt}. All reactions are carried out in triplicate. All assays are carried out using highly standardized conditions. For statistical consideration, samples are collected from non-cancer patients with or without lung nodules and patients with lung cancer. Based on the results of our previous PBMC study, we assume that a better gene panel will be identified to distinguish the cancers from all non-cancers from 600 PAXgene samples (combining patients with or without lung nodules). The sample size and power estimations were based on this assumption.

In clinical practice, it will be more immediately important to distinguish cancers from patients with truly non-malignant nodules. Based on our previous experience, the potential gene panel for classifying cancers and non-malignant nodules will differ to some extent from that identified for classifying cancers and all non-cancers. There are several ways to determine genes panels for classification. One traditional way is the procedure we used for our preliminary PAXgene studies, by t-test as described for the preliminary PAXgene studies using the Benjamini and Hochberg, J. Royal Statis Soc., Series B, Vol. 57(1):289-300 (1995) adjusted p-value with p<0.05 and 50-100 genes with the lowest p values to be selected for Hierarchical clustering, but this is not effective for large datasets where we have instead successfully used SVM-RFE.

### Example 4—Supervised Classification for Gene Election

We have found the Support Vector machine with Recursive Feature Elimination (SVM-RFE) (see WO2010/054233) to be most successfully applied to develop gene expression classifiers that distinguish clinically-defined classes (e.g. cancer/non-cancer/benign nodules) that share many confounding similarities (smoking history, pulmonary disease, age, race etc). Unlike many other supervised methods, SVM has the advantage for biomarker selection since the genes are ranked by their contribution to the class separation so the most useful genes for the separation can be identified. The contributing genes are reduced by the iterative process of RFE to find the minimal number of genes that provide the most accurate class distinction. In addition, each sample is given a positive or negative score that assigns it to one class or another and that is a measure of how well that sample is identified with a particular class, as shown in FIG. 1. In our studies, positive is defined as cancer and negative is non-cancer. The higher the positive or the lower the negative score defines how well each sample is assigned to a particular class. The process is described in more detail below.

Sample classification is performed using SVM-RFE, with random, tenfold resampling and cross-validation repeated 10 times (yielding 100 gene-rankings). Each cross-validation iteration starts with the 1,000 genes most significant by t-test, and the number of genes is reduced by 10% at each feature elimination step. Final ranking of the genes is done using a Borda count procedure. Classification scores for each tested sample are recorded at each cross-validation and gene-reduction step, down to a single gene. The number of genes that yield the best accuracy is determined, and all genes associated with the points of maximal accuracy constitute the initial discriminator. This discriminator is then reduced as far as possible without loss of accuracy to arrive at the final discriminator. With SVM-RFE the cross-validation step is crucial to avoid over-fitting.

For validation procedures, to further ensure generality of the classifier, we withhold 25-30% of all patients from the analyses, thus forming an independent validation set. The independent validation samples are classified using the candidate genes derived from the analysis of the 70-75% of the samples in the training set. At each step the sensitivity and specificity of the discriminator Power calculations is reassessed to define the required endpoint.

A major strength and innovation of our classification strategy is to incorporate multiple data types, including mRNA and miRNA, in order to optimize discriminating power, and achieving synergies between these distinct levels of gene regulation. Such a multimodal analysis offers great potential for cancer diagnosis. Therefore, mRNA and miRNA are used both independently and as merged datasets to identify the best discriminators that use either only one type of data, or that yield benefit from merging all available information. Data from each platform is separately quantitated, normalized, and analyzed by the unsupervised classification techniques we previously applied to mRNA.

The data from each of these techniques are quantitative, differentially expressed features that are analyzed by t-test, and significant features for each type of data are further analyzed both separately and as a combined dataset by SVM-RFE. We anticipate that the most compact feature set contain some of both types of data. In particular, a single informative miRNA might be as informative as, and therefore replace, a number of mRNA species that it regulates. Sets of genes or miRNAs determined by SVM-RFE to be included in the discriminator can be further analyzed in order to identify common functions or pathways that differentiate any given two groups of samples being compared and have the potential to identify new therapeutic targets.

### Development and Implementation of the Diagnostic Algorithm:

Based on our previously published gene signature, we identify a signature of >30 gene probes, and/or less than 20 miRNA probes. Classification accuracies of mRNA and miRNA are assessed separately with each data type being normalized and processed separately. The OPENARRAY system allows us to develop customized arrays that can test candidate genes on a high-throughput platform. In addition the NANOSTING platform provides an easy, robust system for further testing and implementing a commercial test. Both the mRNA and the miRNA platforms ultimately result in a number that is a measure of how much of that entity is present in a sample. This means that the final data for classification can be combined into one matrix and used as a single classifier. Sample classes, analysis strategy and numbers of samples and their subtypes are summarized in Table 4.

TABLE 4
Summary of the Number of Samples Used For the Various Analyses.
Number of Samples Analyzed

| | | set A* | | | set B** |
|---|---|---|---|---|---|
| Comparison | Class | total | training | testing | total |
| LC vs. NOD | LC | 181 | 127 | 54 | |
| | NOD | 99 | 69 | 30 | |
| | total | 280 | 196 | 84 | 70 + 65 |
| LC vs. NOD + SC | LC | 181 | 127 | 54 | |
| | NOD + SC | 164 | 115 | 49 | |
| | total | 345 | 242 | 103 | 70 |

*(Set A) 345 samples were unambiguously assigned as Cancer (LC) or Control (NOD or SC) were used for training and testing.
**(Set B) 70 samples with indistinct phenotypes. These 70 samples include post lung resection samples and samples from nodule patients who later developed LC, so the status of the cancer signature was essentially unknown. The LC vs. NOD comparison also included 65 SC samples that were not used in training-testing, but were available for classification.

Of the 415 total samples in analysis, 345 samples had unambiguously assigned Cancer (LC) or Control (NOD or SC) labels (set A) and were used for training and testing purposes. The remaining 70 samples included samples with indistinct phenotypes (set B): post lung resection samples and samples from nodule patients who later developed LC and were used for further classification by the classifier developed on the 345 unambiguously assigned samples (clinically confirmed as case or control but not including post resection samples). Samples from both sets were randomly split into 70% for the training set (242 samples for Set A) and a set aside 30% for the testing set (103 samples for Set A).

The training set was used to find the best classifier by SVM with a 10-fold cross-validation routine using Radial Basis Function (RBF) kernel and forward feature selection (FFS) that at each step picked one best feature (gene or miRNA) which improved overall training accuracy. Alternatively, we tried using linear kernel and Recursive Feature Elimination (RFE), which we used successfully in the past 8, but forward feature selection with RBF kernel gave better accuracy on the preliminary training set. A classifier built for the number of features that provided the best training accuracy was then selected as a final classifier and applied to the independent set-aside testing set to estimate its unbiased accuracy.

Using the described classifier development process, we used three data sets to create three different classifiers for comparison: (1) using only mRNA data; (2) using only miRNA expression data, and (3) analyzing the combined mRNA and miRNA data. Each dataset/classification analysis resulted in a report based on the testing set performance and included accuracy, sensitivity, specificity and area under ROC-curve (AUC). The results are listed in Table 5.

TABLE 5
Preliminary Accuracies. Sensitivities and Specificities in Distinguishing
patients with lung cancer (LC) from patients with benign nodules
(NOD) and smoking controls without nodules (SC).*

| | | Total | | | | |
|---|---|---|---|---|---|---|
| | Data | target | Accu- | Sensi- | Speci- | |
| Comparison | Type | # | racy | tivity | ficity | AUC |
| LC vs. | mRNA | 161 | 81% | 92% | 60% | 0.86 |
| NOD | miRNA | 5 | 75% | 83% | 60% | 0.75 |
| | Both | 147 ** | 79% | 87% | 67% | 0.87 |
| LC vs. | mRNA | 151 | 79% | 78% | 80% | 0.88 |
| NOD + SC | miRNA | 26 | 71% | 69% | 73% | 0.77 |
| | Both | 145 *** | 83% | 81% | 84% | 0.88 |

*Data is presented for the analyses using only gene expression (mRNA), only miRNA expression and mRNA + miRNA expression (Both). NOD = nodules, SC = Smoking controls without nodules.
** Targets (all) from Table 2.
*** Targets (all) from Table 1.

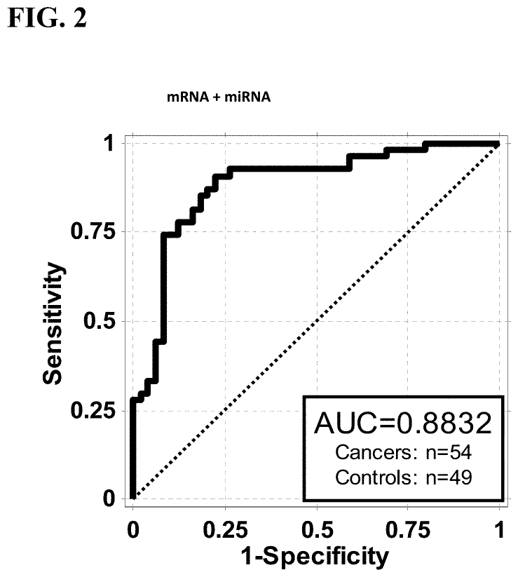

According to the table, the best accuracy was achieved by general Cancer vs all Controls classifier (83% accuracy) that used both mRNA and miRNA data at the same time (145 total features), which demonstrates advantage of using both platforms in the same classification. The ROC AUC for the combined classifier is shown in FIG. 2.

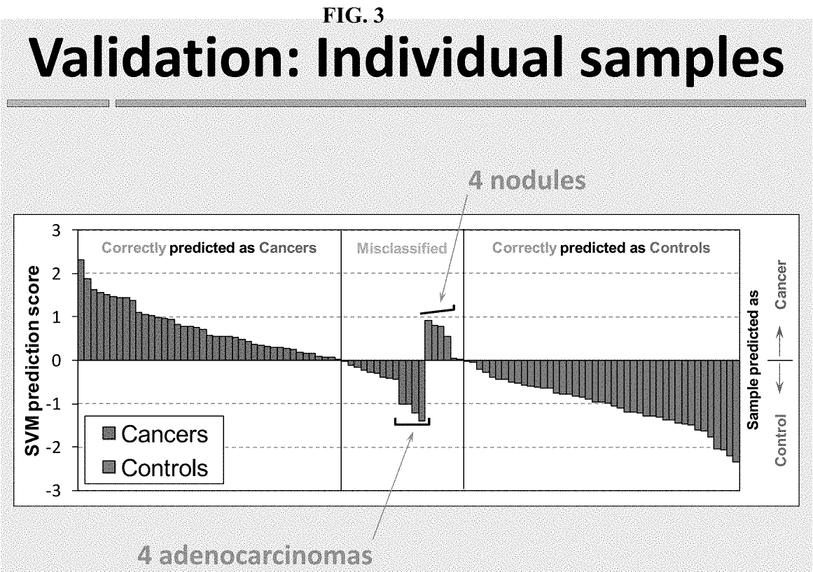

The individual scores for each sample from the independent testing set assigned by the classifier are shown in the SVM plot in FIG. 3, where each sample received a score assigned by the SVM classifier. Positive scores indicate classification as cancer and negative scores as a control. Each column represents a patient and the height of the column can be interpreted as a measure of the strength or the reliability of the classification. The classification shown uses the classical 0 point cutoff for classification. The graph shows a cutoff that maximizes sensitivity at 92.6% with Specificity at 73.5%.

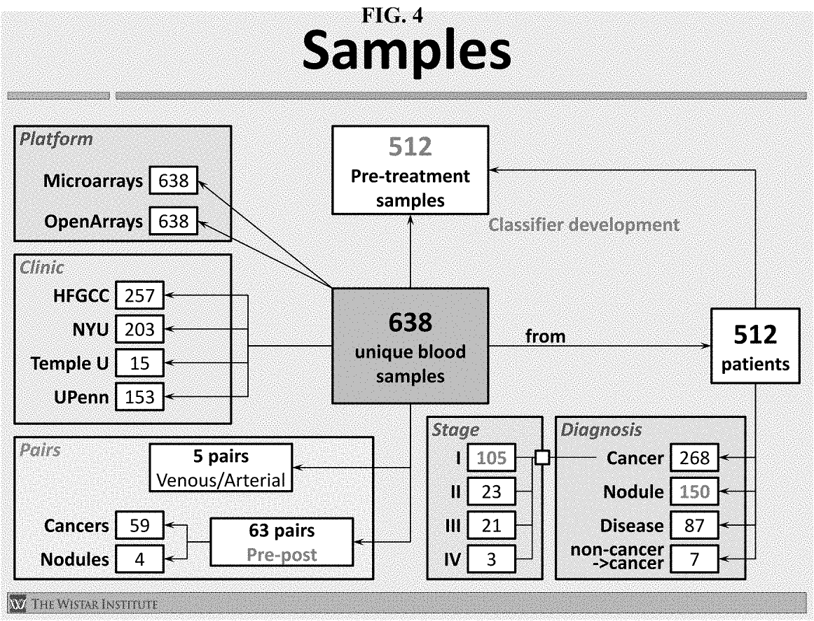

FIG. 4 shows preliminary results of this methodology: 345 samples were processed and analyzed using Illumina HT12v4 mRNA arrays and miRNAs on ABI OpenArray PCR platform. To ensure a completely independent testing set, 242 (70%) were training sets, and 103 (30%) were testing samples.

Each and every patent, patent application, including U.S. patent application Ser. No. 15/574,737, International Patent Application No. PCT/US2016/033232, U.S. provisional patent application No. 62/163,766, and publication, including websites cited throughout the disclosure, is expressly incorporated herein by reference in its entirety. While this invention has been disclosed with reference to specific embodiments, it is apparent that other embodiments and variations of this invention are devised by others skilled in the art without departing from the true spirit and scope of the invention. The appended claims include such embodiments and equivalent variations.

1. A multi-analyte composition for the diagnosis of lung cancer comprising
(a) a ligand selected from a nucleic acid sequence, polynucleotide or oligonucleotide capable of specifically complexing with, hybridizing to, or identifying an mRNA gene transcript from a mammalian biological sample; and
(b) an additional ligand selected from a nucleic acid sequence, polynucleotide or oligonucleotide capable of specifically complexing with, hybridizing to, or identifying an miRNA from a mammalian biological sample;
wherein each ligand and additional ligand binds to a different gene transcript or miRNA and the combined expression levels of the gene transcripts and miRNA identified form a characteristic profile of a lung cancer or stage of lung cancer.


SEQUENCE LISTING (292 sequences)

SEQ ID NO: 1 - RNA, 22 nt, Homo sapiens
caaagaauucuccuuuugggcu

SEQ ID NO: 2 - DNA, 50 bp, Homo sapiens
gattgcagggtccgccttctcaaaccccacttcctggaccacatcatcca

SEQ ID NO: 3 - RNA, 21 nt, Homo sapiens
uaaagugcugacagugcagau

SEQ ID NO: 4 - RNA, 22 nt, Homo sapiens
cagugcaauguuaaaagggcau

SEQ ID NO: 5 - DNA, 50 bp, Homo sapiens
gtcccaaagagtttgatgaggccctccacacctgcggcccaatccaaggt

SEQ ID NO: 6 - RNA, 21 nt, Homo sapiens
uaccacaggguagaaccacgg

SEQ ID NO: 7 - DNA, 50 bp, Homo sapiens
tcaacgccaggaatcatgaagagacttctgcttttcaacccccaccctcc

SEQ ID NO: 8 - DNA, 50 bp, Homo sapiens
tctggaggctgggaagtccaagatcaaggcgtcagaagattcattgtctg

SEQ ID NO: 9 - RNA, 22 nt, Homo sapiens
uagcuuaucagacugauguuga

SEQ ID NO: 10 - DNA, 50 bp, Homo sapiens
gcctgaggtgacagacagggcaggtggtaacaaaaccgttgaacctccca

SEQ ID NO: 11 - DNA, 50 bp, Homo sapiens
aagctgttgaaggtgagggtggtgtacgaagtgccactgttcctgtaagc

SEQ ID NO: 12 - DNA, 50 bp, Homo sapiens
agaaggagggtttctggctgtggttctaaatggagccccaggaagctgcc

SEQ ID NO: 13 - DNA, 50 bp, Homo sapiens
cactgtcgtccttcctcagagggcctcacgccaaacaaacggccttttcg

SEQ ID NO: 14 - DNA, 50 bp, Homo sapiens
gctaacatccattccctttcataccaccattttcaccctgtttcttcccc

SEQ ID NO: 15 - DNA, 50 bp, Homo sapiens
ggtccaggtgaatctcgtcataagtgatctcaggctctcacaggatccgg

SEQ ID NO: 16 - DNA, 50 bp, Homo sapiens
aaactcaaggactgcgtgaccgacacaatgacccccgaggagacagaggc

SEQ ID NO: 17 - DNA, 50 bp, Homo sapiens
ccttgctgcctacccttttctctcctctggttctcaacctcaacgagttc

SEQ ID NO: 18 - DNA, 50 bp, Homo sapiens
ccaaacactctccctacccattcctgccagctctgcctccttttcaactc

SEQ ID NO: 19 - DNA, 50 bp, Homo sapiens
caggttgcaatatgaggacttctctgtctcctctgaagcctgggacactg

SEQ ID NO: 20 - DNA, 50 bp, Homo sapiens
gaaagaaatgagcagctttggataatgacgacagcaacccgaagacaggg

SEQ ID NO: 21 - DNA, 50 bp, Homo sapiens
gctcgtgtgctacaatggcagagttgagcagtggtgacaaaccatgcgac

SEQ ID NO: 22 - DNA, 50 bp, Homo sapiens
gccaagtgccatttggggtcagcatcctcgtttcaacacagtgtgctctc

SEQ ID NO: 23 - DNA, 50 bp, Homo sapiens
gtcagtccaaggaggtatgttcttccacaacagccttctcagcctctgct

SEQ ID NO: 24 - DNA, 50 bp, Homo sapiens
cagaagagggagacctggagaccgttacgacggcatggttggtttcagtg

SEQ ID NO: 25 - DNA, 50 bp, Homo sapiens
gcttgctgctttctggctaatgaaagccaaggactatccagcacacacag

SEQ ID NO: 26 - DNA, 50 bp, Homo sapiens
gcaggtcatgcacacagttttgataaagggcagtaacaagtattggggcc

SEQ ID NO: 27 - DNA, 50 bp, Homo sapiens
gactggcaaggtttcctagagctctacttacagaacagccctgaggcctg

SEQ ID NO: 28 - DNA, 50 bp, Homo sapiens
gcctgccggatgatgaatggcatgaagctgagtggccgagagattgacgt

SEQ ID NO: 29 - RNA, 23 nt, Homo sapiens
agcuacauugucugcuggguuuc

SEQ ID NO: 30 - DNA, 50 bp, Homo sapiens
tgtctgtcattgtggcccgtttcacactgtctctatatctgtttcccctg

SEQ ID NO: 31 - DNA, 50 bp, Homo sapiens
agtcttcatctgtccgacaagttcactcgcctcggttgcggacctaggac

SEQ ID NO: 32 - DNA, 50 bp, Homo sapiens
gccgcctcgcaagctcttgttttctaaccccaccttctgggagccgtgtt

SEQ ID NO: 33 - DNA, 50 bp, Homo sapiens
gtcatgatctgctcggaatcctcctgctaaagaaggctctgggcgtgagc

SEQ ID NO: 34 - RNA, 21 nt, Homo sapiens
aauggcgccacuaggguugug

SEQ ID NO: 35 - DNA, 50 bp, Homo sapiens
ggagtatgggagagagggactgccacacagaagctgaagacaacacctgc

SEQ ID NO: 36 - DNA, 50 bp, Homo sapiens
ccaccccattcggttcttctgcctgaccttcaaatgcccatgttggcctt

SEQ ID NO: 37 - RNA, 21 nt, Homo sapiens
uccgguucucagggcuccacc

SEQ ID NO: 38 - RNA, 23 nt, Homo sapiens
aaaagugcuuacagugcagguag

SEQ ID NO: 39 - DNA, 50 bp, Homo sapiens
agactcctccagaccaggaaccccagaaggagacagagcctgccacatcc

SEQ ID NO: 40 - DNA, 50 bp, Homo sapiens
ccggaaagtctaccaagctgtgcggcacaataaagccacggaaaacaagg

SEQ ID NO: 41 - DNA, 50 bp, Homo sapiens
cacctgtgggcagtgggcagtgtcttggtgaaagggagcggatactactt

SEQ ID NO: 42 - DNA, 50 bp, Homo sapiens
gaggccaggctgaaatgtcatatctgaaggaagaaagcagcagctggaca

SEQ ID NO: 43 - DNA, 50 bp, Homo sapiens
cagcgttaatcctgtatggccaggaaactgagtagactcctgtgtaaccc

SEQ ID NO: 44 - DNA, 50 bp, Homo sapiens
ctgatctcagtgtctggtttgctgggtacccttctgctcatcatcctgac

SEQ ID NO: 45 - RNA, 17 nt, Homo sapiens
ucucgcuggggccucca

SEQ ID NO: 46 - DNA, 50 bp, Homo sapiens
atcgggaggacctgtatgcctgaccgtttccctgcctcctgcttcagcct

SEQ ID NO: 47 - DNA, 50 bp, Homo sapiens
ggtgaccagcagagtggttatgggaaggtatccaggcgaggtggtcatca

SEQ ID NO: 48 - DNA, 50 bp, Homo sapiens
taagattgctagggaaaagggccctatgtgtcaggcctctgagcccaagc

SEQ ID NO: 49 - DNA, 50 bp, Homo sapiens
agctgccctcattccgacttcagaaaatcgaagcagctggcgcctcccct

SEQ ID NO: 50 - RNA, 22 nt, Homo sapiens
ggauaucaucauauacuguaag

SEQ ID NO: 51 - DNA, 50 bp, Homo sapiens
cctctcgcctggaggatctgtgccatcttggattgagaattgcagatgtg

SEQ ID NO: 52 - DNA, 50 bp, Homo sapiens
ttcaccatcgtcttcaatgcccatgagcctttccgccggggtacaggtgt

SEQ ID NO: 53 - DNA, 50 bp, Homo sapiens
gaatccgatggtcctcgaaacatggaaagtctgctgtcacgctgcacgcc

SEQ ID NO: 54 - DNA, 50 bp, Homo sapiens
tgccggaagtcactaccaaggatcgatacacatttaggaaagccagcact

SEQ ID NO: 55 - RNA, 23 nt, Homo sapiens
caaagugcucauagugcagguag

SEQ ID NO: 56 - DNA, 50 bp, Homo sapiens
ggagagggtgacctggctgctggtttaccactgtaccaacatctctggag

SEQ ID NO: 57 - DNA, 50 bp, Homo sapiens
gggcttttactttggagcactctgtgtgaagctgtttggtggaacccatg

SEQ ID NO: 58 - DNA, 50 bp, Homo sapiens; identical to SEQ ID NO: 57

SEQ ID NO: 59 - DNA, 50 bp, Homo sapiens
cctgaactgatgggtttctccagagggaattgcagagtactggctgatgg

SEQ ID NO: 60 - RNA, 22 nt, Homo sapiens
gcugacuccuaguccagggcuc

SEQ ID NO: 61 - DNA, 50 bp, Homo sapiens
gcttcttacctgtgcgggagcgaaaaagctgggcttcaacatggcaggtc

SEQ ID NO: 62 - RNA, 22 nt, Homo sapiens
ugagguaggagguuguauaguu

SEQ ID NO: 63 - DNA, 50 bp, Homo sapiens
cactctatgggaaactcttcagcacctacctgcgccccccacacacctct

SEQ ID NO: 64 - DNA, 50 bp, Homo sapiens
cccagccctagatgtatccaagccctcctaccctcaccagttatttctgg

SEQ ID NO: 65 - DNA, 50 bp, Homo sapiens
ctccaaatgtcaaaggcaagctgggcatcatgatctggcataaagaaccc

SEQ ID NO: 66 - DNA, 50 bp, Homo sapiens
gcccagggccgccctagcaacttcctgtacatatgactgtaaaatggtaa

SEQ ID NO: 67 - RNA, 23 nt, Homo sapiens
uaaagugcuuauagugcagguag

SEQ ID NO: 68 - DNA, 50 bp, Homo sapiens
ccccgagttttgcccatatcaggacagtggctccttctcactcccctttc

SEQ ID NO: 69 - DNA, 50 bp, Homo sapiens
gcggcacagtcccacttccccatctccccaagtaggtggtgttagaaaac

SEQ ID NO: 70 - DNA, 50 bp, Homo sapiens
gaaagcggcctcatgaaggggaagccaagggtgccgagaccacaaagcgc

SEQ ID NO: 71 - DNA, 50 bp, Homo sapiens
agtcgtccttccctggtgcgcagcccaggcctgtgggtccagcctcaccc

SEQ ID NO: 72 - DNA, 50 bp, Homo sapiens
atggccatgacccagaagtatgaggagcatgtgcgggagcagcaggctca

SEQ ID NO: 73 - DNA, 50 bp, Homo sapiens
gcctgagggaccgcagactcgtcgggctgctttctgatgagaggattaac

SEQ ID NO: 74 - DNA, 50 bp, Homo sapiens
ggaaagtgaagatgcagagttactgtggcgtttggcacgggcatcacgtg

SEQ ID NO: 75 - DNA, 50 bp, Homo sapiens
accgatctttctctgtctcaccaacctgacaaaaaaggtgtgccaaggga

SEQ ID NO: 76 - DNA, 50 bp, Homo sapiens
acgatgccagactcatgtttggagatggaactcagctggtggtgaagccc

SEQ ID NO: 77 - DNA, 50 bp, Homo sapiens
cctcaaggagatgcctctggtccaggctttgtaaacttgggccttccagc

SEQ ID NO: 78 - DNA, 50 bp, Homo sapiens
gtagcactgttctggttctgtttgcacgccagtggggagagaataaagag

SEQ ID NO: 79 - DNA, 50 bp, Homo sapiens
gggcagtacagggccagatccacggcaggcacagggcaaagccaggccca

SEQ ID NO: 80 - DNA, 50 bp, Homo sapiens
ccaaggaatgcactaagccttcagtctttttagactgacagtactggcag

SEQ ID NO: 81 - DNA, 50 bp, Homo sapiens
ctatacccattcccaggcctaagccagcctctccctcctgacagtgccca

SEQ ID NO: 82 - DNA, 50 bp, Homo sapiens
gaggcatgggccaggtaaaaattgggcctagagtgaagactgtgctgtcg

SEQ ID NO: 83 - DNA, 50 bp, Homo sapiens
ggctggggtgagggctggtggttggtgaaagccattcttagttgtgtctc

SEQ ID NO: 84 - RNA, 22 nt, Homo sapiens
aauugcacgguauccaucugua

SEQ ID NO: 85 - DNA, 50 bp, Homo sapiens
gtcagatctcccctccaccagccaggatcctccttctagctcatctgtag

SEQ ID NO: 86 - DNA, 50 bp, Homo sapiens
gtgagccaaaatggcgctactgcactccagaccggggacagagtgagact

SEQ ID NO: 87 - RNA, 18 nt, Homo sapiens
gucccuguucaggcgcca

SEQ ID NO: 88 - DNA, 50 bp, Homo sapiens
aggagagaggtttgagttctgggtatcctccctttctgtaacagcctcaa

SEQ ID NO: 89 - RNA, 21 nt, Homo sapiens
cauuauuacuuuugguacgcg

SEQ ID NO: 90 - DNA, 50 bp, Homo sapiens
ctttggtccggcaacttcaacaacagctctctaatacccagccacagccc

SEQ ID NO: 91 - DNA, 50 bp, Homo sapiens
ccagccatcccattactgggtaggtacccaaatcatgctgctataaagac

SEQ ID NO: 92 - DNA, 50 bp, Homo sapiens
cccagggcattcagggctggttcagacaccattattgtgagcagcaaagc

SEQ ID NO: 93 - DNA, 50 bp, Homo sapiens
ccgccggtgccatatgatttagaggaagatgcaggctggtcactgctccc

SEQ ID NO: 94 - DNA, 50 bp, Homo sapiens
tcaagtcaaccctgagcagtatggggatgagtgatgccttcagccaaagc

SEQ ID NO: 95 - DNA, 50 bp, Homo sapiens
cataccaccctttggtgggaggaaactaaaaatatagcaaatgcagaacc

SEQ ID NO: 96 - RNA, 22 nt, Homo sapiens
ccuguucuccauuacuuggcuc

SEQ ID NO: 97 - DNA, 50 bp, Homo sapiens
ctagacgctggcactatggtcatggcggaggggacggcagtgctgaggcg

SEQ ID NO: 98 - DNA, 50 bp, Homo sapiens
cttttcgcagatgctgggaacgcagctctgctgccggcggggtggacaga

SEQ ID NO: 99 - DNA, 50 bp, Homo sapiens
tcgtccgcatggtgctgaatggctgaggaccttcccagtctccccagagt

SEQ ID NO: 100 - DNA, 50 bp, Homo sapiens
ggatccacatggtcttgagggttggcatgaggagggggaagcttttttga

SEQ ID NO: 101 - DNA, 50 bp, Homo sapiens
aatgctgcagttcctgatgagatcccccctctcgagggcgatgaggatgc

SEQ ID NO: 102 - DNA, 50 bp, Homo sapiens
gtggtagatcacttgaggtcaagagttgtgacaccagcctggccaacctg

SEQ ID NO: 103 - DNA, 50 bp, Homo sapiens
caaatatcatggaggtccctggattgaaaaaagagcctctcccactcctc

SEQ ID NO: 104 - DNA, 50 bp, Homo sapiens
ccctgcccccaaactggctaagacagctttcagttcctgactccccaact

SEQ ID NO: 105 - DNA, 50 bp, Homo sapiens
ctgagacgggcaagtggttgctccaggattactccctcctccaaaaaagg

SEQ ID NO: 106 - DNA, 50 bp, Homo sapiens
ctctggcctctgggtcccaccacccagccccccgtgtcagaacaatcttt

SEQ ID NO: 107 - DNA, 50 bp, Homo sapiens
tcctcgctaactgacattagcccattcaggtcttcacagcgctcatactg

SEQ ID NO: 108 - DNA, 50 bp, Homo sapiens
ccccaacttcgccctgcccacttgacttcaccaaatcccttcctggagac

SEQ ID NO: 109 - DNA, 50 bp, Homo sapiens
tgggggatttttcagtggaacccttgcccccaaatgtcgaccagccccca

SEQ ID NO: 110 - DNA, 50 bp, Homo sapiens
gtaaccggtctgcttttgcgtaagccaaacacctacccaaaaatgattcc

SEQ ID NO: 111 - DNA, 50 bp, Homo sapiens
ggccagttttatgaagctttggaaggcactatggacagaagctggtggac

SEQ ID NO: 112 - DNA, 50 bp, Homo sapiens
ctatggagagcagccgacaccccctcttacagccgtggatgtttcctgga

SEQ ID NO: 113 - DNA, 50 bp, Homo sapiens
ggccacggtgctggtgtcgctggtggagaacggccaggccccaaagacgt

SEQ ID NO: 114 - DNA, 50 bp, Homo sapiens
agtgtacctatttacagaaagattaaactgccacctgcgggcacattccc

SEQ ID NO: 115 - DNA, 50 bp, Homo sapiens
tactgaagtccctttgtgccagtggatcctggagggcctggggctgggca

SEQ ID NO: 116 - DNA, 50 bp, Homo sapiens
cgccgatatctctgccgggtgactagctgcttcctttctctctcgcgcgc

SEQ ID NO: 117 - DNA, 50 bp, Homo sapiens
cgactgccagggccttagactccacatgtccatttttgttcaggtatagc

SEQ ID NO: 118 - DNA, 50 bp, Homo sapiens
ctcgggcatccttcccagggttgggtcttacacaaatagaaggctcttgc

SEQ ID NO: 119 - RNA, 22 nt, Homo sapiens
uuauaaagcaaugagacugauu

SEQ ID NO: 120 - DNA, 50 bp, Homo sapiens
ccacagcctgtttctcccttggattccaagttccccatagaccattccct

SEQ ID NO: 121 - DNA, 50 bp, Homo sapiens
ccctcaactgcctttccaccacctatgatgttggggtttcagaaaaggtg

SEQ ID NO: 122 - DNA, 50 bp, Homo sapiens
ccacagacaccctaccgatagaacagtggctcagatcttacttgctcctg

SEQ ID NO: 123 - DNA, 50 bp, Homo sapiens
tacgagaccctccctgctgagatgcgcaaattcactccccagtacaaagg

SEQ ID NO: 124 - DNA, 50 bp, Homo sapiens
gtgctgttccactcttggctccagcagacccactgtcccagaaaagcctg

SEQ ID NO: 125 - DNA, 50 bp, Homo sapiens
cccagctgaacccgaggctaaagaagatgaggcaagagaaaatgtacccc

SEQ ID NO: 126 - DNA, 50 bp, Homo sapiens
cagcagatcagtgggatgagggagactgttcacctgctgtgtactcctgt

SEQ ID NO: 127 - DNA, 50 bp, Homo sapiens
cgtgggatctgcacacgtctttgtcagttgtggtcatgatcttagtcacc

SEQ ID NO: 128 - DNA, 50 bp, Homo sapiens
ggagtgtggcagacgttgtgcggttcatcagatccactgactgtgctcca

SEQ ID NO: 129 - DNA, 50 bp, Homo sapiens
tctgctggactgatgtcttctgcaggttgcagatcctgaccatgggctgc

SEQ ID NO: 130 - DNA, 50 bp, Homo sapiens
cgtcagtgccttcggactgtctatttgacctgcagtccagcctatggcct

SEQ ID NO: 131 - DNA, 50 bp, Homo sapiens
acttgtccacggtcctctcggtgaccctgttgggcagggccaagggacaa

SEQ ID NO: 132 - DNA, 50 bp, Homo sapiens
cgcctaccgacatgatcagaaaggctcatgctttatccagaccctggtgg

SEQ ID NO: 133 - DNA, 50 bp, Homo sapiens
acatcgccccttctgcttcagtgtgaaaggccacgtgaagatgctgcggc

SEQ ID NO: 134 - DNA, 50 bp, Homo sapiens
cctgagccagaagtggggtgcttatactcccaaaccttgagtgtccagcc

SEQ ID NO: 135 - DNA, 50 bp, Homo sapiens
aaattgaacacaaatgtggtggagacgggacagggcaggtggaaattcac

SEQ ID NO: 136 - DNA, 50 bp, Homo sapiens
ggcagagaaggaggccaagaagccaaccatcaagaagcccctcaatgcct

SEQ ID NO: 137 - DNA, 50 bp, Homo sapiens
cccaggctggtcttacagcctcaggcaatcctctggtcttgacgtcccaa

SEQ ID NO: 138 - DNA, 50 bp, Homo sapiens
aggccgagtggtttgaggacgatgtcatacagcgcaagagggagctgtgg

SEQ ID NO: 139 - DNA, 50 bp, Homo sapiens
taacttccaggagttcctcattctggtgataaagatgggcgtggcagccc

SEQ ID NO: 140 - DNA, 50 bp, Homo sapiens
cttcctgattgacaacagtgttagacaaggtgcaaagcgaaactggttgc

SEQ ID NO: 141 - DNA, 50 bp, Homo sapiens
agtgctcctgttgcaggactgctgggaaaacaggtggtgtgggacttaag

SEQ ID NO: 142 - DNA, 50 bp, Homo sapiens
cagccagtgccaacttcgctgccaactttggtgccattggtttcttctgg

SEQ ID NO: 143 - DNA, 50 bp, Homo sapiens
gaagttgtcacctccctacagctccccacaggagtttgcccaggatgtgg

SEQ ID NO: 144 - DNA, 50 bp, Homo sapiens
acagctttgctactgcgaaatcttggcttcactgccatccccctccatgg

SEQ ID NO: 145 - DNA, 50 bp, Homo sapiens
ccccacccccgcgttccgaccgctgaagctccaaattcaggccttaaata

SEQ ID NO: 146 - RNA, 22 nt, Homo sapiens
agagguaguagguugcauaguu

SEQ ID NO: 147 - RNA, 22 nt, Homo sapiens
caaagaauucuccuuuugggcu

SEQ ID NO: 148 - DNA, 50 bp, Homo sapiens
catttctgtaaggcaatcttggcacacgtggggcttaccagtggcccagg

SEQ ID NO: 149 - DNA, 50 bp, Homo sapiens
cctgtgccttgccagtgggattccttgtgtgtctcatgtctgggtccatg

SEQ ID NO: 150 - DNA, 50 bp, Homo sapiens
gaagcatacccagggaagaagctgttgccggatgacccctatgagaaagc

SEQ ID NO: 151 - RNA, 21 nt, Homo sapiens
uaaagugcugacagugcagau

SEQ ID NO: 152 - DNA, 50 bp, Homo sapiens
cgacactgactactgaccgtgcgggtgctctcaccctcccttctctccct

SEQ ID NO: 153 - DNA, 50 bp, Homo sapiens
tctgtgccctttatccgcacttcccagctcacagcactgacaaccggtga

SEQ ID NO: 154 - DNA, 50 bp, Homo sapiens
gcacccagcggaatgtgcttagtatttggtcaccagccgtcatcctgggc

SEQ ID NO: 155 - DNA, 50 bp, Homo sapiens; identical to SEQ ID NO: 24

SEQ ID NO: 156 - DNA, 50 bp, Homo sapiens
gcatctccagtgcctggacagattccaattcacagagcacaggtgccacc

SEQ ID NO: 157 - DNA, 50 bp, Homo sapiens; identical to SEQ ID NO: 27

SEQ ID NO: 158 - RNA, 23 nt, Homo sapiens
uaaggugcaucuagugcagauag

SEQ ID NO: 159 - DNA, 50 bp, Homo sapiens; identical to SEQ ID NO: 7

SEQ ID NO: 160 - DNA, 50 bp, Homo sapiens
gctcaaggctggcaaaatcccaaaaccagggccaaggagtggacgcttct

SEQ ID NO: 161 - DNA, 50 bp, Homo sapiens
ggctggagctgggagaggtgctgagctaacagtgccaacaagtgctcctt

SEQ ID NO: 162 - DNA, 50 bp, Homo sapiens; identical to SEQ ID NO: 17

SEQ ID NO: 163 - DNA, 50 bp, Homo sapiens
ggcagtacagggcaccatcactgaccttcccgaccacttactctcctatg

SEQ ID NO: 164 - DNA, 50 bp, Homo sapiens
ggatggcctggaacccatgtcagtctctcaccacctccagcttcgatgat

SEQ ID NO: 165 - DNA, 50 bp, Homo sapiens
ttgcttgtgtgcatgtgttgggtgcatgcttccgggtctcagctgcccca

SEQ ID NO: 166 - RNA, 23 nt, Homo sapiens
ugagcgccucgacgacagagccg

SEQ ID NO: 167 - DNA, 50 bp, Homo sapiens
gggccttatttccactttgtaattccagcgagtcgacttcccatcctgag

SEQ ID NO: 168 - DNA, 50 bp, Homo sapiens
acttaaaaaatacttcgtttatcacatctcaggaactaaactgggttaag

SEQ ID NO: 169 - DNA, 50 bp, Homo sapiens
tgcaagggacagggggcctgactacccagtctttgacttgtatcctctcc

SEQ ID NO: 170 - DNA, 50 bp, Homo sapiens
tcacttgggagggacgcatagaaggagctctaggaacacagtgccagtgc

SEQ ID NO: 171 - DNA, 50 bp, Homo sapiens
gtgcctcaggttaatggtgaaaatacagagagacatgctcagccaccacc

SEQ ID NO: 172 - DNA, 50 bp, Homo sapiens
gacctgactccactcttaaacctgggtcttctccttggcggtgctgtcag

SEQ ID NO: 173 - DNA, 50 bp, Homo sapiens
tctgatcttcctgcggctgaaccgcccggctgagccgacattgccggcgt

SEQ ID NO: 174 - DNA, 50 bp, Homo sapiens
tgaggctctggtgctcagggggatggcttgggccttttctctcaaccttg

SEQ ID NO: 175 - DNA, 50 bp, Homo sapiens
atccagagcatggagcccgaccccagccagcgccttccactccatcattt

SEQ ID NO: 176 - DNA, 50 bp, Homo sapiens
gagggactgtcgctgtgatcagagtgggttaagctgaccaggaacaccca

SEQ ID NO: 177 - DNA, 50 bp, Homo sapiens
ccgagggacggggtcgtttttctctgcgttcagtggatttccgtcttttg

SEQ ID NO: 178 - DNA, 50 bp, Homo sapiens
aggatggcctggaacccatgtcagtctctcaccacctccagcttcgatga

SEQ ID NO: 179 - RNA, 22 nt, Homo sapiens
ugagguaguagguuguauaguu

SEQ ID NO: 180 - DNA, 50 bp, Homo sapiens
gctaaggctggtgtccctttaccaccaaacctaaagcctgcacctccacc

SEQ ID NO: 181 - DNA, 50 bp, Homo sapiens
ctctcctgctgggacaccgcttgggctttggtattgactgagtggctgac

SEQ ID NO: 182 - DNA, 50 bp, Homo sapiens
gtgctctgtaccagtgctcatcatcccttcttcataccaacggtccctag

SEQ ID NO: 183 - DNA, 50 bp, Homo sapiens
cttggcccgagcccctccgtgaggaacacaatctcaatcgttgctgaatc

SEQ ID NO: 184 - DNA, 50 bp, Homo sapiens
cctgctccactggcccaaatcagtaccccaatgttcttgccttctgccca

SEQ ID NO: 185 - DNA, 50 bp, Homo sapiens
taaggccctgcactgaaaatgcaagctcaggcgccggtggtcgttgtgac

SEQ ID NO: 186 - DNA, 50 bp, Homo sapiens
ccagtgtcactatgatgtcagtgaggtctggggatgaggacagtgtgtcc

SEQ ID NO: 187 - DNA, 50 bp, Homo sapiens
cactgatttgacatagtctggctgtacccaggaatggagcctgcacggtg

SEQ ID NO: 188 - DNA, 50 bp, Homo sapiens; identical to SEQ ID NO: 85

SEQ ID NO: 189 - DNA, 50 bp, Homo sapiens
atcgagtcctacaatgctaccctctccgtccatcagttggtagagaacac

SEQ ID NO: 190 - DNA, 50 bp, Homo sapiens
gctggaacctgaagtctaaacaccattcctgctctccagcttcctttccc

SEQ ID NO: 191 - DNA, 50 bp, Homo sapiens
ctgcagctgggagcctgctttctgccagtcttgaggttctgaagatcagc

SEQ ID NO: 192 - DNA, 50 bp, Homo sapiens
ctgtacagtcatgtgccacgtaacagcgtctgggtcagtgacggacactt

SEQ ID NO: 193 - DNA, 50 bp, Homo sapiens
tccctggaactcaataactcatttcactggctctttatcgagagtactag

SEQ ID NO: 194 - DNA, 50 bp, Homo sapiens
gtctggagggaaatctggcgaaaccttcgtttgagggactgatgtgagtg

SEQ ID NO: 195 - DNA, 50 bp, Homo sapiens; identical to SEQ ID NO: 41

SEQ ID NO: 196 - DNA, 50 bp, Homo sapiens
cggacgctgttctaaaaaaggtctcctgcagatctgtctgggctgtgatg

SEQ ID NO: 197 - DNA, 50 bp, Homo sapiens
attgcctctgacgtctggtcttttggagtcactctgcatgagctgctgac

SEQ ID NO: 198 - DNA, 50 bp, Homo sapiens
gctgaggggtaagaggttgttgtagttgtcctggtgcctccatcagactc

SEQ ID NO: 199 - DNA, 50 bp, Homo sapiens
ggaagccaggtgcctttaatccactgtaacctcacaactccaagtccaca

SEQ ID NO: 200 - DNA, 50 bp, Homo sapiens
ctaggactgggcccgagggtggtttacctgcaccgttgactcagtatagt

SEQ ID NO: 201 - DNA, 50 bp, Homo sapiens
ttccgtccaacaactctgtagagctctctgcacccttacccctttccacc

SEQ ID NO: 202 - DNA, 50 bp, Homo sapiens
cataccggctggccacgggaagcgatgataactgcgcggcattctttgag

SEQ ID NO: 203 - DNA, 50 bp, Homo sapiens
gctcctgctgcaaccgctgtgaatgctgctgagaacctccctctatgggg

SEQ ID NO: 204 - DNA, 50 bp, Homo sapiens
cccctggagtccgagaaggaaaatggaattctggttcatactgtggtccc

SEQ ID NO: 205 - DNA, 50 bp, Homo sapiens; identical to SEQ ID NO: 93

SEQ ID NO: 206 - DNA, 50 bp, Homo sapiens; identical to SEQ ID NO: 36

SEQ ID NO: 207 - DNA, 50 bp, Homo sapiens
ccggggcttccacctgacttcctggactctgaggtcaacttattcctggt

SEQ ID NO: 208 - DNA, 50 bp, Homo sapiens
agaaagggtttttatggaccaatgccccagttgtcagtcagagccgttgg

SEQ ID NO: 209 - DNA, 50 bp, Homo sapiens
tcctcacaggacagaagcagagtgggtggtggttatgtttgacagaaggc

SEQ ID NO: 210 - RNA, 22 nt, Homo sapiens
uagcuuaucagacugauguuga

SEQ ID NO: 211 - DNA, 50 bp, Homo sapiens; identical to SEQ ID NO: 90

SEQ ID NO: 212 - DNA, 50 bp, Homo sapiens
catatacgtgtgccgggtccaggagggcaacgagtcataccagcagtcct

SEQ ID NO: 213 - DNA, 50 bp, Homo sapiens
gctctgatttccggggcagcctttcagatgcggcagacatacaacacctg

SEQ ID NO: 214 - DNA, 50 bp, Homo sapiens; identical to SEQ ID NO: 104

SEQ ID NO: 215 - DNA, 50 bp, Homo sapiens
cactgccgtcccccaaggtccagaatgtcagctcgcctcacaagtcagaa

SEQ ID NO: 216 - RNA, 22 nt, Homo sapiens
cacuagauugugagcuccugga

SEQ ID NO: 217 - DNA, 50 bp, Homo sapiens
gcatcctcctgtgtatggaagagacaggtgaccgctccaggttgggtgct

SEQ ID NO: 218 - DNA, 50 bp, Homo sapiens
gagccggggcaccttgctgttcgctgctgtgtcgtcttctaatgtgagct

SEQ ID NO: 219 - DNA, 50 bp, Homo sapiens
agataggccagagcgtggacgaggtggagaagctcatcaagcgccacgag

SEQ ID NO: 220 - DNA, 50 bp, Homo sapiens
tcacactggcgctaagccctacaagtgtcaggactgtggaaaagccttcc

SEQ ID NO: 221 - DNA, 50 bp, Homo sapiens
cccctgtgggggccaaagtttttatgtgggcagatgctgtggtcaggaac

SEQ ID NO: 222 - DNA, 50 bp, Homo sapiens
caatggcgtgtacccatgtattgcacaaggagtgtatcaaattctgggcc

SEQ ID NO: 223 - DNA, 50 bp, Homo sapiens
gcaagtacagaaggaatctattctcagcagggcatagggcacgcactggc

SEQ ID NO: 224 - DNA, 50 bp, Homo sapiens
tcgggttcctgcgctgacacctggtctgtgcacctgtgttgctcacagtt

SEQ ID NO: 225 - DNA, 50 bp, Homo sapiens
atgctgtctgtgtggaacaagcgtcgcaatgaggactctctacaggaccc

SEQ ID NO: 226 - DNA, 50 bp, Homo sapiens
gagctctgaagctttgaatcattcagtggtggagatggccttctggtaac

SEQ ID NO: 227 - DNA, 50 bp, Homo sapiens
tggctcccaactcctccctatcctaaaggcccactggcattaaagtgctg

SEQ ID NO: 228 - DNA, 51 bp, Homo sapiens
ctgctccgacagcagccccaggaaatacgggaatggttcagggaccaagtv

SEQ ID NO: 229 - DNA, 50 bp, Homo sapiens
ctcctggaaataaacaagctaattcctctatgccaccagctccagacagt

SEQ ID NO: 230 - DNA, 50 bp, Homo sapiens; identical to SEQ ID NO: 47

SEQ ID NO: 231 - DNA, 50 bp, Homo sapiens
tgggcggggcaggcctcctttgttctccacaatctactgtctccgagtgt

SEQ ID NO: 232 - DNA, 50 bp, Homo sapiens
gagctctaacctctccccgacccctgcagtatctccctttgttcagtctt

SEQ ID NO: 233 - DNA, 50 bp, Homo sapiens
aggctttagccctggacccagcaggtgaggctcggcttggattattctgc

SEQ ID NO: 234 - DNA, 50 bp, Homo sapiens
gatgacacctttgaggccctgtgcatcgagccgttttccagcccgccaga

SEQ ID NO: 235 - DNA, 50 bp, Homo sapiens
tgtactgtaacctcacaactccaagtccacagaatatttcaaactgcagg

SEQ ID NO: 236 - DNA, 50 bp, Homo sapiens
gggagggcaagctggatttacaggtcacggctggactgaatgggcctttt

SEQ ID NO: 237 - DNA, 50 bp, Homo sapiens
tgaggtcagcagtttgtatgagacatagcttcctccattgcccccactcc

SEQ ID NO: 238 - DNA, 50 bp, Homo sapiens
aacatcctcctggcctctgttgggtcagtgttgggggcctgcttggggaa

SEQ ID NO: 239 - DNA, 50 bp, Homo sapiens
ggcaccctgcttcctttgcttgcatcccacagactatttccctcatccta

SEQ ID NO: 240 - DNA, 50 bp, Homo sapiens; identical to SEQ ID NO: 5

SEQ ID NO: 241 - DNA, 50 bp, Homo sapiens
cccctggattgccccagtcctgtgaccatgttgccctgaagaagaccatc

SEQ ID NO: 242 - DNA, 50 bp, Homo sapiens
gctcctgcctctctcccaacatgtttccagcaagtagatgcccctgtgtg

SEQ ID NO: 243 - DNA, 50 bp, Homo sapiens
tggcccaggagactgacccaaagtgaaggacattgccgggagaggcctgc

SEQ ID NO: 244 - DNA, 50 bp, Homo sapiens
cttttgcttcaggctaagagctgcctcgctctttgtccccccattaggat

SEQ ID NO: 245 - DNA, 50 bp, Homo sapiens
aggaggcgaagcccgcagagcaaaggtggaaacacgtgcctacgctgtaa

SEQ ID NO: 246 - RNA, 23 nt, Homo sapiens
agcagcauuguacagggcuauga

SEQ ID NO: 247 - DNA, 50 bp, Homo sapiens; identical to SEQ ID NO: 12

SEQ ID NO: 248 - DNA, 50 bp, Homo sapiens
gcagtggtgtcgttcaccgtgagagtctgcatagaactcagcagtgtgcc

SEQ ID NO: 249 - DNA, 50 bp, Homo sapiens
ccccattcggtgtggtgcagtgtgaaaagtccttgattgttcgggtgtgc

SEQ ID NO: 250 - DNA, 50 bp, Homo sapiens
tgcctctgcccagctccccattcacacacaccggcactttcataccctga

SEQ ID NO: 251 - DNA, 50 bp, Homo sapiens
cggctacagcttcaccaccacggccgagcgggaaatcgtgcgtgacatta

SEQ ID NO: 252 - DNA, 50 bp, Homo sapiens
gccaagcctttccctccctacctgatcactgcttaacggcatgtataatg

SEQ ID NO: 253 - RNA, 22 nt, Homo sapiens
aauugcacgguauccaucugua

SEQ ID NO: 254 - RNA, 21 nt, Homo sapiens
uaccacaggguagaaccacgg

SEQ ID NO: 255 - DNA, 50 bp, Homo sapiens
gtgctgggccggggagtccctgtctctcacagcatctagcagtattatta

SEQ ID NO: 256 - RNA, 22 nt, Homo sapiens
gggagccaggaaguauugaugu

SEQ ID NO: 257 - DNA, 50 bp, Homo sapiens
catgatgggatatccctgcctagatctttcagtgagtctctacctcagct

SEQ ID NO: 258 - DNA, 50 bp, Homo sapiens
gagagaggacagttaggagggacagacagctcttcctttcggagcctggc

SEQ ID NO: 259 - DNA, 50 bp, Homo sapiens
cagtgtctcagtcttttttgccgagaaagcacagtagtctgggactgggc

SEQ ID NO: 260 - DNA, 50 bp, Homo sapiens
cagctcggaggaaggtctcctatacacacaaagcctggcatgcaccttcg

SEQ ID NO: 261 - DNA, 50 bp, Homo sapiens; identical to SEQ ID NO: 110

SEQ ID NO: 262 - DNA, 50 bp, Homo sapiens
tgactggtctcctaaccaaggtgcactgagaagcaatcaacgggtcggtc

SEQ ID NO: 263 - DNA, 50 bp, Homo sapiens
gctggttgaaaagtaccactcccactctgaacatctggccgtccctgcaa

SEQ ID NO: 264 - DNA, 50 bp, Homo sapiens
gccctgaccttcatggtgtctttgaagcccaaccactcggtttccttcgg

SEQ ID NO: 265 - DNA, 50 bp, Homo sapiens; identical to SEQ ID NO: 88

SEQ ID NO: 266 - DNA, 50 bp, Homo sapiens; identical to SEQ ID NO: 125

SEQ ID NO: 267 - DNA, 50 bp, Homo sapiens
caccttggtgcccaccctagctgttgctgtctcctatgccttcatcctct

SEQ ID NO: 268 - DNA, 50 bp, Homo sapiens
ctatactcctttggcccatagctaaggtcatccttccccacaggggtggc

SEQ ID NO: 269 - DNA, 50 bp, Homo sapiens
gagaacagaaatagtggcattgcatgcccagcaagatcgggcccttaccc

SEQ ID NO: 270 - DNA, 50 bp, Homo sapiens; identical to SEQ ID NO: 14

SEQ ID NO: 271 - DNA, 50 bp, Homo sapiens
catctggacccctccccctctatccctaaccctgtctaaactaatggcgc

SEQ ID NO: 272 - DNA, 50 bp, Homo sapiens
tccgcccatgatgctgcccaacggctacgtctacggctacaattctctgc

SEQ ID NO: 273 - DNA, 50 bp, Homo sapiens
ctcttatcccagctgcaaggacagtcgaaggatatgccacctcggttttc

SEQ ID NO: 274 - DNA, 50 bp, Homo sapiens
tggaagggactgcagagagaacagtcgagcagtgaggacactgatgctgc

SEQ ID NO: 275 - DNA, 50 bp, Homo sapiens
gcctgttctctgccattccctagtcatcctgtgcctcaccacagcttgct

SEQ ID NO: 276 - DNA, 50 bp, Homo sapiens
ctgccctgctggctggaaacctggtagtgaaacaataatcccagatccag

SEQ ID NO: 277 - DNA, 50 bp, Homo sapiens
gaccacgtccagtgaagacatttgaggcagcacatctcaggacccaggca

SEQ ID NO: 278 - DNA, 50 bp, Homo sapiens
gcatctccacgctctgaagctgtctttcaaaatgtgtgcactgaccccct

SEQ ID NO: 279 - DNA, 50 bp, Homo sapiens; identical to SEQ ID NO: 31

SEQ ID NO: 280 - DNA, 50 bp, Homo sapiens
gatccatcacaaagcgaagtcatgggagagccacacttgatggtggaata

SEQ ID NO: 281 - DNA, 50 bp, Homo sapiens
acaaagttgttgagccttgcttcttccgttttgccctttgtctcgctcct

SEQ ID NO: 282 - DNA, 50 bp, Homo sapiens
ctgccccagctacagagacggccgaaatgctttcactccttagctttgcc

SEQ ID NO: 283 - DNA, 50 bp, Homo sapiens
ggagaaagagctctctatacactttgttcccgggagctgtcggctggtgg

SEQ ID NO: 284 - DNA, 50 bp, Homo sapiens
agctctgttctgattcaccaggggtccgtcagtagtcattgccacccgcg

SEQ ID NO: 285 - DNA, 50 bp, Homo sapiens
gccagaggagaccagaggcttgggttttgatgaaatccggcaacagcagc

SEQ ID NO: 286 - DNA, 50 bp, Homo sapiens
tggcctttcctacagggagctcagtaacctggacggctctaaggctggaa

SEQ ID NO: 287 - DNA, 50 bp, Homo sapiens
gatgctgggcccctcctcatctccctcaaggatggctacgtacccccaaa

SEQ ID NO: 288 - DNA, 50 bp, Homo sapiens
cctgggcatggaatcctgtggcatccacaaaactaccttcaactccatag

SEQ ID NO: 289 - DNA, 50 bp, Homo sapiens
aagaagccgaaggagccacagccggaacagccacagccaagtacaagtgc

SEQ ID NO: 290 - DNA, 50 bp, Homo sapiens
ccaccatcaccttgaccttcatcgacaagaacggagagactgagctgtgc

SEQ ID NO: 291 - DNA, 50 bp, Homo sapiens
ccataactggagaaagaagctccattgaccgaagccacagggcagcatgg

SEQ ID NO: 292 - DNA, 50 bp, Homo sapiens
acctgaggcccttaacctttctctcagtgctcgccttcccccagaatccc